(19) United States
(12) Patent Application Publication
(10) Pub. No.: US 2016/0115405 A1
(43) Pub. Date: Apr. 28, 2016

(54) Organic Fuel and Waste Reformer

(71) Applicant: Pioneer Astronautics, Lakewood, CO (US)

(72) Inventors: Robert Zubrin, Golden, CO (US); Mark Berggren, Golden, CO (US); Stacy Lorraine Carrera, Golden, CO (US)

(73) Assignee: Pioneer Astronautics, Lakewood, CO (US)

(21) Appl. No.: 14/921,981

(22) Filed: Oct. 23, 2015

Related U.S. Application Data
(60) Provisional application No. 62/068,454, filed on Oct. 24, 2014.

(51) Int. Cl. C10J 3/34; C10J 3/72; C25B 1/04; C07C 1/04; C10K 3/00; C10J 3/06; C10J 3/82

(52) U.S. Cl. CPC C10J 3/34; C10J 3/06; C10J 3/726; C10J 3/82; C07C 1/041; C07C 1/0485; C07C 1/0425; C10K 3/005; C25B 1/04; C10J 2300/0916; C10J 2300/094; C10J 2300/0959; C10J 2300/0976; C10J 2300/0986; C10J 2300/1853; C10J 2300/1659

(57) ABSTRACT

This invention pertains to the non-catalytic oxygenated steam reforming of organic matter to produce a gas mixture rich in hydrogen, carbon monoxide and carbon dioxide. The reforming gas is used for production of methane, methanol, dimethyl ether, oxygen, carbon dioxide, and other compounds via downstream processing catalytic gas-phase processes and electrolysis. The reforming gas may also be combusted directly for electricity generation.

### RELATED APPLICATIONS

This application claims the benefit of U.S. Provisional Application No. 62/068,454 filed Oct. 24, 2014 entitled “Organic Waste Reformer” which is incorporated herein by reference in its entirety.

### GOVERNMENT SUPPORT STATEMENT

This invention was made with Government support under contract NNX11CA76C awarded by NASA. The Government has certain rights in this invention.

### BACKGROUND OF THE INVENTION

If energy is available, one way to transform organic fuel and waste products into useful gases is steam reformation. In one example, steam reformation is used to produce synthesis gas from coal or other carbonaceous sources via reaction (1).

C+H₂O=>CO+H₂ ΔH=+136 kJ at 750° C. (1)

If more steam is available, the CO will be converted to CO₂ via the water gas shift reaction (2).

CO+H₂O=>CO₂+H₂ ΔH=−35 kJ at 750° C. (2)

Taken together, the net result of reactions (1) and (2) is reaction (3)

C+2H₂O=>CO₂+2H₂ ΔH=+101 kJ at 750° C. (3)

Similarly, steam reformation can be used to produce mixtures of CO₂, H₂, and CO from any organic material. An example organic matter composition can be steam reformed via reaction (4). The example reaction is shown without the presence of excess moisture that may be used to boost carbon conversion in organic matter to gases.

C₆H₁₂O₆+4H₂O=>4CO₂+2CO+12H₂ (4)

In short, regardless of the details of its composition, if the material is organic, it can be reformed into a gas mixture predominantly composed of CO₂, CO, and H₂ by reaction with high temperature steam.

As detailed in this application, oxygen can be introduced with steam to provide an exothermic heat of reaction to drive the endothermic steam reforming reactions as an alternative to indirect heating of the organic matter and steam subjected to reforming as described above.

The Organic Waste Reformer is a novel technology to convert organic matter into useful methane plus oxygen constituents when integrated with a methanation reactor including a gas separation system and electrolysis. The terms “methanation” and “Sabatier reaction” are considered to be comparable descriptions of converting carbon monoxide and carbon dioxide into methane for the purposes of this application.

The Organic Waste Reformer is a novel technology to convert organic matter into useful methane plus carbon dioxide constituents when integrated with a methanation reactor system including a gas separation system.

The Organic Waste Reformer is a novel technology to convert organic matter into methanol when integrated with a methanol synthesis reactor.

The Organic Waste Reformer is a novel technology to convert organic matter into dimethyl ether when integrated with a combined methanol-dimethyl ether synthesis reactor system.

The Organic Waste Reformer is a novel technology to convert organic matter into a gaseous fuel for production of electricity when integrated with combustion-turbine, steam-turbine, or internal combustion power generation technology.

The Organic Waste Reformer first converts liquid, solid, or gaseous organic matter into a mixture consisting mostly of hydrogen, carbon monoxide, and carbon dioxide gas via oxygenated steam reforming

Oxygen is consumed during exothermic partial oxidation of organic matter to generate heat that simultaneously supports endothermic steam reforming reactions.

The hydrogen, carbon monoxide, and carbon dioxide reformer gases are then fed to a methanation reactor to produce a mixture of methane and carbon dioxide (see FIG. 1).

With the integration of an electrolysis module, the oxygenated steam reformer and methanation systems convert organic matter into methane and oxygen products (see FIG. 2).

Optionally, the hydrogen, carbon monoxide, and carbon dioxide reformer gases are fed to a combined methanol-dimethyl ether synthesis reactor to produce dimethyl ether and methanol products (see FIG. 7).

Optionally, the hydrogen, carbon monoxide, and carbon dioxide reformer gases are fed to a methanol synthesis reactor to produce methanol product (see FIG. 11).

The Organic Waste Reformer reduces landfill volume, health risks, and environmental runoff and emissions associated with storing, handling, and disposing residential and commercial organic matter such as food waste and packaging, waste paper, fecal matter, urine brine, diapers, and other organic fuel and wastes. Similarly, agricultural and industrial organic wastes, crops, and crop residues can be converted to valuable products.

Products generated by the Organic Waste Reformer system reduce the need to produce and consume new resources such as natural gas for heating or power generation, carbon dioxide for carbonation or enhanced oil recovery, hydrogen for chemical synthesis or energy production, dimethyl ether and methanol for fuel or chemical feedstock.

The Organic Waste Reformer requires minimal feed preparation and results in nearly complete conversion of the organic matter in feeds to valuable products with minimal losses and consumables requirements.

Inorganic byproducts from reforming such as metals and metal oxides can be recovered for further recycling and processing into useful materials.

Effective heat exchange within the Organic Waste Reformer and downstream systems results in very high thermal efficiency.

For an integrated reformer-methanation-electrolysis system (FIG. 2), thermo-chemical production of hydrogen in the reformer reduces electrolysis requirements by up to 40 percent compared to combustion-based organic waste treatment methods aimed toward methane production.

For an integrated reformer-dimethyl ether-methanol synthesis system (FIG. 7), valuable dimethyl ether is produced at high yields relative to step-wise production of methanol followed by dimethyl ether synthesis.

The features listed above also make the Organic Waste Reformer system integrated with methanation and electrolysis applicable to human space exploration missions to manufacture methane and oxygen products. Oxygenated steam reforming of organic wastes in an enclosed spacecraft or habitat life support system substantially reduces requirements for resupply of consumables from Earth, thereby saving significant costs and minimizing mission risk.

### BRIEF DESCRIPTION OF DRAWINGS

FIG. 1. Integrated Oxygenated Steam Reformer—Methanation Flow Diagram.

FIG. 2. Integrated Oxygenated Steam Reformer-Methanation-Electrolysis Flow Diagram.

FIG. 3. Oxygenated Steam Reformer Vessel, Startup Boiler, and Oxygen Flow Controller.

FIG. 4. Schematic of Oxygenated Steam Reformer Vessel.

FIG. 5. Methanation Reactor and Membrane Separation System.

FIG. 6. Methanation Reactor.

FIG. 7. Integrated Oxygenated Steam Reformer—Dimethyl Ether Synthesis Flow Diagram

FIG. 8. Lunar Organic Waste Reformer Process Flow Diagram.

FIG. 9. SolidWorks drawing of Condensing System.

FIG. 10. Integrated Oxygenated Steam Reformer—RWGS—Dimethyl Ether Synthesis Flow Diagram.

FIG. 11. Integrated Oxygenated Steam Reformer—Methanol Synthesis Flow Diagram.

FIG. 12. Integrated Oxygenated Steam Reformer—RWGS—Methanol Synthesis Flow Diagram.

FIG. 13. Reactor Thermal Control System.

FIG. 14. Steam reformer temperature profile and dry gas analysis.

FIG. 15. Temperatures within steam generator.

FIG. 16. Temperatures around heat exchanger.

FIG. 17. Pressures around the heat exchanger.

FIG. 18. Absolute pressure profile of steam reformer.

FIG. 19. System exhaust flow rate as measured by the dry test meter.

FIG. 20. Dry gas analysis with gas chromatograph.

FIG. 21. Steam to oxygen ratio and the resulting carbon monoxide to carbon dioxide volume percent ratio from steam reduction test.

FIG. 22. Effect of H₂:C ratio on adiabatic Sabatier reaction temperature.

FIG. 23. Effect of excess Sabatier reactor feed hydrogen on exhaust gas composition.

FIG. 24. Sabatier-membrane-recycle system material balance (50-psig retentate pressure).

FIG. 25. Temperature profile of Steam Reformer.

FIG. 26. Temperature profile of steam generator.

FIG. 27. Temperature profile of heat exchanger with steam reformer and water input to steam generator.

FIG. 28. Oxygen control pressure profile.

FIG. 29. Pressure profile of steam reformer and components.

FIG. 30. Sabatier Temperature profile.

FIG. 31. Membrane Separator flow rates and retentate back pressure.

FIG. 32. Material balance during integrated LOWR shakedown experiment.

FIG. 33. Retentate (Methane Product) gas sensor data during 02-15-2013 operation.

FIG. 34. LOWR mass balance (40 psig retentate backpressure).

FIG. 35. Retentate (Methane Product) gas sensor data during 02-28-2013 operation.

FIG. 36. Pressure profile during integrated operation.

FIG. 37. Flow profile during 02-28-2013 integrated operation.

FIG. 38. LOWR mass balance 1 (˜40 psig retentate backpressure).

FIG. 39. LOWR mass balance 2 (˜40 psig retentate backpressure).

FIG. 40. LOWR mass balance 1 (˜40 psig retentate backpressure).

FIG. 41. LOWR mass balance 2 (˜40 psig retentate backpressure).

FIG. 42. LOWR mass balance 3 (˜40 psig retentate backpressure).

FIG. 43. LOWR mass balance 1 (˜30 psig retentate backpressure) (NASA High Fidelity Waste Simulant).

FIG. 44. LOWR mass balance 2 (˜30 psig retentate backpressure) (NASA High Fidelity Waste Simulant).

FIG. 45. Steam reformer temperature profile during integrated operation.

FIG. 46. Sabatier temperature profile during integrated operation.

FIG. 47. Flow rates for LOWR profile during integrated operation.

FIG. 48. Pressure profile for LOWR profile during integrated operation.

FIG. 49. Retentate (Methane Product) gas sensor data during integrated operation.

FIG. 50. LOWR mass balance 1 (˜36 psig retentate backpressure) (NASA High Fidelity Waste Simulant).

FIG. 51. Steam reformer temperature profile during integrated operation.

FIG. 52. Sabatier temperature profile during integrated operation.

FIG. 53. Pressure profiles for LOWR during integrated operation.

FIG. 54. Flow rates for LOWR during integrated operation.

FIG. 55. Retentate (Methane Product) gas sensor data during operation.

FIG. 56. Final LOWR mass balance (˜30 psig retentate backpressure) (NASA High Fidelity Waste Simulant).

FIG. 57. LOWR model mass balance (NASA High-Fidelity Fuel and Waste Simulant; Crew of four).

FIG. 58. Effect of olivine on tar formation (6-1-2012 and 6-5-2012 no olivine; 6-20-2-2012 with olivine).

FIG. 59. Effect of distillation of reformer condensate contaminant concentrations.

FIG. 60. High-temperature oxidation of reformer condensate.

### DETAILED DESCRIPTION OF THE INVENTION

As described in this patent application, oxygen can be introduced with steam to partially combust the organic matter with the attendant release of energy. In this manner, complex organic materials are decomposed and then readily react with steam to form the desired reformation product gases. Example partial oxidation reactions are shown in (5) and (6) below.

C+½O₂=>CO ΔH=−112 kJ at 750° C. (5)

CO+½O₂=>CO₂ ΔH=−283 kJ at 750° C. (6)

The exothermic partial oxidation reactions shown in (5) and (6) above supply thermal energy to support non-catalytic endothermic steam reforming. The energy released from partial combustion is immediately consumed by the reforming reactions described above in a concentrated zone of the reforming reactor where the oxygenated steam input contacts the organic matter. The oxygen flow rate controls the rate of the consumption of organic matter. The steam rate is set to provide a stoichiometric excess to push the reforming reactions to completion and to provide reactor temperature control. A molar ratio of oxygen-to-carbon is about 0.5. A typical molar ratio of water-to-carbon is about two. The oxygen and steam molar ratios may vary considerably depending on the composition of the organic matter fed to the reformer and the moisture content contained in the organic feed. Higher organic matter moisture content will require greater oxygen input in order to provide energy to heat and vaporize moisture contained in the organic feed.

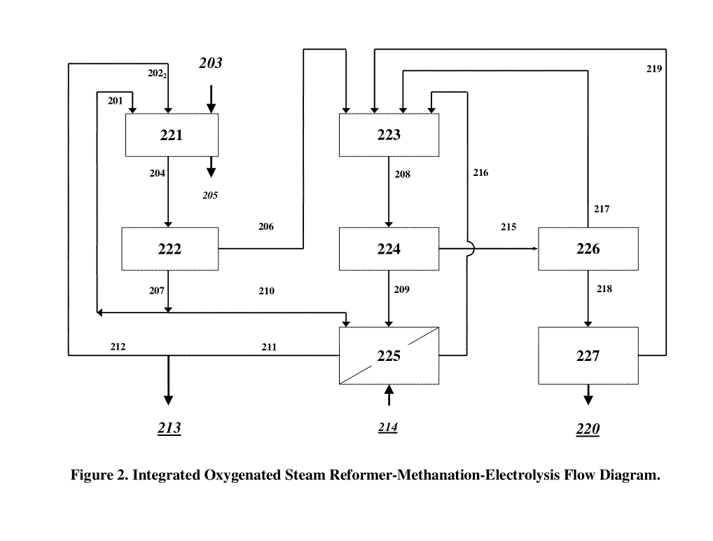

The exhaust gas from oxygenated steam reforming generally contains excess water in addition to CO, CO₂, H₂ and smaller concentrations of other compounds such as methane and other hydrocarbons. In a case where supplemental hydrogen is used to allow full conversion of the carbon oxides to methane (see FIG. 2), the excess water is normally condensed to produce a dry gas that is fed to methanation. Methanation proceeds over a ruthenium, nickel, or other suitable catalyst at temperatures between about 250 and 600° C. according to the following reactions.

CO+3H₂=>CH₄+H₂O ΔH=−219 kJ at 400° C. (7)

CO₂+4H₂=>CH₄+2H₂O ΔH=−181 kJ at 400° C. (8)

Hydrogen present in the reformer exhaust gas is supplemented with additional hydrogen from electrolysis or other sources to satisfy the requirements for reactions in (7) and (8) above. Equilibrium constants for the methanation reactions are high at stoichiometric hydrogen amounts, leading to high per-pass conversion of carbon oxides to methane. However, in a single-pass methanation reactor, some unreacted carbon oxides (predominately CO₂) and unreacted hydrogen will be present with the methane and water produced by methanation reactions.

An electrolysis system is integrated with the oxygenated steam reformer system, water is split into hydrogen and oxygen gas as illustrated in (9).

H₂O=>H₂+½O₂ ΔH=286 kJ at 25° C. (9)

The electrolysis rate for an integrated reformer-methanation-electrolysis system is generally set to provide only the supplemental hydrogen required for methanation of CO and CO₂ and no excess. A portion of the oxygen co-produced with hydrogen by electrolysis is used for partial oxidation in the reformer while any remainder is collected as a byproduct.

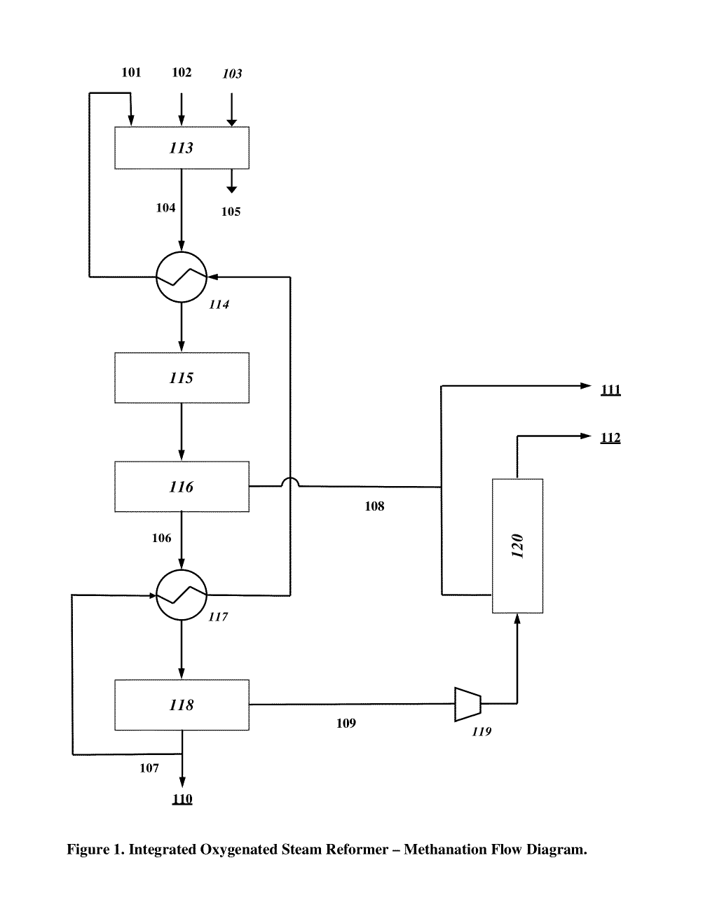

In a case where electrolysis is not integrated with an oxygenated steam reformer and methanation system (see FIG. 1), there will typically be insufficient hydrogen to fully convert carbon oxides to methane. In one example, an agricultural residue or waste feed containing about ten percent moisture and 47 percent carbon, five percent hydrogen, and 38 percent oxygen (all by weight) is subjected to oxygenated steam reformation and subsequent methanation. The approximate composition for such a feed would be C₃₄H₄₆O₂₀. During reforming, ratios of approximately 2:1 H₂O:C and 0.5:1 O₂:C (by mole) are used, and the resulting gas mixture contains an approximate 2:1 ratio of CO₂:CO (by mole). Moisture and oxygen present in the organic matter reduces the amounts of oxygen and steam. An example reforming reaction for this case is shown below. Values were rounded for clarity.

68 H₂O+C₃₄H₄₆O₂₀+7O₂=>46 H₂+23CO₂+12CO+45H₂O (10)

Note that excess water is used to drive the conversion of the organic matter to gaseous products and to control reaction temperature. This water present in the reformer gas exhaust can optionally be removed prior to methanation or may be left in the methanation feed gas to reduce the possibility of solid carbon formation. During methanation, carbon monoxide is first converted to methane using available hydrogen in the reforming gas and the balance of available hydrogen is then consumed to convert carbon dioxide to methane. Thermodynamic equilibrium indicates small amounts of unreacted hydrogen and carbon dioxide are present after a single pass through a methanation reactor under these conditions. However, the use of a gas separation membrane and recycle of hydrogen- and carbon dioxide-rich gas to the methanation reactor (such as depicted in FIG. 1) would result in virtually complete conversion of hydrogen. An example methanation reaction for the reforming gas product (after removing moisture) is shown below. Values were rounded for clarity.

46H₂+23CO₂+12 CO=>15CH₄+20CO₂+18H₂O (11)

Results of the above example show that after removing water from the methanation product gas, methane-rich and carbon dioxide-rich product gases in the proportions shown above would be obtained when following the general flow sheet shown in FIG. 1.

In an implementation where supplemental hydrogen is added from sources such as electrolysis (such as that shown in FIG. 2), the carbon oxides contained in the reforming product gases would be converted nearly entirely to methane. Thermodynamic equilibrium indicates that small amounts of unreacted hydrogen and carbon dioxide would be present after a single pass through a methanation reactor when using the stoichiometric amount of hydrogen required to convert carbon monoxide and carbon dioxide to methane. However, the use of a gas separation membrane and recycle of hydrogen- and carbon dioxide-rich gas to the methanation reactor (such as depicted in FIG. 2) would result in a local excess of hydrogen in the methanation reactor and virtually complete conversion of carbon oxides to methane. An example methanation reaction for the reforming gas product from equation (10) above (after removing moisture) is shown below based on the stoichiometric hydrogen addition required for methanation. Values were rounded for clarity.

128 H₂+23CO₂+12CO=>35CH₄+58H₂O (12)

For the example flow sheet shown in FIG. 2, the water produced by the methanation reaction would be directed to electrolysis, which would produce the necessary hydrogen for complete conversion of carbon oxides to methane while producing oxygen for reforming plus some excess oxygen as product. In some cases, supplemental water is fed to electrolysis to provide the required hydrogen.

The oxygenated steam reforming exhaust gas produced as described above may also be fed to a reactor system for synthesis of methanol (over copper-zinc oxide or other suitable catalyst) and simultaneous conversion of methanol to dimethyl ether (over gamma alumina or other suitable catalyst) at temperatures in the range of 190 to 250° C. The combined methanol-dimethyl ether synthesis approach provides for substantially greater single-pass yield than can be obtained from sequential synthesis of methanol followed by dimethyl ether synthesis from methanol. The hydrogen and carbon monoxide present in dry reformer exhaust gas react to form methanol according to the following exothermic reaction.

CO+2H₂=>CH₃OH ΔH=−98 kJ at 230° C. (13)

The resulting methanol is then immediately reacted to dimethyl ether according to the following exothermic reaction.

2CH₃OH=>C₂H₆O+CO₂ ΔH=−22 kJ at 230° C. (14)

The overall result by adding twice the amounts in equation (13) to equation (14) is as follows.

2CO+4H₂=>C₂H₆O+CO₂ ΔH=−218 kJ at 230° C. (15)

Reactions (13) through (15) are all favored at lower temperature and higher pressure. The lower temperature limit for the required initial formation of methanol is about 200° C. based on industrial practice. Somewhat higher temperatures reduce the yield but improve reaction kinetics. In an example case, the equilibrium constant of equation (13) is low at a practical operating temperature of 230° C., which limits the per-pass yield of methanol to about 35 percent at a pressure of 20 bar. If the methanol produced by equation (13) were separated from the unreacted gases and then fed to a separate dimethyl ether synthesis reactor, about 90 percent of the methanol would be converted to dimethyl ether at a temperature of 230° C. and a pressure of 20 bar. Therefore, the yield of dimethyl ether from a sequential synthesis would be on the order of 32 percent from the feed gas of hydrogen and carbon monoxide.

Alternatively, if the methanol synthesis and dimethyl ether synthesis were carried out in a single reactor with mixed catalyst according to reaction (15) above, the dimethyl ether yield at the same 230° C. and 20 bar would be about 70 percent, or nearly double that of sequential synthesis. The combined reaction takes advantage of the fact that methanol produced via reaction (13) is readily consumed by reaction (14), which enables continued methanol synthesis and subsequent dimethyl ether synthesis in a single vessel. In addition, the process is simplified by combining the reactions into a single vessel. The result of such combined synthesis reaction is that a large majority of the carbon monoxide produced by oxygenated steam reforming can be converted to a valuable dimethyl ether product. A later example illustrates the benefits of such combined synthesis even in the presence of non-reactive diluent gases.

The required H₂:CO ratio for dimethyl ether production is 2:1 as shown in equation (15) above. Depending on the composition of organic matter fed to the reformer, the H₂:CO of the dry reformer product gas may be significantly greater than two. Because hydrogen does not react to any great extent with carbon dioxide during the methanol-dimethyl ether synthesis step, significant unreacted hydrogen will remain after the methanol-dimethyl ether synthesis step. Additional yield of dimethyl ether can be obtained by performing a reverse water gas shift (RWGS) reaction on the dry reformer product gas prior to feeding to the methanol-dimethyl ether reactor. The RWGS reaction is shown below.

CO₂+H₂=>CO+H₂O ΔH=+38 kJ at 400° C. (16)

By conducting a reverse water gas shift reaction to obtain an approximate 2:1 H₂:CO ratio, additional carbon monoxide becomes available to react with hydrogen to form more of the desired dimethyl ether product.

An important aspect of the present invention is a process for converting organic fuel and waste or other organic solids, liquids, and gases into useful products.

In one embodiment organic household wastes are collected and transported to a larger-scale centralized steam reforming facility, to a smaller-scale decentralized steam reforming facility or to an on-site steam reforming facility.

In one embodiment organic agriculture, forestry, or fishing wastes are collected and transported to a larger-scale centralized steam reforming facility, to a smaller-scale decentralized steam reforming facility or to an on-site steam reforming facility.

In one embodiment organic construction wastes are collected and transported to a larger-scale centralized steam reforming facility, to a smaller-scale decentralized steam reforming facility or to an on-site steam reforming facility.

In one embodiment organic manufacturing or industrial wastes are collected and transported to a larger-scale centralized steam reforming facility, to a smaller-scale decentralized steam reforming facility or to an on-site steam reforming facility.

In one embodiment organic municipal or sanitary wastes are collected and transported to a larger-scale centralized steam reforming facility, to a smaller-scale decentralized steam reforming facility or to an on-site steam reforming facility.

In one embodiment organic medical wastes are collected and transported to a larger-scale centralized steam reforming facility, to a smaller-scale decentralized steam reforming facility or to an on-site steam reforming facility.

In one embodiment organic chemicals, fuels, or related wastes from chemicals or fuels including those derived from petroleum, lignite, coal, shale, natural gas, and others are collected and transported to a larger-scale centralized steam reforming facility, to a smaller-scale decentralized steam reforming facility or to an on-site steam reforming facility.

In one embodiment mixed organic fuels or wastes from one or more categories listed above are collected and transported to a larger-scale centralized steam reforming facility, to a smaller-scale decentralized steam reforming facility, or to an on-site steam reforming facility.

In one embodiment there is provided an oxygenated steam reforming apparatus for converting organic fuels or wastes into a gaseous product consisting mostly of hydrogen, carbon dioxide, carbon monoxide, and water vapor along with smaller amounts of methane and other hydrocarbons.

In one embodiment, water for steam reforming is added to the oxygenated steam reforming apparatus from sources including the organic waste material itself or an external water supply that is pumped or otherwise injected directly into the stream reformer or alternatively into a boiler or heat exchanger prior to injection into the steam reformer.

In one embodiment, oxygen for reforming is added to the oxygenated steam reforming apparatus from sources including cryogenically produced pure oxygen, concentrated or partially concentrated oxygen produced by pressure-swing, vacuum-swing, temperature-swing, or other absorption methods, membrane separation, or other oxygen concentration or nitrogen removal devices.

In one embodiment, oxygen for reforming is added to the oxygenated steam reforming apparatus in the form of air.

In one embodiment the water used for reforming is a component of the organic fuel and waste feed.

In one embodiment the water for reforming is supplied separately and is added to the organic fuel and waste feed or is injected separately or together with the oxygen source.

In one embodiment the water for reforming is preheated in a boiler or steam generator.

In one embodiment the water for reforming is recovered from the reformer exhaust and is recycled to the oxygenated steam reformer.

In one embodiment the water for reforming is preheated using waste heat from the oxygenated steam reformer or from associated process hardware such as the methanation system or other thermal processing systems.

In one embodiment, oxygen is preheated using an electric, gas, or other fuel-fired heater.

In one embodiment oxygen is preheated using waste heat from the oxygenated steam reformer or from associated process hardware such as the methanation system or other thermal processing systems.

In one embodiment oxygen is mixed with steam prior to injection into the reformer.

In one embodiment oxygen and steam or water are separately injected into the reformer.

In one embodiment the oxygen and moisture feed rates and concentrations are adjusted to achieve a minimum temperature of 500 degrees centigrade in the reformer reaction zone.

In one embodiment the oxygen and moisture feed rates and concentrations are adjusted to achieve a maximum temperature of 1500 degrees centigrade in the reformer reaction zone.

In one embodiment the oxygenated steam reforming reactor diameter is selected to provide an adequate volume for partial oxidation and steam reforming reactions to take place.

In one embodiment the oxygenated steam reformer is operated in the absence of a reforming catalyst.

In one embodiment the organic matter is first dried or partially dried to adjust moisture to a preferred amount.

In one embodiment the organic matter is fed as-is to the reformer.

In one embodiment the organic matter feed is first shredded, chopped, ground, or otherwise reduced in size before feeding to the reformer.

In one embodiment the organic matter feed is compacted or pelletized before feeding to the reformer.

In one embodiment chemical compounds or absorbents are added to the organic matter feed to trap contaminants such as sulfur, chlorine, fluorine, and other gaseous compounds.

In one embodiment gas contaminant traps are located in the hot, moist reformer exhaust piping.

In one embodiment gas contaminant traps are located in the dried reformer exhaust piping.

In one embodiment the reformer is equipped with a feed magazine which is of a size required to provide continuous feed for a designated operating time.

In one embodiment the reformer is equipped with a continuous or semi-continuous feed system such as a lock-hopper, auger, or other feeding device to allow for extended operating time.

In one embodiment the reformer is operated at pressures near ambient atmospheric pressure.

In one embodiment the reformer is operated at elevated pressures up to 200 bar.

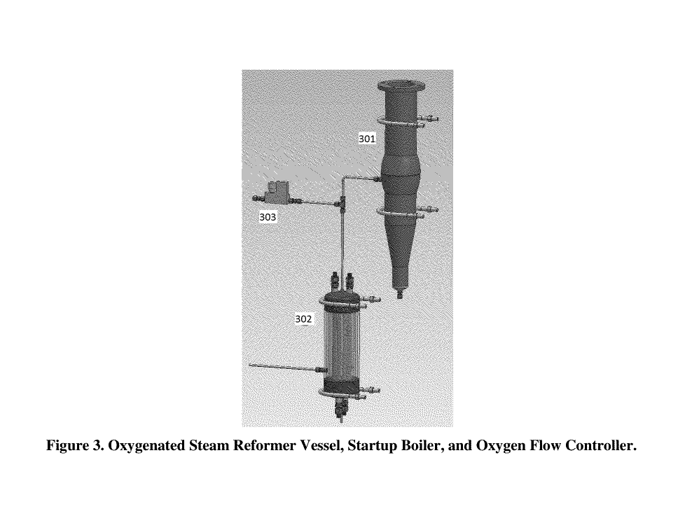

In one embodiment the oxygen and steam are injected into a concentric jacket surrounding all or a portion of the reformer reactor length for purposes of cooling the reformer reactor shell and/or for preheating the oxygen and/or steam (see item 1 in FIG. 3).

In one embodiment single or multiple ports allow the oxygenated steam to enter the reformer reaction zone at one or more positions along the reformer length.

In one embodiment charcoal, carbon, or unprocessed organic matter residue is loaded into the reformer at a location down stream of the reaction zone to provide a physical support for the organic matter being processed and to provide a back up source of fuel in the event of feed depletion or other process upset.

In one embodiment the oxygen, steam, and organic matter are fed and metered manually.

In one embodiment the oxygen, steam and organic matter are fed and metered through valves and feeding devices via an automated control system.

In one embodiment the water feed rate to the reformer is adjusted to provide a sufficient amount for steam reforming.

In one embodiment the water feed rate to the reformer is adjusted to provide an excess amount for steam reforming in order to boost conversion of organic matter to product gases.

In one embodiment the water feed rate to the reformer is adjusted to provide control of reaction temperatures.

In one embodiment the oxygen feed rate to the reformer is adjusted to provide sufficient heat from partial combustion to carry out steam reforming reactions.

In one embodiment the oxygen feed rate to the reformer is adjusted to control the rate of reforming

In one embodiment the oxygenated steam reforming vessel is periodically stopped to remove accumulated inorganic matter and/or unreacted organic matter.

In one embodiment the oxygenated steam reforming vessel is inverted to remove accumulated inorganic matter and/or unreacted organic matter through the feed flange or seal system when the system is not in operation.

In one embodiment accumulated inorganic matter and/or unreacted organic matter is removed via vacuum or pneumatic gas flow through the feed port when the system is not in operation.

In one embodiment accumulated inorganic matter and/or unreacted organic matter is continuously or periodically removed through a port via an auger, lock hopper, or other material flow device.

In one embodiment a boiler or steam generator is used to vaporize water during start up of the reformer (see item 2 in FIG. 3).

In one embodiment a boiler or steam generator is used during operation to further increase the temperature of water vaporized via heat exchange from the reformer system, methanation system, or other reaction systems.

In one embodiment the hot steam reforming exhaust gases are cooled or partially cooled via heat exchange against water or steam being fed to the reformer.

In one embodiment the hot or partially cooled steam reforming exhaust gases are cooled via radiative heat exchange using air or other cool gases.

In one embodiment the hot or partially cooled steam reforming exhaust gases are cooled via a chiller system.

In one embodiment the moisture contained in the reforming exhaust gases is partially or entirely condensed and removed from the reforming gases prior to subsequent methanation or other reactions.

In one embodiment a part or all of the moisture contained in the reforming gases is left in the hot or partially cooled reforming gases that are fed to down-stream reaction system.

In one embodiment the reformer gases containing hydrogen, carbon monoxide, carbon dioxide, and other minor constituents are fed to a methanation reactor along with moisture to minimize carbon formation.

In one embodiment the reformer gases are fed to a methanation reactor for partial or complete conversion of carbon oxides to methane.

In one embodiment the methanation reactor contains ruthenium, nickel, or other suitable catalyst.

In one embodiment the methanation reactor is operated at near ambient pressure.

In one embodiment the methanation reactor is operated at pressures up to 200 bar.

In one embodiment the methanation reactor is operated at a temperature above 250 degrees centigrade.

In one embodiment the methanation reactor is operated at a temperature below 600 degrees centigrade.

In one embodiment the reformer gases contain insufficient hydrogen to convert all carbon oxides to methane, resulting in a methanation product gas rich in methane and carbon dioxide along with small amounts of hydrogen.

In one embodiment the reformer gases contain insufficient hydrogen to convert all carbon oxides to methane, and supplemental hydrogen from electrolysis or other sources is added to the methanation gas feed to provide for conversion of nearly all or all of the carbon oxides to methane.

In one embodiment the reformer gases contain insufficient hydrogen to convert all carbon oxides to methane, and supplemental hydrogen from electrolysis or other sources is added to the methanation gas feed to provide for conversion of nearly all or all of the carbon oxides to methane.

In one embodiment the supplemental hydrogen added to reformer exhaust gases is in an amount equal to the stoichiometric requirement to convert carbon oxides to methane with the result that some unreacted carbon oxides and hydrogen are present in the methanation reactor exhaust due to chemical equilibrium limitations.

In one embodiment the supplemental hydrogen added to reformer exhaust gases is in an amount greater than the stoichiometric requirement to convert carbon oxides to methane with the result that nearly all carbon oxides are converted to methane and the excess hydrogen is present in the exhaust gas.

In one embodiment the hot methanation reactor exhaust gases are cooled or partially cooled via heat exchange against water or steam being fed to the reformer.

In one embodiment the hot or partially cooled methanation reactor exhaust gases are cooled via radiative heat exchange using air or other cool gases.

In one embodiment the hot or partially cooled methanation exhaust gases are cooled via a chiller system.

In one embodiment the moisture contained in the methanation reactor exhaust gases is partially or entirely condensed and removed.

In one embodiment the dry product gas resulting from methanation is used directly as fuel or for other uses.

In one embodiment the dry product gas resulting from methanation is stored for later use.

In one embodiment when no supplemental hydrogen or sub-stoichiometric supplemental hydrogen is added to the methanation reactor feed, the resulting methanation exhaust gas is passed through a gas separation membrane to create hydrogen and carbon dioxide-rich and methane-rich product gas streams.

In one embodiment when supplemental hydrogen is added to the methanation reactor feed, the resulting methanation exhaust gas is passed through a gas separation membrane to create hydrogen-rich and methane-rich product gas streams.

In one embodiment when no supplemental hydrogen or sub-stoichiometric supplemental hydrogen is added to the methanation reactor feed, the resulting methanation exhaust gas is passed through a gas separation membrane to create hydrogen and carbon dioxide-rich and methane-rich product gas streams and the hydrogen and carbon dioxide-rich gas stream is recycled to the methanation reactor feed to promote greater conversion of hydrogen and carbon oxides to methane.

In one embodiment when supplemental hydrogen is added to the methanation reactor feed, the resulting methanation exhaust gas is passed through a gas separation membrane to create hydrogen-rich and methane-rich product gas streams and the hydrogen-rich gas stream is recycled to the methanation reactor feed to maintain a driving force for nearly complete conversion of carbon oxides to methane.

In one embodiment the dry methanation exhaust gas is passed through a gas separation membrane to create hydrogen-rich and methane-rich product gas streams and the hydrogen-rich gas stream is recycled to the methanation reactor feed to provide a diluent gas for control of temperatures resulting from the exothermal methanation reactions.

In one embodiment a part of or all of the water present in the steam reformer exhaust and/or the methanation reactor exhaust is condensed and fed to an electrolysis system for production of hydrogen and oxygen gas.

In one embodiment a part or all of the oxygen produced by electrolysis is fed to the steam reformation reactor.

In one embodiment a part or all of the oxygen produced by electrolysis is compressed or liquefied.

In one embodiment a part or all of the oxygen produced by electrolysis is collected for other use or sale.

In one embodiment a part or all of the hydrogen produced by electrolysis is fed to the methanation reactor.

In one embodiment a part or all of the hydrogen produced by electrolysis is compressed or liquefied.

In one embodiment a part or all of the hydrogen produced by electrolysis is collected for other use or sale.

In one embodiment the composition of the product gas from methanation is analyzed in near real time via infrared or other analysis methods.

In one embodiment the composition of the product gas from methanation is used for feedback control of the electrolysis rate for supply of the target amount of hydrogen to the methanation reactor.

In one embodiment the reformer gases are fed to a catalytic reactor for simultaneous partial or complete conversion of carbon oxides to dimethyl ether and methanol.

In one embodiment the simultaneous dimethyl ether and methanol synthesis reactor contains a mixture of copper-zinc oxide plus gamma alumina or other suitable catalysts.

In one embodiment the dimethyl ether-methanol synthesis reactor is operated at near ambient pressure.

In one embodiment the dimethyl ether-methanol synthesis reactor is operated at pressures up to 100 bar.

In one embodiment the dimethyl ether-methanol synthesis reactor is operated at a temperature above 190 degrees centigrade.

In one embodiment the dimethyl ether-methanol synthesis reactor is operated at a temperature below 400 degrees centigrade.

In one embodiment the hot dimethyl ether-methanol synthesis reactor exhaust gases are cooled or partially cooled via heat exchange against water or steam being fed to the reformer.

In one embodiment the hot or partially cooled dimethyl ether-methanol synthesis reactor exhaust gases are cooled via radiative heat exchange using air or other cool gases.

In one embodiment the hot or partially cooled dimethyl ether-methanol synthesis exhaust gases are cooled via a chiller system.

In one embodiment the moisture contained in the dimethyl ether-methanol synthesis reactor exhaust gases is partially or entirely condensed and removed.

In one embodiment the dimethyl ether and methanol are partially or entirely condensed and removed.

In one embodiment the dimethyl ether and methanol are distilled from the water in the synthesis reactor exhaust.

In one embodiment the dry product gas resulting from dimethyl ether-methanol synthesis is used directly as fuel or for other uses.

In one embodiment the dry product gas resulting from dimethyl ether-methanol synthesis is stored for later use.

In one embodiment the reformer gases are fed to a catalytic reactor for simultaneous partial or complete conversion of carbon oxides to methanol.

In one embodiment the methanol synthesis reactor contains copper-zinc oxide or other suitable catalysts.

In one embodiment the methanol synthesis reactor is operated at near ambient pressure.

In one embodiment the methanol synthesis reactor is operated at pressures up to 100 bar.

In one embodiment the methanol synthesis reactor is operated at a temperature above 190 degrees centigrade.

In one embodiment the methanol synthesis reactor is operated at a temperature below 400 degrees centigrade.

In one embodiment the hot methanol synthesis reactor exhaust gases are cooled or partially cooled via heat exchange against water or steam being fed to the reformer.

In one embodiment the hot or partially cooled methanol synthesis reactor exhaust gases are cooled via radiative heat exchange using air or other cool gases.

In one embodiment the hot or partially cooled methanol synthesis exhaust gases are cooled via a chiller system.

In one embodiment the moisture contained in the methanol synthesis reactor exhaust gases is partially or entirely condensed and removed.

In one embodiment the methanol is partially or entirely condensed and removed.

In one embodiment the methanol is distilled from the water in the synthesis reactor exhaust.

In one embodiment the dry product gas resulting from methanol synthesis is used directly as fuel or for other uses.

In one embodiment the dry product gas resulting from methanol synthesis is stored for later use.

In one embodiment tars present in condensate from oxygenated steam reforming are reduced or removed via aqueous catalytic oxidation over a copper or other suitable catalyst.

In one embodiment tars present in condensate from oxygenated steam reforming are reduced or removed via high-temperature oxidation in the absence of a catalyst.

In one embodiment condensate containing tars is heated using heat recovered from the reforming or methanation reactors.

In one embodiment condensate containing tars is heated using heat recovered from the reforming or methanation reactors and oxygen from electrolysis or other sources is used to oxidize tars to carbon dioxide and water.

In one embodiment condensate containing tars is directly recycled to the steam reformer.

In one embodiment condensate containing tars that is not recycled to the steam reformer is heated using indirect heat exchange from the reformer or methanation reactors and oxidized using oxygen from electrolysis or other sources.

In one embodiment heat is recouped from condensate containing tars that is not recycled to the steam reformer following oxidation treatment and that heat is used to preheat untreated condensate containing tars.

In one embodiment excess oxygen used to remove tars from condensate is fed to the reformer to provide the required amount for oxygenated steam reforming.

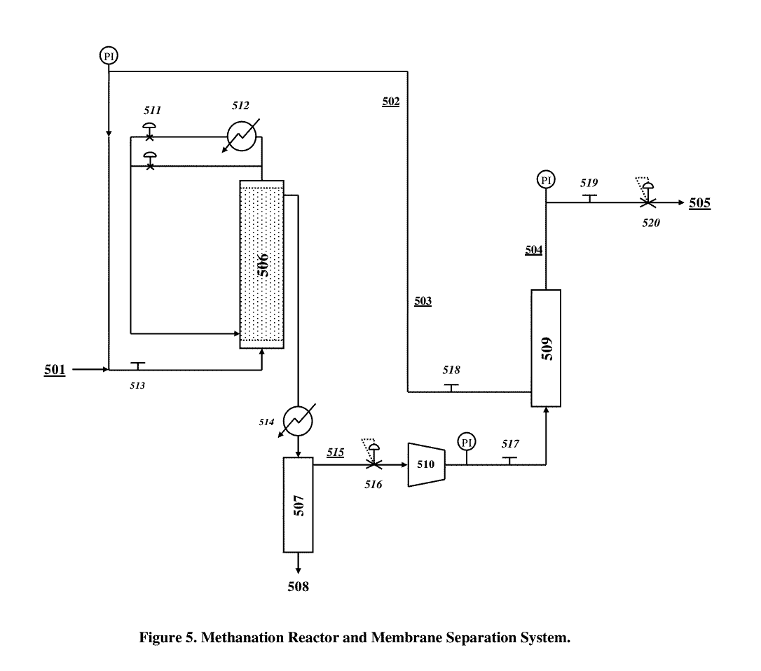

In one embodiment the methanation reactor feed gases are first passed through an indirect heat exchanger contained within the methanation reactor to carry away heat generated by the exothermic methanation reactions (see FIG. 5).

In one embodiment methanation feed gas heated by indirect heat exchange in the methanation reactor is cooled or partially cooled by radiative, convective, or conductive heat exchange.

In one embodiment methanation feed gas that has been passed through an internal reactor heat exchanger is fed to the methanation reactor catalyst bed while hot or after cooling.

In one embodiment the reformer gas is used as gaseous fuel for electricity generation either as-is or after removal of moisture.

In one embodiment the reformer gas is subjected to separations to remove non-fuel gases prior to use as a gaseous fuel for electricity generation.

### EXPERIMENTAL

### NOTE: Experiments are for Illustrative Purposes Only and should not be Considered Limiting

The following procedures may be employed for reforming and methanation of organic matter as described in the present invention. Key hardware for the production of methane and oxygen products from organic matter consists of an oxygenated steam reformer, a methanation reactor, and an electrolyzer as depicted in FIG. 2. Additional hardware includes condensers for removal of water from the reformer and methanation reactor exhaust gas streams, a gas compressor and membrane, and heat exchangers to maximize thermal efficiency. The following is based on the configuration of a system built and tested for treating organic wastes generated at a lunar exploration outpost in support of a human crew of four. The general procedures can be applied to virtually any organic feed matter on Earth or in space.

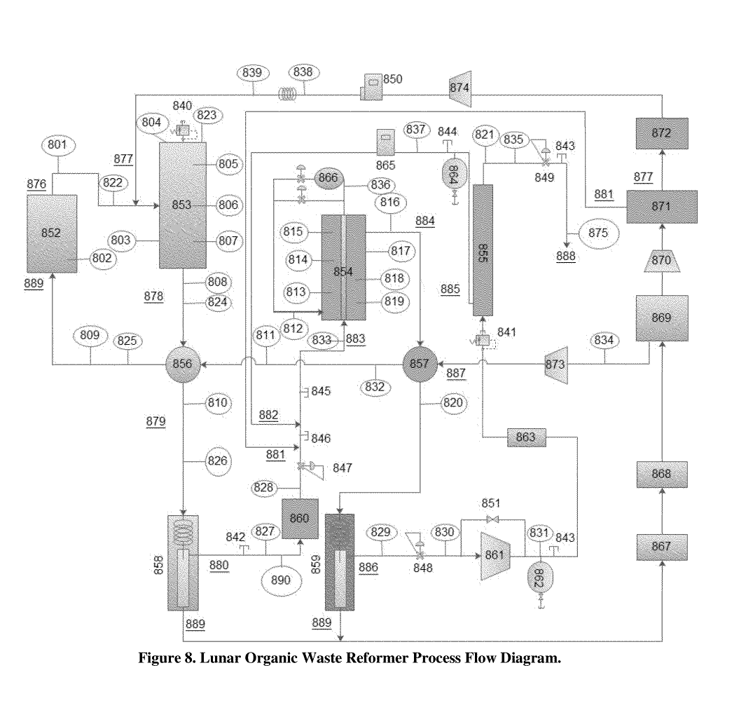

Shown in FIG. 8 is the overall process flow diagram for an oxygenated steam reforming and methanation system tailored to space applications hereby referred to as the Lunar Organic Waste Reformer (LOWR). Water from the reservoir is pumped into the steam generator, via two heat exchangers. Residual heat from the Sabatier methanation reactor and the steam reformer outlets are transferred to the water to reduce the amount of energy needed from electrical heaters to convert water to steam in the steam generator. Once exiting the generator, the steam is mixed with oxygen before entering the steam reformer filled with organic matter. The addition of oxygen produces an exothermic reaction that boosts the temperature within the reformer, which decreases the likelihood of tars and increases the reaction rate.

After the reformer, the product gas goes through the first condenser to remove water from the line, which is then recycled back to the water reservoir after first going through the water cleaning system. This water flows back into the boiler for steam generation or flows to the electrolyzer to decompose into oxygen and hydrogen. Some of this oxygen feeds into the steam inlet and the remaining oxygen is accumulated as product gas.

The generated hydrogen gas flows into the Sabatier reactor to react with the reformer output producing predominantly methane and water. The Sabatier product stream flows through the condensing system to remove the water. The water is sent through the cleaning system and either sent to the water reservoir for steam generator input or to the electrolyzer for separation into hydrogen and oxygen. The dried Sabatier product recycles through a membrane separator. Methane is a product and accumulates in a storage tank while hydrogen is recycled to the Sabatier reactor as needed. Methane can optionally be liquefied, which allows for further recovery and recycle of any hydrogen gas that may be present in the methane product. For the testing and demonstration described herein, the electrolyzer was simulated by the use of oxygen and hydrogen gases delivered from compressed gas cylinders with flows set at rates that would be produced by electrolysis to accommodate the integrated oxygenated steam reforming and methanation process.

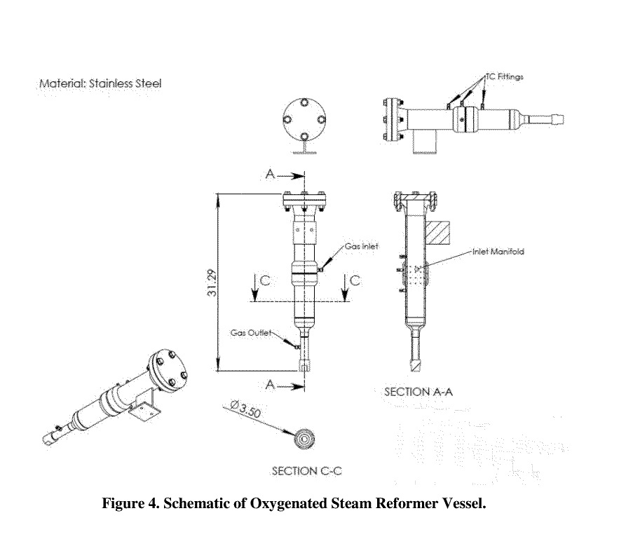

A key component of the LOWR is the steam reforming reactor. The reactor must be capable of handling a wide range of organic matter while operating in a consistent, reliable manner in order to integrate with other subsystems. The reactor design allows for occasional batch feed of organic matter while generating a relatively steady exhaust gas rate at relatively steady gas composition after steam injection is started. The reactor is of the approximate size required to accommodate at least ten kilograms of matter per day, and possibly significantly more. Most testing was carried out for periods of about two hours using organic matter pre-loaded into the feed magazine above the reforming zone of the reactor. For continuous or nearly continuous operation, a reactor similar to that described could be used with some additional provisions for repetitive, routine batch feeding. The down-flow oxygenated steam reformer is illustrated in FIG. 3. Additional design detail is shown in FIG. 4, with dimensions in inches. Organic matter is fed through the flange on the top of the reformer and flows down by gravity feed to be consumed in the reaction zone located in the lower portion near the steam-oxygen injection plenum.

A water injection pump was installed to deliver water to the oxygenated steam reformer via heat exchangers and a boiler. The pump had controls for both the stroke length and the number of strokes per minute. The stroke length was held constant and the strokes per minute were changed for the present application.

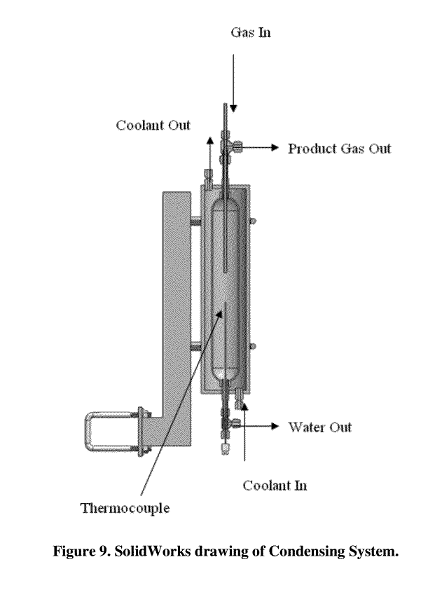

Product gases from the reformer pass through a condenser in order to remove water and recycle it. The condenser (FIG. 9) is comprised of an inner chamber surrounded by a coolant jacket. To control condenser temperatures, a chilling unit was chosen to circulate an ethylene glycol solution through the system. Gas enters the chamber through a quarter inch tube from the top of the vessel. The quarter inch tube is fitted into a tee, of which the other two ports are sized for ⅜^{th} inch tube. This allows space for the gas to exit the quarter inch tube and then make its way back up and out of the tee fitting. Upon reaching the cold zone inside the condenser, the steam is condensed to water and removed from the gas. The remaining gas then exits the chamber out of the top of the condenser. Water is periodically released from the inner chamber by a ball valve in the exit line at the base of the condenser.

Each part of the condenser was machined from 316 stainless steel. Downstream of both the biomass reformer and Sabatier methanation reactors are separate condenser modules that share a common two-stage ethylene glycol chiller system.

In order to insure that the oxygen input to the reformer cannot exceed an excessive amount that would lead to a thermal runaway condition, oxygen flow is limited by narrow-diameter tubing controlled by the pressure differential across the tubing. An oxygen pressure regulator controls oxygen flow rate through the narrow diameter tube. A LabView® control system was used for this application. However, any suitable automated or manual control system can be employed.

Extensive testing of the oxygenated steam reformer successfully reduced feeds of the type shown in the table below. Regardless of the organic matter feed content, the dry gas analysis and reformer residue content remained similar. Challenges such as feed bridging were resolved with a smaller feed size and a gravity plunger above the organic matter in the feed magazine.

Partial list of feeds used in steam reform testing

Feed
Charcoal
Wood Pellets
Potatoes
Dog refuse
Food Waste
Food Packaging
Diapers
Tape
Plastics

### Example 1

Shown in FIG. 14 is a typical temperature profile for the steam reformer during initial experiments performed in the reformer. For this test, 618 grams of potatoes and 393 grams of charcoal were used as feed. The steam reformer has five thermocouples that are monitored by a LabView® data acquisition and control system. One is located internally, below the reactor lid, which stays at 20° C. during the run. Another thermocouple is located on the outer shell of the reactor to control the pre-heater on the reactor, which is set at 400° C. The pre-heater is turned off before oxygen is introduced into the reformer. There are two thermocouples in the feed area of the reformer, one above the manifold where the gas is introduced and one below the manifold. There is a thermocouple located within the gas manifold. Lastly, there is a thermocouple located on the outlet of the reformer.

The test concludes when the outlet temperature exceeds the hottest temperature within the reformer. As shown, all three internal temperatures increase with the hottest zones at the gas inlet and the lower section of the reformer. As the feed is concentrated within the lower zone, its temperature rises above 900° C. requiring that the amount of oxygen fed to the reformer be reduced.

Before introduction into the steam generator, water is preheated with residual heat from the steam reformer exhaust. Shown in FIG. 15 is the temperature profile for the steam generator. The outlet temperature of the steam reaches 300° C. prior to mixing with oxygen.

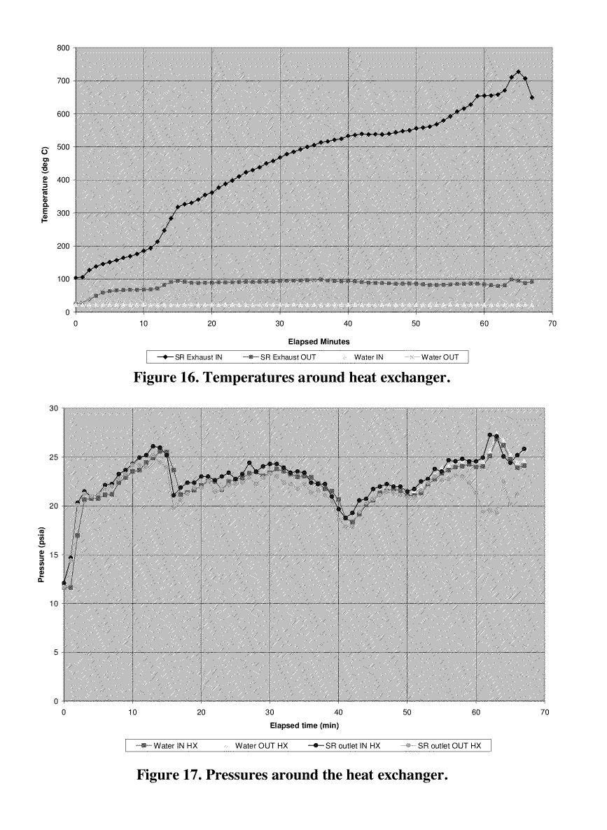

Shown in FIG. 16 is the temperature profile around the heat exchanger. The water partially turns to steam as the temperature exceeds 100° C. before it enters the steam generator.

Shown in FIG. 17 are the absolute pressures around the heat exchanger, which include the outlet pressure for the steam reformer and inlet pressure for the steam generator.

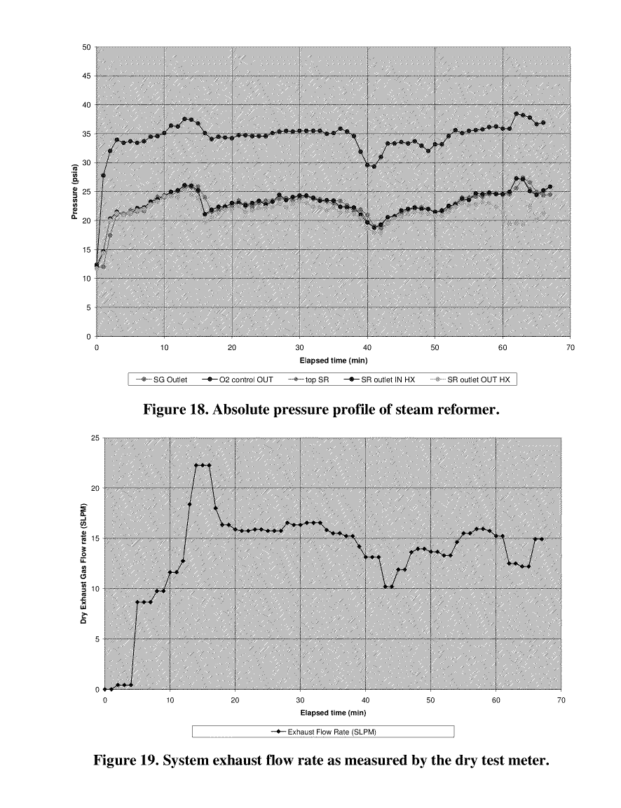

Shown in FIG. 18 is the pressure profile of the steam reformer including the oxygen-inlet control-pressure.

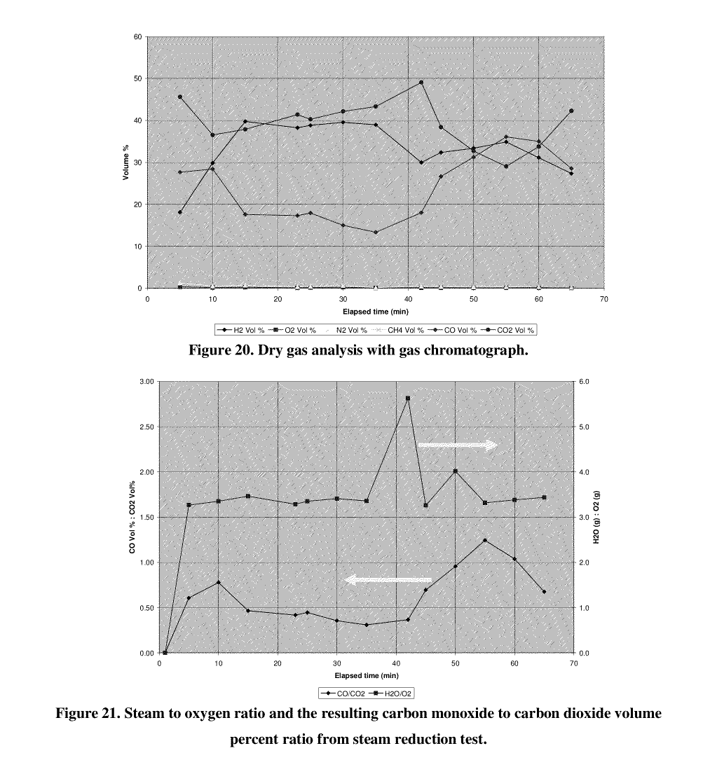

As the steam/oxygen mixture reforms the biomass, the resulting product gas predominantly contains hydrogen, carbon monoxide, carbon dioxide and water. After exchanging its heat with the inlet water through the heat exchanger, the exhaust gas enters the condensing system where the steam condenses and is removed from the exhaust gas. The dry gas then passed through a dry test meter, where the flow rate was measured as shown in FIG. 19. It then passed through a ZnO sulfur trap and a gas chromatograph port for analysis. The dry gas analysis is overlaid on the steam-reformer temperature profile and shown in FIG. 20.

Shown in FIG. 21 are the steam to oxygen ratio and the resulting carbon monoxide to carbon dioxide volume-percent-ratio. The steam to oxygen ratio increases as the oxygen flow rate is lowered when the maximum material temperature of 900° C. is reached. As a result of the increase of steam in the reactor, the carbon monoxide to carbon dioxide ratio also increases.

Remains after oxygenated steam included ash, charcoal/potato char and the remaining alumina felt used as a filter. The ash was less than ten percent of the feed and the remaining char can be used for the start of a subsequent test. After this test, the alumina felt was eliminated. Instead, the feed was directly placed on top of a zirconia foam filter.

A zinc oxide bed was installed to capture sulfur in the reformer system exhaust. H₂S was found to be the predominant sulfur contaminant along with smaller amounts of COS. The ZnO bed removed virtually all of the H₂S in the dry reformer exhaust but did not remove the COS. Other sorbents tailored to the application can be used to remove gaseous sulfur, chlorine, fluorine and other contaminants.

### Example 2

A key component of the present invention is the methanation or Sabatier reactor. Based on previous experience, it was determined that a reactor volume of 500 cm³ would be sufficient for the methanation application described herein and as illustrated in FIG. 6. Much of the required hydrogen for the Sabatier reaction is supplied with the reformer exhaust gas. The exact amount of available hydrogen depends on the reformer feed composition and reformer operating conditions. Additional hydrogen is provided by electrolysis while co-producing the oxygen.

A piece of two-inch diameter 316L stainless steel was chosen for the reactor body. The tube was cut to approximately 12 inches long. The reactor body was drilled to provide four thermocouple ports and a larger port to fill/empty the catalyst. Heaters were wrapped around the lower and upper sections of the reactor, allowing for different temperatures to facilitate start-up and routine operations. The reactor was insulated to minimize heat loss to the surroundings.

The methanation reactor is filled with catalyst (Johnson Matthey 0.5 percent Ruthenium concentration on three-millimeter alumina tablets) through a side port. The bulk density of the catalyst as loaded into the reactor is about one kg/L.

Sabatier methanation reactions shown above in equations (7) and (8) can be carried out in the range of 350 to 500° C. at about one bar pressure absolute to provide nearly complete conversion to methane with minimal production of carbon when using an excess of hydrogen approximately 25 percent over the stoichiometric requirements.

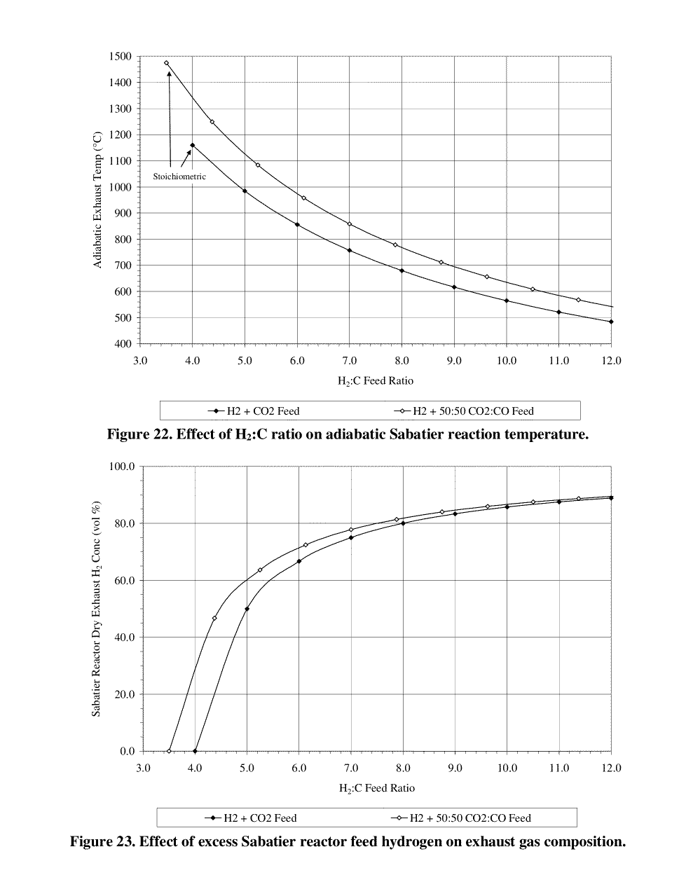

Thermal management to prevent overheating of the catalyst is an important consideration in the design of the reactor due to the exothermic nature of these reactions. FIG. 22 shows the adiabatic temperature of methanation reactions as a function of hydrogen stoichiometry. Adiabatic reaction temperatures of over 1100° C. result from conversion of carbon dioxide and carbon dioxide plus carbon monoxide to methane when hydrogen is supplied at its stoichiometric ratio to provide complete conversion. If allowed to reach high temperatures (above 600° C.), conversion is reduced due to decreasing equilibrium constant at higher temperatures. Therefore, means to control methanation reaction temperature are important.

FIG. 22 shows that increasing the H₂:C ratio in the feed gas can significantly reduce the adiabatic temperature (while also providing a driving force for greater carbon conversion). FIG. 23 illustrates the effect of increasing the H₂:C ratio in the feed gas on the hydrogen gas concentration in the methanation reactor exhaust, assuming complete conversion of carbon oxide gases to methane.

The use of excess hydrogen during the methanation reaction is one of the approaches taken in the present invention to achieve full conversion of carbon oxide gases to methane.

An additional approach to control temperatures in the methanation reactor taken in the present invention is to use indirect heat exchange within the catalytic methanation reactor. The cool inlet gas to the methanation reactor was fed through a co-current indirect heat exchanger, which was configured as a single-pass along the methanation reactor center line. Additional passes could be used for larger reactors or reactors of different dimensions. The reactor configuration is shown in FIG. 6 and is shown integrated with the subsequent water condenser, recycle pump, and membrane separator in FIG. 5. The inlet gas is heated as it flows through the indirect heat exchanger. The indirect heat exchanger could be configured for counter-current or co-current flow. However, a co-current orientation was most effective for the methanation reactions employed in the present invention. Heat recovered in the methanation feed gas by indirect heat exchange can be rejected downstream of the reactor in a controlled fashion via external cooling as shown in FIG. 6. The methanation feed gas temperature is adjusted to promote initiation of the exothermic reaction without quenching or overheating.

The Sabatier methanation reactor, condenser, recycle pump, and gas separation membrane were configured as shown in FIG. 5. The dried methanation reactor exhaust gas was compressed using a KNF UN022 series diaphragm pump and fed to a gas separation membrane. Hydrogen and carbon dioxide diffuse through the Permea polysulfone membrane (PA1020-PI-2A-00) at a much greater rate than methane. Therefore, the pump and membrane separator enable excess hydrogen to be recycled to the Sabatier reactor to provide favorable thermodynamics for complete conversion of CO and CO₂ to CH₄ and to moderate the reaction exotherms while producing a high-purity methane product. The methanation reactor exhaust gas recycle system also provides a measure of process stability by presenting an opportunity for un-reacted CO₂ to be returned to the Sabatier reactor along with the excess hydrogen. A backpressure regulator was installed on the membrane retentate stream as the primary operating control for separations at given flow rates and feed gas compositions. The retentate (methane-rich gas) backpressure was set as required (between 20 and 50 pounds per square inch gauge pressure, although higher pressures can be used for even greater performance). The permeate (hydrogen and carbon dioxide-rich gas) discharged to atmospheric pressure.

Experiments conducted using compressed hydrogen, carbon dioxide, and carbon monoxide gases in the configuration shown in FIG. 5 produced nearly complete conversion of carbon-containing gases to methane. The simulated dry reformer exhaust gases were metered through mass flow controllers. Hydrogen was fed at a rate of approximately 13.4 SLPM H₂ (representing the reformer exhaust of 4.5 SLPM plus the projected 8.9 SLPM from electrolysis and recycle from the methane product after liquefaction of CH₄). Carbon monoxide and carbon dioxide were metered at rates of about 1.7 and 2.1 SLPM, respectively, to represent the projected reformation product gas flows. The gases fed from the cylinders were mixed with the recycled membrane permeate as shown in FIG. 5. Gas samples were taken at the Sabatier methanation feed under each experimental condition and were analyzed in a Varian CP-4900 micro gas chromatograph. The membrane permeate was mixed with the simulated reformer exhaust gas just upstream of the Sabatier reactor. The Sabatier reactor feed gas was first passed through the internal, indirect heat exchange tube to remove heat from the system. After cooling in a heat exchanger, the feed gas was then fed to the catalyst bed in an up-flow configuration as shown in FIG. 5. The heat control valves were adjusted during operations to provide a Sabatier inlet gas temperature sufficient to avoid quenching at the reactor inlet.

The Sabatier reactor was operated at temperatures between 400 and 500° C. The heat removal provided by a single pass of cold feed gas was just sufficient for the target feed rates used during this set of experiments—any additional inlet flow of CO and/or CO₂ would have resulted in temperatures that would be too high to achieve complete conversion of carbon oxides to methane at reasonable excess hydrogen concentrations.

The Sabatier exhaust gas was passed through a heat exchanger normally used to preheat water used for reforming After further cooling in a chiller, the condensed water was removed from the Sabatier methanation exhaust gas. The dry Sabatier exhaust gas was fed to the suction of the gas recycle pump, which directed the flow to the separation membrane. A backpressure regulator just upstream of the pump was set to maintain the Sabatier reactor and permeate at just above atmospheric pressure. In a system fully integrated with a reformer, this would allow introduction of the reformer gas at low pressure while keeping the Sabatier reactor from operating under reduced pressure. A backpressure regulator on the membrane retentate (methane product) was set at different values during the experiments to gauge the effect on system performance. The following table summarizes results obtained using the methanation reactor, condenser, recycle pump, and separation membrane described above.

Effect of Gas Separation Retentate Pressure

| | | | | | |
|---|---|---|---|---|---|
| Process | Vol % | Vol % | Vol % | Vol % | Vol % |
| Stream | H₂ | CO | CO₂ | CH₄ | Total |

Retentate Pressure = 20 psig (14:23)

| | | | | | |
|---|---|---|---|---|---|
| Sabatier Feed | 75.94 | 8.56 | 13.30 | 2.21 | 100.00 |
| Sabatier Exhaust (Dry) | 52.11 | 0.00 | 5.83 | 42.06 | 100.00 |
| Permeate | 73.81 | 0.00 | 9.74 | 16.46 | 100.00 |
| Retentate | 31.66 | 0.00 | 5.16 | 63.18 | 100.00 |

Retentate Pressure = 30 psig (13:58)

| | | | | | |
|---|---|---|---|---|---|
| Sabatier Feed | 75.91 | 8.29 | 12.40 | 3.40 | 100.00 |
| Sabatier Exhaust (Dry) | 59.13 | 0.00 | 2.75 | 38.12 | 100.00 |
| Permeate | 78.76 | 0.00 | 3.65 | 17.59 | 100.00 |
| Retentate | 20.33 | 0.00 | 0.76 | 78.90 | 100.00 |

Retentate Pressure = 40 psig (14:53)

| | | | | | |
|---|---|---|---|---|---|
| Sabatier Feed | 77.35 | 7.57 | 11.10 | 3.98 | 100.00 |
| Sabatier Exhaust (Dry) | 56.85 | 0.00 | 2.03 | 41.13 | 100.00 |
| Permeate | 79.81 | 0.00 | 2.16 | 18.03 | 100.00 |
| Retentate | 16.28 | 0.00 | 0.78 | 82.93 | 100.00 |

Retentate Pressure = 50 psig (15:34)

| | | | | | |
|---|---|---|---|---|---|
| Sabatier Feed | 78.53 | 7.02 | 9.69 | 4.76 | 100.00 |
| Sabatier Exhaust (Dry) | 62.02 | 0.00 | 0.83 | 37.15 | 100.00 |
| Permeate | 83.08 | 0.00 | 0.24 | 16.68 | 100.00 |
| Retentate | 14.22 | 0.00 | 0.10 | 85.68 | 100.00 |

Retentate Pressure = 50 psig (repeat) (16:02)

| | | | | | |
|---|---|---|---|---|---|
| Sabatier Feed | 78.52 | 7.03 | 9.63 | 4.82 | 100.00 |
| Sabatier Exhaust (Dry) | 62.38 | 0.00 | 0.56 | 37.06 | 100.00 |
| Permeate | 82.11 | 0.00 | 0.19 | 17.70 | 100.00 |
| Retentate | 13.50 | 0.00 | 0.07 | 86.43 | 100.00 |

One key result from the experiments was that carbon monoxide was entirely converted in the Sabatier reactor under all of the operating conditions. In addition, no evidence of coking was detected in the Sabatier reactor. The overall results of the analyses obtained by gas chromatography show that higher retentate operating pressures result in higher purity of the methane product stream.

Results from the integrated Sabatier-membrane-recycle pump system were summarized to show the per-pass conversion of CO plus CO₂ to methane (based on the Sabatier exhaust gas composition) and the overall system conversion to methane (based on the methane product gas composition in the membrane retentate). The amount of excess hydrogen supplied to the Sabatier reactor as a result of the hydrogen-rich permeate recycle gas was also calculated.

Effect of Membrane Retentate Pressure
on Sabatier System Performance.

| Retentate | Stoichiometric | Per-Pass | System |
|---|---|---|---|
| Pressure | Hydrogen | Conversion | Conversion |
| (psig) | Excess | (%) | (%) |
| 20 | 1.04 | 87.8 | 92.5 |
| 30 | 1.02 | 93.3 | 99.0 |
| 40 | 1.15 | 95.3 | 99.1 |
| 50 | 1.31 | 97.8 | 99.9 |
| 50 (repeat) | 1.32 | 98.5 | 99.9 |

Notes:
Target inlet flows = 13.4 SLPM H₂, 1.7 SLPM CO, and 2.1 SLPM CO₂
Constant permeate pressure of about 2 psi
Hydrogen excess calculated from Sabatier feed gas composition
Per-pass conversion calculated from Sabatier exhaust composition
System conversion calculated from methane product composition

A more-detailed analysis of the results was prepared for the 50-psi retentate operating point. The flow measurements and gas chromatograph (GC) results for the 50-psi repeat experiment were projected to a material balance that includes H₂, CO, CO₂, and CH₄ as well as H₂O produced in the Sabatier reactor. The material balance is based primarily on the measured retentate flow rate and GC gas compositions. The CO and CO₂ input flow rates were proportioned to match the total carbon throughput—the measured retentate flow of CO₂ plus CH₄ was about 95 percent of the input target rate of CO plus CO₂. Therefore, the CO and CO₂ were assigned flows of 1.6 and 2.0 SLPM, respectively, instead of the target flows of 1.7 and 2.1 SLPM. In general, actual GC analyses of intermediate process streams were used in the material balance, and intermediate process stream flow rates were calculated from the measured or assigned inlet and retentate flow rates. The material balance does not fully close due to flow measurement and analytical uncertainties. However, the balance provides a good preview of performance that could be expected under conditions representative of a fully integrated reformer with the Sabatier-membrane-recycle system. FIG. 24 shows the material balance for the Sabatier-membrane-recycle system operating at a retentate pressure of 50 psig. Results from the integrated Sabatier-membrane-recycle system experiments were used to guide subsequent integrated oxygenated steam reformer-methanation operations, in which dry reformer exhaust gas was fed to the Sabatier system.

### Example 3

Integrated oxygenated steam reforming and methanation experiments were conducted according to the flow sheet shown in FIG. 2. Except for electrolysis, the LOWR was operated in an entirely integrated mode during these experiments. The oxygen requirement for reforming and the hydrogen requirement for the Sabatier system were provided using compressed gas cylinders rather than an electrolyzer. In practice, the electrolyzer production rate would be adjusted to provide the required hydrogen flow for the Sabatier reactor while simultaneously providing oxygen for reforming plus excess oxygen as product.

For the initial integrated experiment, feed consisting of potatoes (with charcoal as a startup material) was chosen to facilitate reformer operations with a familiar performance profile.

Previous reformer experiments were carried out using potato feed with oxygen rates between 3.1 and 3.8 standard liters per minute at a steam-to-oxygen ratio of about 3:1. Average dry reformer gas rates on the order of 12 SLPM were produced during these experiments. This is about double the flow that the current Sabatier reactor configuration can handle while still providing effective thermal management. Therefore, the inputs of oxygen and steam to the reformer were cut to about one-half of those used during earlier experiments. The overall rates in the reformer and Sabatier under these conditions (1.6 SLPM 02 and 2.3 grams H₂O per minute) approximate system operations with about 100 percent on-stream factor to accommodate waste from a space outpost crew of four.

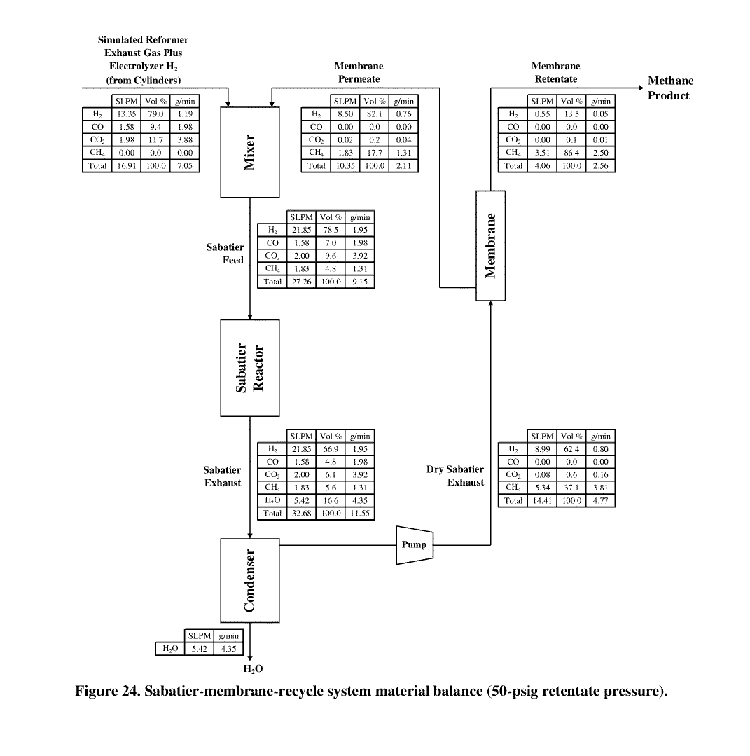

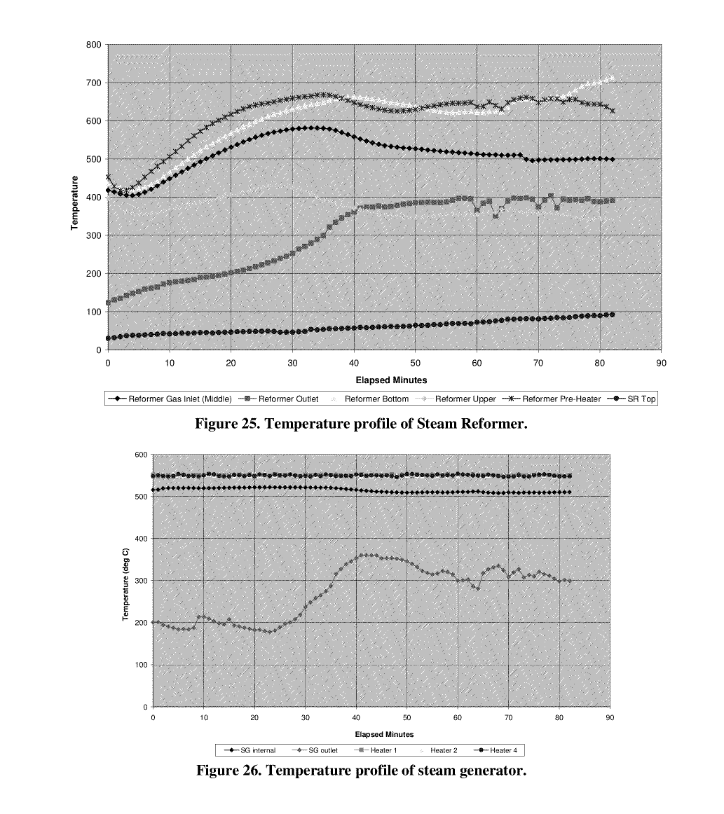

Shown in FIG. 25 is the temperature profile of the reformer as oxygen is introduced and then through the integrated run.

Shown in FIG. 26 is the temperature profile in the steam generator during the startup of the system and through the integrated run.

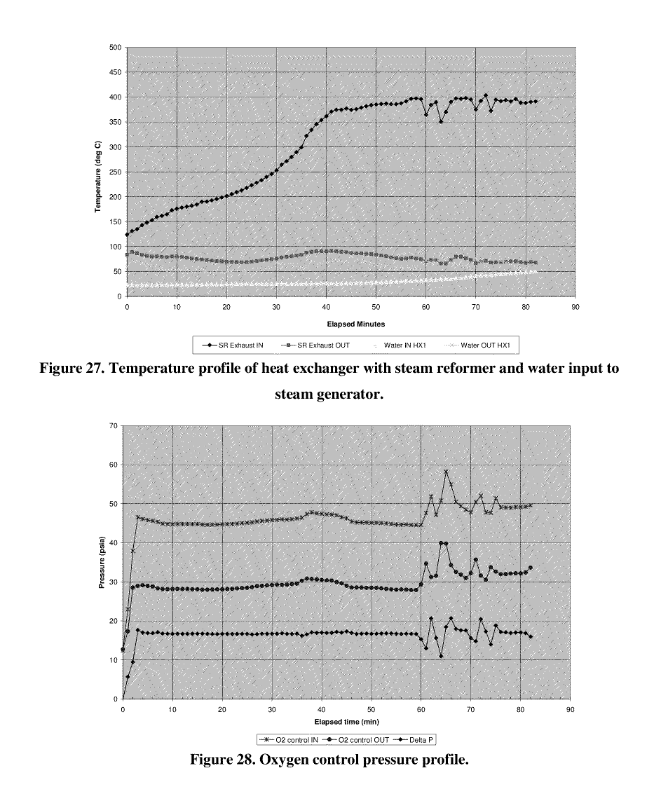

Shown in FIG. 27 is the temperature profile of the heat exchanger in which the reformer exhaust gas transfer heat to the water input into the steam generator. All profiles are similar to ones seen in previous tests where Block 1 was tested separately.

Shown in FIG. 28 is the pressure profile of the oxygen control system. A forward pressure regulator is used to maintain a constant pressure to insure constant oxygen flow rates.

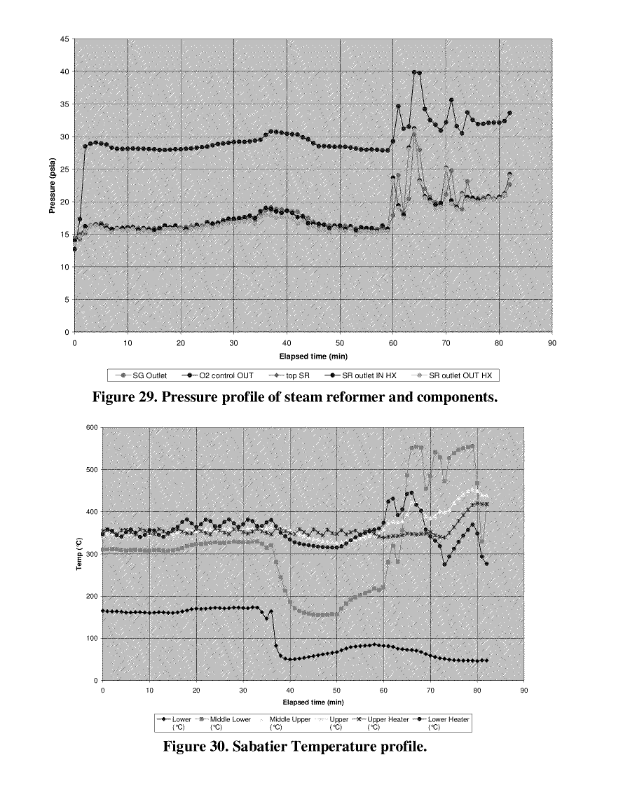

Shown in FIG. 29 is the pressure profile around the steam reformer and the heat exchanger through which the reformer exhaust gas flows to transfer heat to the water input into the steam generator.

At the oxygen and steam rates selected for the first integrated operation, the dry reformer exhaust rate was somewhat greater than projected. As a result, the total CO plus CO₂ flow rates from the reformer were about 4.5 SLPM rather than the targeted rate of about 3.6 SLPM (not including hydrogen). The higher CO plus CO₂ rates generated somewhat more heat in the Sabatier reactor than could be readily accommodated with the existing reactor configuration. Because the reformer exhaust gas rate was greater than anticipated, the pre-set flow of hydrogen that simulated the electrolyzer output was lower than needed to provide complete conversion of CO plus CO₂ to CH₄. The lower hydrogen rate also limited heat dilution in the Sabatier reactor by reducing the amount of hydrogen available for recycle. Because the carbon oxide conversion (and associated reduction of gas volume) was lower than anticipated, the target 50-psi retentate operating pressure could not be achieved. Consequently, the membrane-recycle system was operated at a retentate pressure of 30 psi, which increases the amount of hydrogen carried with the methane product. Despite the somewhat off-nominal operating conditions, the first integrated experiment resulted in an overall process conversion of 89.7 percent of the carbon oxides generated during reforming into methane at a purity of 81.6 volume percent. The conversion is based on the analysis of the membrane retentate (methane product).

Shown in FIG. 30 is the temperature profile in the Sabatier reactor during startup and through the integrated run.

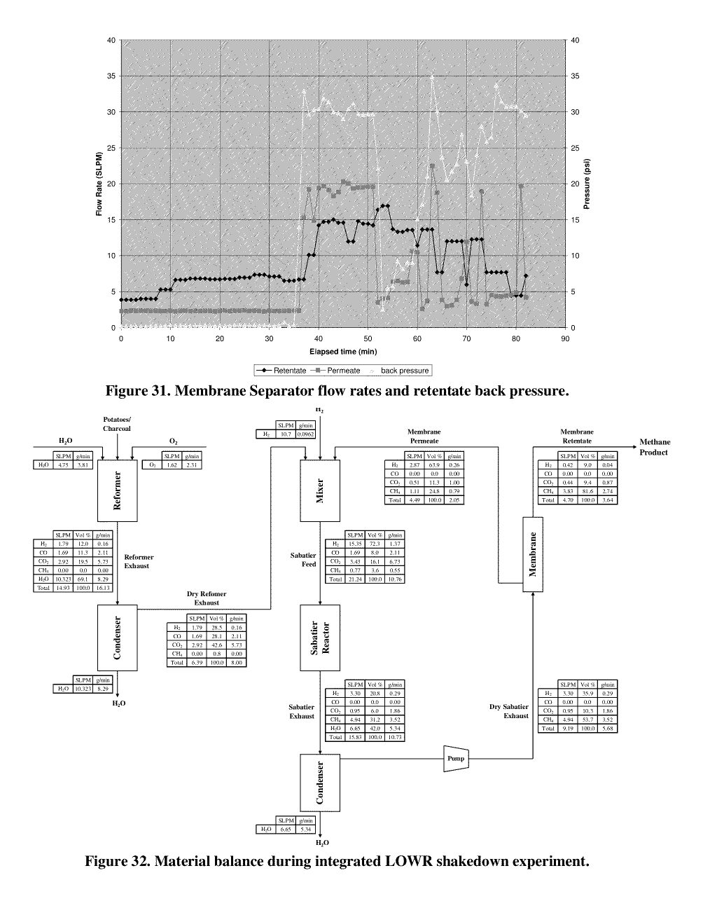

Shown in FIG. 31 are the flow rates from the permeate and retentate exhaust flows from the membrane separator. Also shown is the backpressure maintained on the retentate exhaust line.

FIG. 32 shows a material balance that was prepared for the integrated Block 1 and Block 2 LOWR systems. The balance is based primarily on the measured gas compositions and retentate flow rate shown in the figure. Intermediate process flow rates were generally back calculated from the gas compositions and known down-stream flow rates. As a result of analytical and measurement uncertainties, the balance around each block may not be perfect. However, alternate calculations (using a hydrogen balance rather than a methane balance, for example) showed that most calculated values were consistent with measured values shown in the material balance. The total carbon throughput during the integrated operating period was on the order of 3.5 kg per day (rather than a rate of about 2.8 kg per day for a high-fidelity feed derived from a crew of four). Therefore, the system was operating a rate of about 40 percent more than required (assuming a 100% on-stream factor).

The actual hydrogen feed rate (which represents the amount of hydrogen required to supplement hydrogen contained in the reformer exhaust to achieve complete conversion of carbon oxides in the Sabatier reactor) was determined to be about 10.6 SLPM instead of the targeted 11.7 SLPM. This, in combination with the higher-than-expected reformer carbon output, resulted in the hydrogen deficiency noted above. Another adjustment to the measured or target material balance values was that the CO₂ concentration of the membrane feed (dry Sabatier exhaust) had to have been about 10.3 percent rather than the 6.7 percent measured by the GC in order to obtain the concentrations measured in the permeate and retentate. This correction was performed in a manner that resulted in the most conservative performance estimate (i.e. lower overall system conversion). One other minor adjustment to the material balance was to disregard the small amount of methane generated in the reformer. This improved the balance around the Sabatier reactor as well as the overall balance but did not appreciably change key performance factors.

One key observation made during all Sabatier operations is that carbon monoxide is entirely converted in the Sabatier reactor. This is important for the LOWR because unreacted CO is much more likely to report to the membrane retentate (methane product) and would therefore not be recycled to the Sabatier reactor via the permeate.

### Example 4

Results from the initial LOWR integration experiment led to revisions in operating procedures that enabled quicker startup and Block 1 and Block 2 system integration. Evaluation of the experiment also led to identification of relatively simple feedback controls. Testing of the integrated Block 1 and Block 2 systems continued. Work centered on refining the operating methods and on establishing a control scheme for the Sabatier system while gathering further performance data over a range of conditions.

In a follow-up to the initial integrated operations reported previously, the Block 1 and Block 2 systems were operated using potatoes as feed to the reformer. Infrared-based methane and carbon dioxide sensors were installed at the Block 2 exhaust (membrane retentate) to provide real-time analyses to aid control of the system. Because carbon monoxide has been observed to be completely converted in the Sabatier system, the system exhaust gas contains only methane, hydrogen, and carbon dioxide. Under nominal operating conditions, only small amounts of CO₂ are present in a gas stream containing mostly CH₄ with small amounts of excess H₂ used to drive the Sabatier reaction to near completion. Therefore, the complete Block 2 exhaust stream analysis can be obtained by direct measurement of CH₄ and CO₂ with H₂ determined by difference.

In principal, the hydrogen fed to the Sabatier system to supplement hydrogen already present in the reformer exhaust can be adjusted based on the exhaust gas sensors. Under a condition in which CO₂ is zero or very low and CH₄ is observed to be dropping, it can be assumed that H₂ is being fed at more than its required rate. Conversely, if CO₂ is present in the Block 2 exhaust and is observed to be rising, it can be assumed that H₂ is being fed at less than its required rate. The system was operated with consideration of these scenarios to determine if a relatively simple feedback control could be applied. One goal of the effort was to establish nominal sensor readings that correspond to high carbon conversions to methane with minimal excess hydrogen in the methane product.

During the integrated operation, the infrared sensor values were compared to gas chromatograph analyses, and the trends observed were used to make manual adjustments to the hydrogen flow in order to simulate a feedback control system. During the course of the integrated run, the system exhaust gas typically contained between four and 15 volume percent CO₂ (representing conversions of carbon to methane between 83 and 95 percent). Results of the on-line methane sensor (spanned to show zero to 100 percent CH₄) were verified to be quite close to those obtained by gas chromatography. A full range (zero to 100 percent CO₂ span) carbon dioxide sensor was chosen to allow for full characterization of the methane product gas stream. It was recognized that the calibration for CO₂ might not be precise at the low values targeted for LOWR operations. However, the primary duty of the sensor is to detect when CO₂ is present and whether the amount is steady, increasing, or decreasing. As expected, operating results showed that the carbon dioxide sensor tended to exaggerate CO₂ values somewhat at lower concentrations. This actually helped to amplify small changes in exhaust gas composition, leading to a potentially quicker control response.

Some fluctuations/oscillations in membrane permeate flow rate, recycle pump inlet pressure, and reformer pressure were noted during this initial integrated run using the on-line exhaust gas sensors. The initial operating philosophy was to try to maintain a significant hydrogen excess to push the Sabatier reaction to near completion. Any serious amount of excess hydrogen would simply report to the membrane retentate along with the methane product and would therefore not significantly impact other system operations. However, the operating data showed that under conditions of excessive hydrogen feed rate, system fluctuations occurred as a result of increasing hydrogen volume (and an associated pressure increase) in the membrane permeate and process lines leading to the recycle pump inlet. Because hydrogen has such a strong propensity to permeate through the membrane, recycle system pressure built up before all of the excess hydrogen began to report to the retentate. This build-up in system pressure caused the recycle pump inlet pressure to build up and to exceed the pressure of the dry reformer exhaust (nominally less than five psig), resulting in an accumulation of reformer product in Block 1. As a result, the Sabatier system almost completely filled with hydrogen. As the Sabatier system stabilized in this condition, the reformer built pressure as oxygen and steam flows continued to make fresh product. When the reformer pressure exceeded the Sabatier pressure, the carbon oxide reforming gases began to enter the Sabatier reactor where they were quickly converted to methane, resulting in a significant reduction of system volume (by consumption of multiple hydrogen molecules) to convert CO and CO₂. This lowered the Sabatier system pressure and allowed a further in-rush of dry reformer gas, which consumed the excess hydrogen and resulted in lower conversions to methane. With a constant flow rate of fresh hydrogen to the Sabatier reactor, the cycle described above continued. It was noted that the rate of these oscillations decreased as the flow rate of hydrogen was decreased. Significant lessons about the effects of hydrogen flow rate (representing electrolysis output) and the required adjustment steps useful for process control were obtained from the integrated run. Despite the significant deviations from nominal target conditions, the overall conversion of reformer CO and CO₂ to CH₄ typically remained greater than 85 percent.

### Example 5

Prior to the next integrated run, some revisions to system settings and operating procedures were made in order to reduce process fluctuations and to boost carbon conversion. One key change was to set the reformer backpressure regulator to ten psig rather than the nominal two psig used previously. This facilitated a continuous flow of reformer gas to the Sabatier system (or at least allowed greater time to respond to make hydrogen flow adjustments before Sabatier recycle pump inlet pressure exceeded the ten psig reformer pressure) even if Sabatier pressures fluctuated as described above.

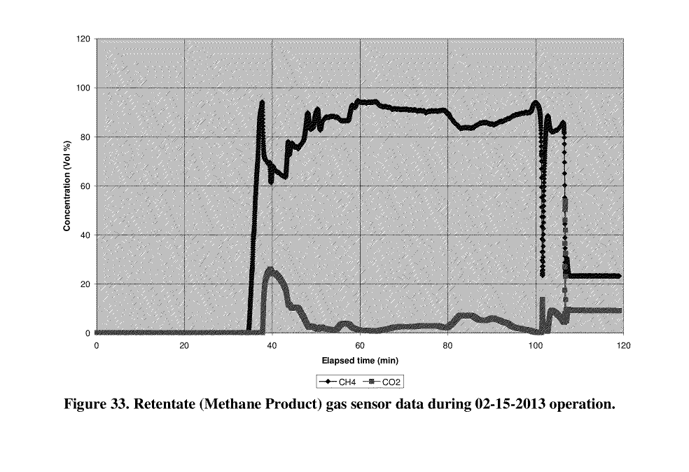

The next run used potatoes as reformer feed (above a bed of charcoal in the reformer exhaust) as before. The integrated process run exhibited significantly improved stability as a result of the increased reformer backpressure, closer match of Sabatier hydrogen flow rate to the required amount, and somewhat quicker response to observed exhaust gas concentrations. Although some instabilities of the type detailed above were noted, the extent and duration were greatly reduced. As needed, the Block 2 retentate pressure was briefly reduced to vent excess gas to return to stable operation as the primary control parameter (hydrogen flow rate to the Sabatier system) was adjusted. After making hydrogen flow rate adjustments early in the run, a relatively steady retentate (methane product) gas composition was obtained. Some variation in product gas concentrations occurred as a result of slightly changing reformer flow and composition and the resulting changes in hydrogen flow rate to the Sabatier reactor. FIG. 33 shows the on-line methane and carbon dioxide analyses during the run. Note that the carbon dioxide concentrations are somewhat over-reported; however, the trends were the primary input to controlling the process.

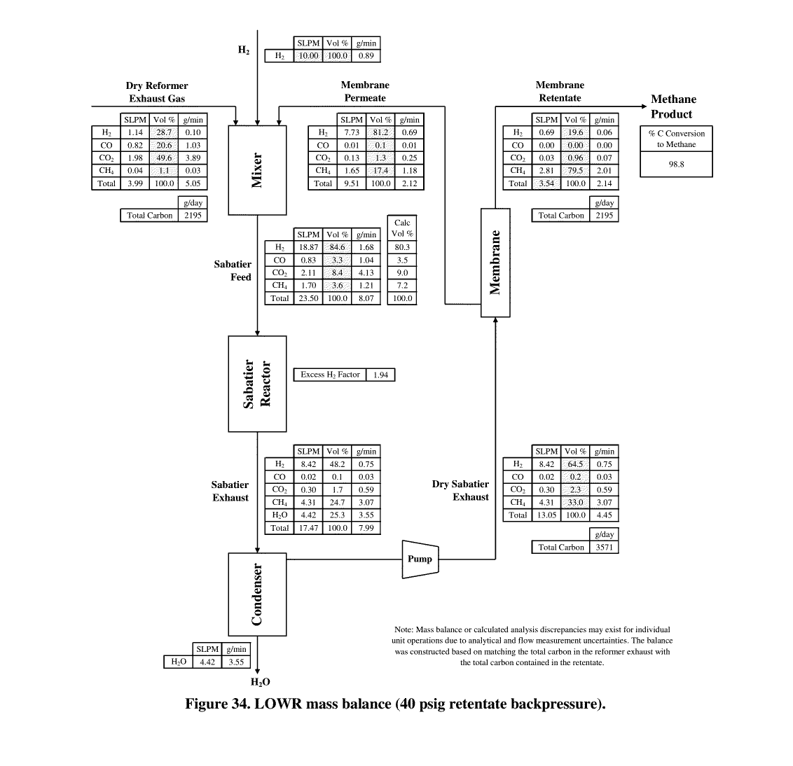

During a steady operating period, a complete set of gas analyses and flow rates were taken from the integrated Block 1 and Block 2 system as a snapshot of a steady-state operation. During this period, the reformer and Sabatier systems were processing about 2.2 kg of carbon per day into methane, which is on the order of the requirements for a full-scale LOWR operating continuously for a lunar outpost application. Gas analyses included the dry reformer exhaust, the Sabatier reactor inlet, the dry Sabatier reactor exhaust, the membrane permeate, and the membrane retentate (methane product). The retentate flow rate and the hydrogen addition flow rate to the Sabatier reactor were also recorded. These gas analyses and flow rates were the basis of the material balance shown in FIG. 34. The hydrogen analyses were used to calculate unmeasured flows. (Results using methane analyses to calculate flows were generally found to be very similar to those using hydrogen). As noted in the figure, some discrepancies occurred due to analytical and flow uncertainties. However, the balance represents the overall operation quite well.

Results of the steady operating period during the run showed a conversion of carbon oxides to methane of 98.8 percent. No CO was detected in the methane product, and only about one percent CO₂ was present. The CO₂ could be collected on a molecular sieve for recovery and return to the Block 2 Sabatier section of the process. The relatively high excess hydrogen content (about 1.9× stoichiometric) resulted in a hydrogen concentration of about 20 volume percent in the methane product. This hydrogen could be collected during methane liquefaction and returned to the process.

### Example 6

Based on the improvements noted during this run, additional integrated operations were planned and carried out to further refine the overall system operations and controls. The changes were in part directed toward facilitating integration with minimal upsets. For example, the reformer back pressure was increased to its 10 psig operating point prior to integration into a Sabatier system operating at low retentate pressure at the desired initial hydrogen flow rate (which was estimated to be a close match to the required steady state value) with the recycle pump running Upon integration of Block 1 to Block 2 (by directing the reformer exhaust to the Sabatier system feed instead of venting), the retentate pressure was quickly increased to its target 30 to 50 psig. (The retentate pressure was set to the highest value possible within the current draw limit of its motor.)

These modifications were placed in effect for the next integrated run. After start up and integration, conversion of carbon oxides to methane of about 99.5 percent was obtained based on gas chromatograph analysis of the retentate (78.2 percent methane and 0.39 percent carbon dioxide with balance hydrogen). After less than an hour of integrated operation, the oxygen sensor on the reformer exhaust began to show a quick, steady increase in oxygen concentration to about five percent (versus typical values of less than 0.1 percent). Although no other symptoms of process upset were noted, operating safety protocol required a system shutdown, which was promptly performed. Subsequent evaluation and calibration showed that the reformer oxygen sensor had failed (gas chromatograph analyses showed less than 0.1 percent sulfur, but these have a lag time of about five minutes). The electrochemical oxygen sensor was replaced prior to the next run.

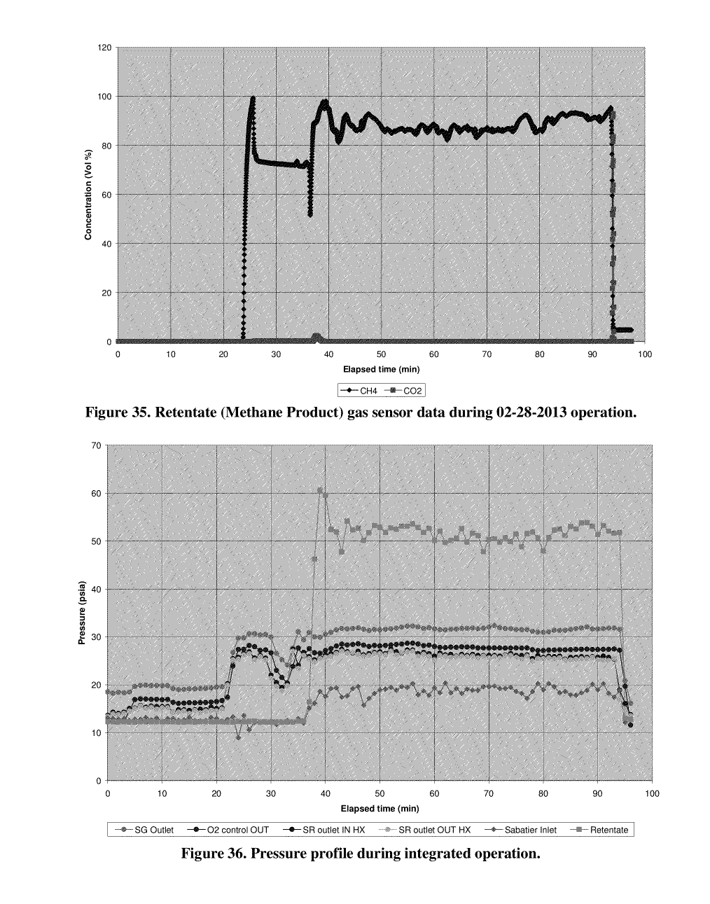

Integrated operations continued with the same goals as those of the previous run that was suspended due to the reformer oxygen sensor fault. Measures taken to adjust the Sabatier reactor hydrogen feed flow rate based on the on-line retentate analyses of CH₄ and CO₂ were largely effective in reducing the variability of flows, pressures, and gas compositions in the Block 2 system. FIG. 35 shows the CH₄ and CO₂ retentate sensor readings during the integrated operation. After an initial process excursion (due to selection of an initially high hydrogen flow rate to the Sabatier reactor), the remainder of the integrated operation was steady with respect to conversion of carbon oxides to methane.

There were still a few pressure fluctuations requiring short-duration venting of gas from the Block 2 unit (by reducing the retentate pressure briefly). These resulted from gradual hydrogen build up in the system that was not addressed quickly enough by reducing hydrogen flow rate. The Block 1 and Block 2 pressure profiles are shown in FIG. 36. As seen in the figure, pressure fluctuations were subtle and based on the product gas analyses had minimal effect on operations.

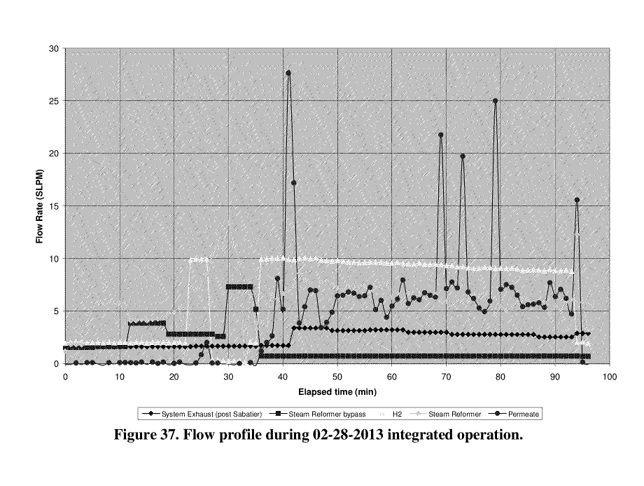

These fluctuations are shown more distinctly by the spikes in the permeate flow rate in FIG. 37. These spikes represent the brief release of gas by the system as measured by an instantaneous mass flow meter. Retentate flow rate measurements are taken as a longer-duration time average using a dry test meter. Therefore, the retentate flow rate does not show the instantaneous flow spikes.

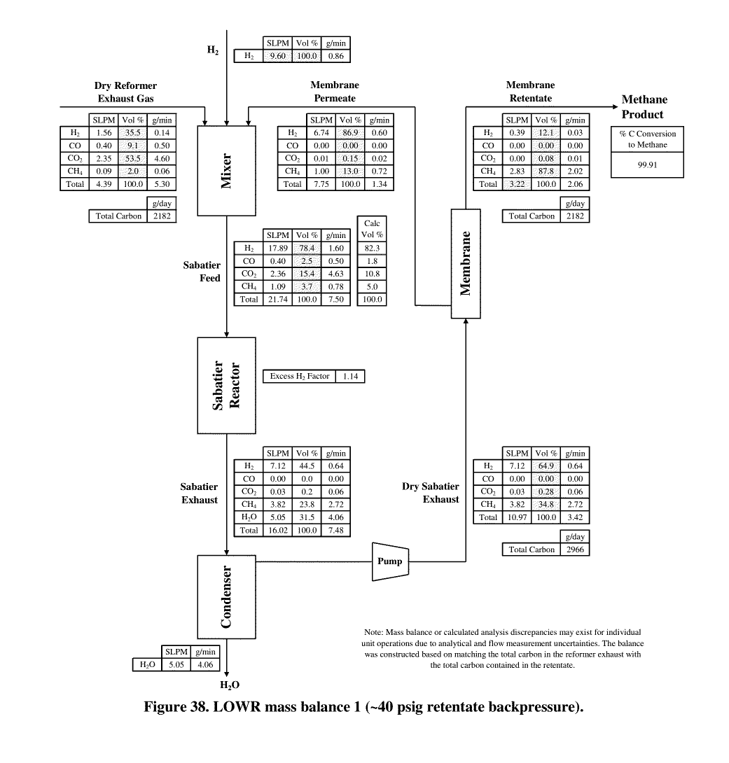

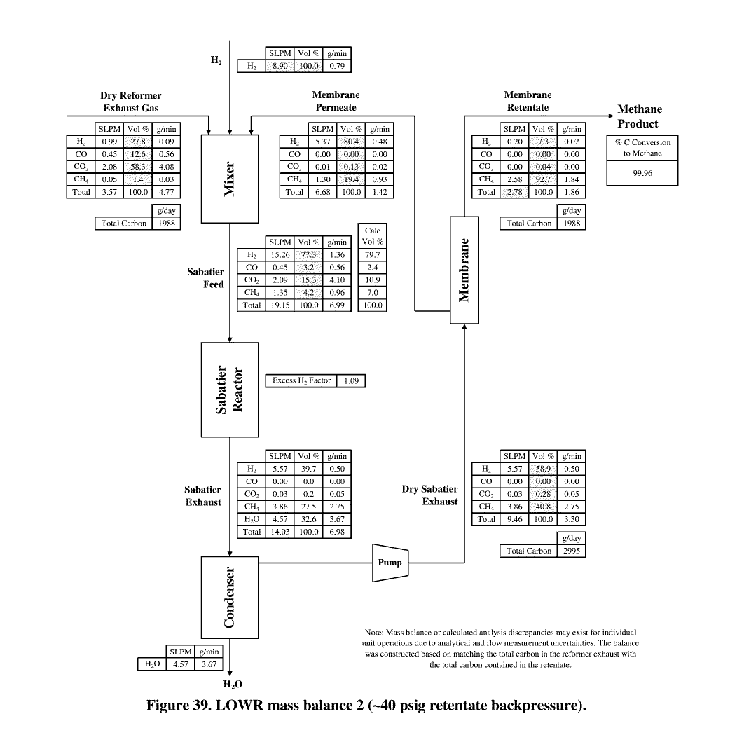

The hydrogen flow rate to the Sabatier reactor was gradually reduced based on the methane product gas analysis and system pressures during the course of the run. Despite the upsets noted above, conversion to methane remained at very high levels. Two steady mass balance periods were selected during the integrated run to collect gas analysis and flow information. Results of these are shown in FIG. 38 and FIG. 39.

Conversions of 99.91 and 99.96 percent of carbon oxides to methane at carbon throughputs of 2.2 and 2.0 kg per day were obtained during mass balance periods 1 and 2, respectively. No carbon monoxide was detected in the methane product in either case, and less than 0.1 volume percent CO₂ was present in the methane product, which contained 88 to 93 percent CH₄. The amount of hydrogen excess in the Sabatier reactor was about 1.1 times the stoichiometric requirement during both mass balance periods. Hydrogen contained in the methane product was seven to 12 volume percent, which is only 0.9 to 1.7 weight percent hydrogen. This hydrogen would be separated during methane liquefaction and returned to the Block 2 unit. Results verified the capabilities of the LOWR during fully integrated operations.

### Example 7

Additional experiments were carried out to refine potential control schemes. A potatoes on charcoal feed similar to that described above was used. Observations during pre-heating and just after starting steam and oxygen showed methane concentrations up to about 10 percent, indicating some pyrolysis of the more-volatile constituents of the feed. This is consistent with elevated reformer gas flow rates noted shortly after starting steam and oxygen flow. Much of the feed in the magazine is exposed to elevated temperatures transmitted from the reaction zone, resulting in pyrolysis (and the accompanying higher reformer exhaust flow containing elevated concentrations of methane). As during previous runs, the hydrogen concentration of the reformer exhaust gradually decreased during the course of operations. This also likely indicates that the feed partially pyrolyzes in the feed magazine just above the oxygenated steam injection ports.

A larger feed magazine could be installed to boost operating times and thereby reduce the significance of the observed startup transients. The feed nearest the reaction zone would still be subject to the startup effects described above, but a greater feed mass would allow longer operations that would reach steady-state conditions in the presence of continuous fresh feed. A larger feed mass that moves steadily down the feed magazine would allow steady-state moisture evaporation and pyrolysis of the feed just above the reaction zone. This would then provide a steady flow rate and composition of gases and solid feed through the oxygenated steam zone.

Attempts were made throughout the next run to adjust the supplemental hydrogen flow based on the continuous retentate CH₄ and CO₂ exhaust gas monitor. The values as well as rates of change of methane and carbon dioxide concentrations in the membrane retentate guided adjustments to the supplemental hydrogen flow to the Sabatier reactor. When conditions were steady, even if not exactly on the target operating parameters, samples were taken to characterize the LOWR Block 1 and Block 2 systems.

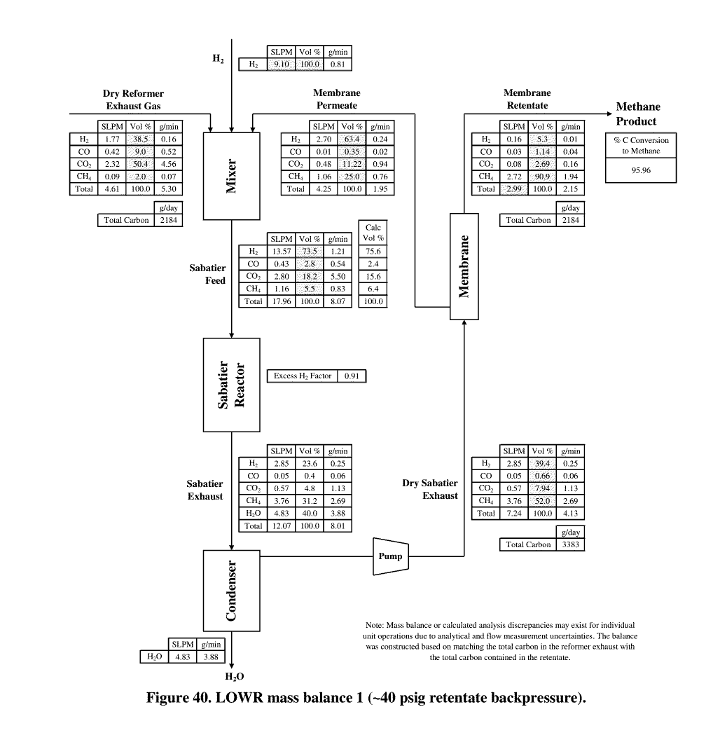

During Material Balance 1 (FIG. 40), there was a deficiency of hydrogen in the Block 2 system as noted by the 0.91 stoichiometric ratio of hydrogen to carbon oxides in the Sabatier reactor inlet. This resulted in the presence of unreacted CO and CO₂ in the membrane retentate. Nevertheless, a carbon conversion of 96 percent was achieved at an average carbon throughput of about 2.2 kg per day.

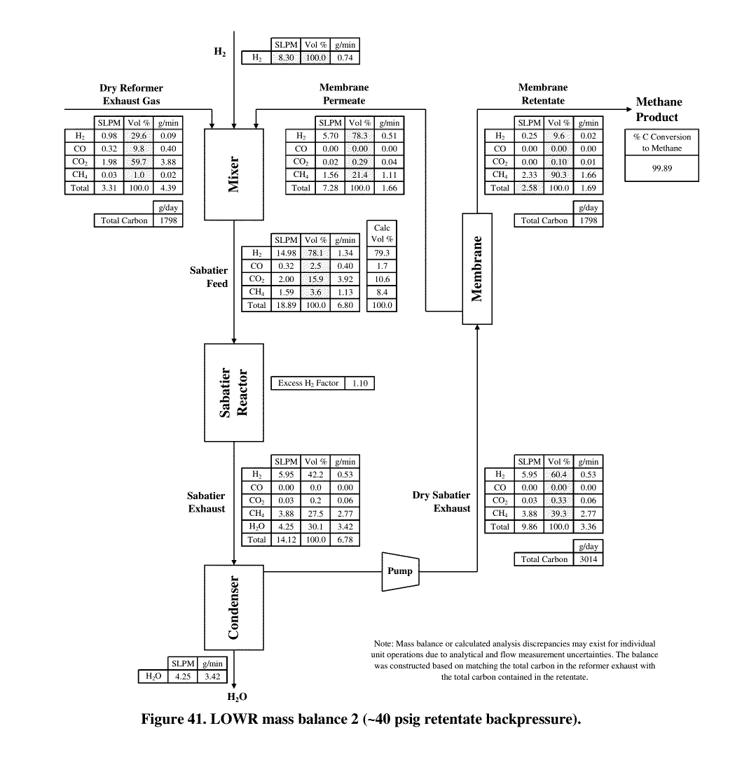

During Material Balance 2 (FIG. 41), the hydrogen was 1.1 times the stoichiometric requirement in the Sabatier reactor feed. Under this condition, a conversion of 99.9 percent carbon to methane was achieved at an average daily carbon throughput of about 1.8 kg.

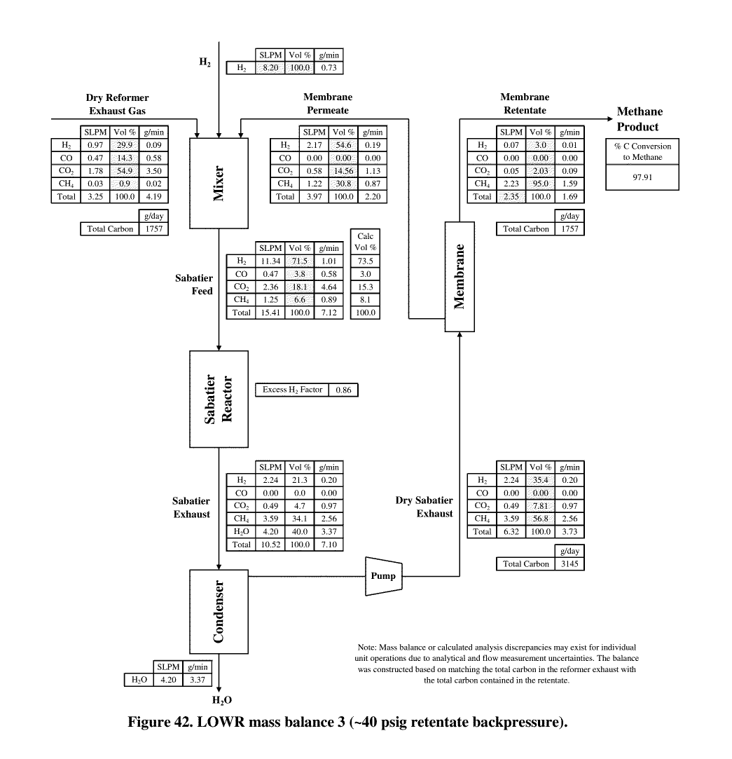

The hydrogen flow was again somewhat deficient during Material Balance 3 (at 0.86 times the stoichiometric requirement) as shown in FIG. 42. Again, this resulted in some unreacted CO₂ reporting to the methane product. However, an overall carbon conversion to methane of 97.9 percent was still achieved.

The results showed that the LOWR Block 2 Sabatier system was very robust to changes in operating conditions and was able to achieve over 95 percent carbon conversion even when manually controlling the supplemental hydrogen flow while tracking a somewhat variable reformer exhaust flow and composition.

### Example 8

Additional LOWR operations centered on processing a NASA High Fidelity Waste Simulant (HFWS). The simulant was prepared to represent wastes typical of a human exploration outpost including food packaging, urine brine, t-shirts, fecal simulant, food, hand/face wipes, towels, health care consumables including shampoo and toothpaste, nitrile gloves, paper, maximum absorbency garments, disinfecting wipes, and tape. A processing rate of 5.4 kilograms per day was specified on the basis of a crew of four producing 1.35 kilograms of waste per crew member per day. The carbon, hydrogen, and oxygen concentrations in the High Fidelity Waste Simulant were then expressed as weight percentage of the dry feed. The following summarizes the analysis used for the LOWR model.

NASA High Fidelity Waste Simulant Composition.

| | | | Mass % |
|---|---|---|---|
| | Mass, kg | Mass % | (dry, |
| Waste | (average | (contaminant- | contaminant- |
| Component | daily rate) | free basis) | free basis) |
| Moisture | 2.397 | 49.0 | — |
| Total Organic Carbon | 1.674 | 34.2 | 67.1 |
| Total Organic | 0.227 | 4.5 | 9.1 |
| Hydrogen | | | |
| Total Organic Oxygen | 0.594 | 12.1 | 23.8 |
| Gaseous Contaminants | 0.254 | — | — |
| Solid Contaminants | 0.254 | — | — |
| Total | 5.400 | 100.0 | 100.0 |

No hardware changes were required to accommodate the High Fidelity Waste Simulant except for chopping the very largest pieces to fit in the reformer feed magazine.

The first run using the High Fidelity Waste Simulant (HFWS) was carried out in a fully integrated mode using 263 grams of HFWS. The feed was loaded on top of 363 grams of charcoal into the empty reformer. An additional layer of carbon (about 25 grams) was added above the HFWS to both help absorb any condensed pyrolysis products evolved from the feed and to ensure a fuel supply in the event that the HFWS was entirely consumed.

The oxygen feed rate was reduced from the previous target of 1.7 SLPM to 1.2 SLPM to allow initial operations at a lower rate. No particular operating difficulties were encountered. However, efforts continued to improve the manual adjustments of supplemental hydrogen flow rate to the Sabatier system based on the exhaust gas methane and carbon dioxide analyzers.

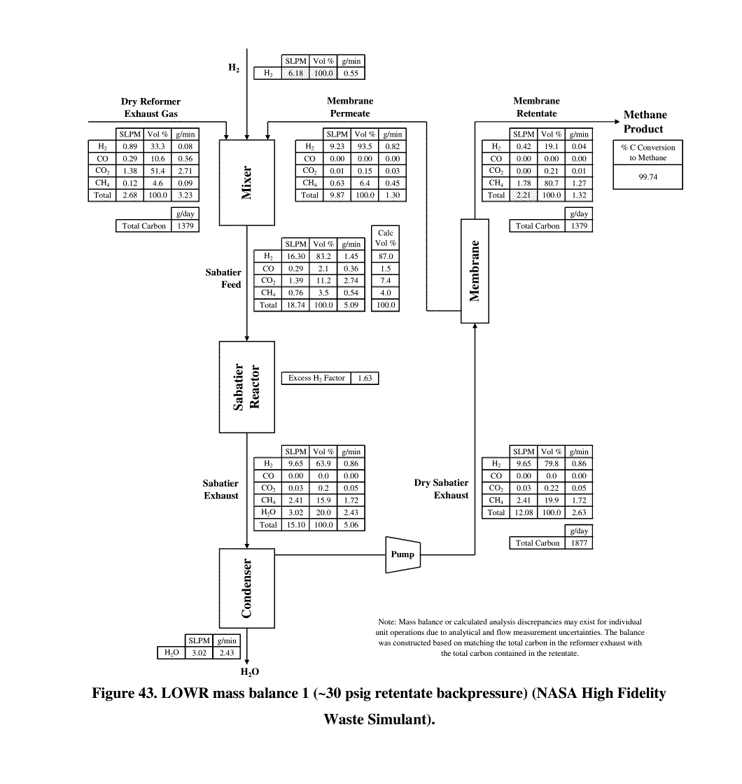

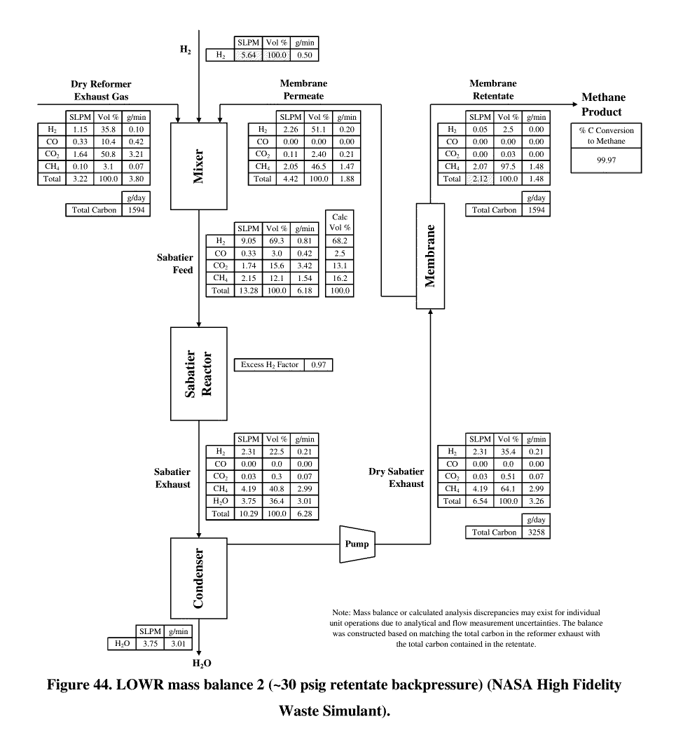

At the target reformer steam and oxygen feed rates of 3.9 and 1.2 SLPM, respectively, carbon oxide conversions of 99.7 to 99.9 percent to methane were achieved at rates of 1.4 to 1.6 kg carbon per day while the HFWS was being fed. Gas samples and flow rates were obtained during two material balance periods in which the HFWS was being fed through the oxygenated steam zone. FIG. 43 and FIG. 44 show results during the two material balance periods.

The Sabatier gas recycle system was operated at a retentate (methane product) pressure of about 30 psig rather than the 40 psig target employed for previous experiments. This allowed for some additional margin when supplemental hydrogen flow was in excess of requirements and system pressures increased. The reduced pressure of the membrane retentate had no significant effect on the overall LOWR performance

Sulfur gas analyses were obtained from the reformer during the run with NASA High Fidelity Waste Simulant. H₂S concentrations of about 40 to 70 ppm were obtained in the dry reformer exhaust gas. COS concentrations ranged from zero to four ppm in the dry reformer exhaust gas. No sulfur compounds were detected downstream of the ZnO sulfur trap.

The LOWR was operated again in integrated mode under conditions similar to those described above. Limited samples were taken, but the reformer and Sabatier profiles were similar to those noted above. A sample of dry reformer exhaust just upstream of the ZnO sulfur trap showed 63 and 1.5 ppm of H₂S and COS, respectively.

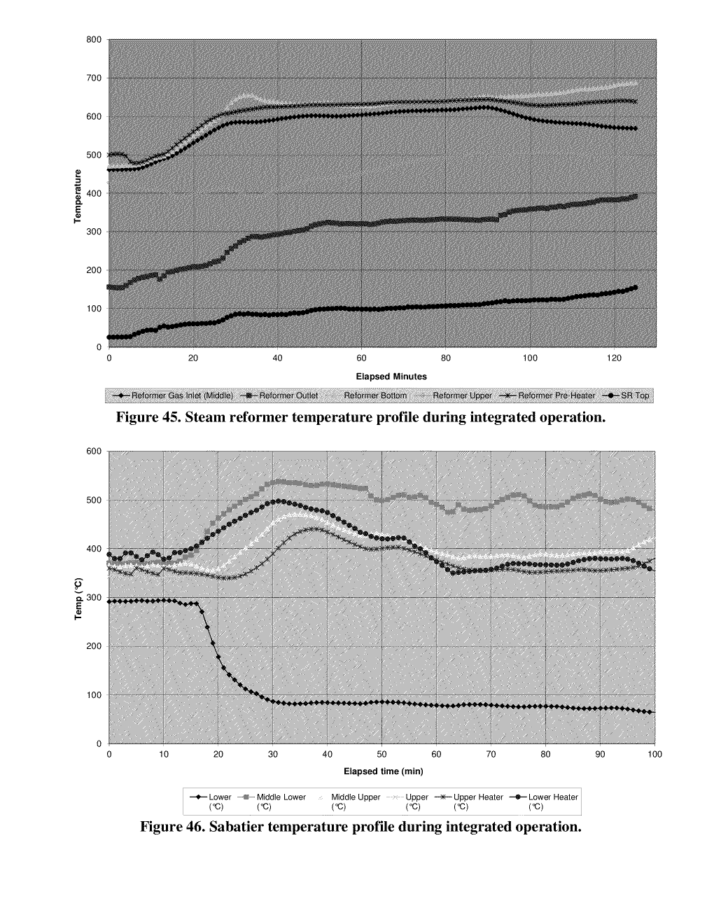

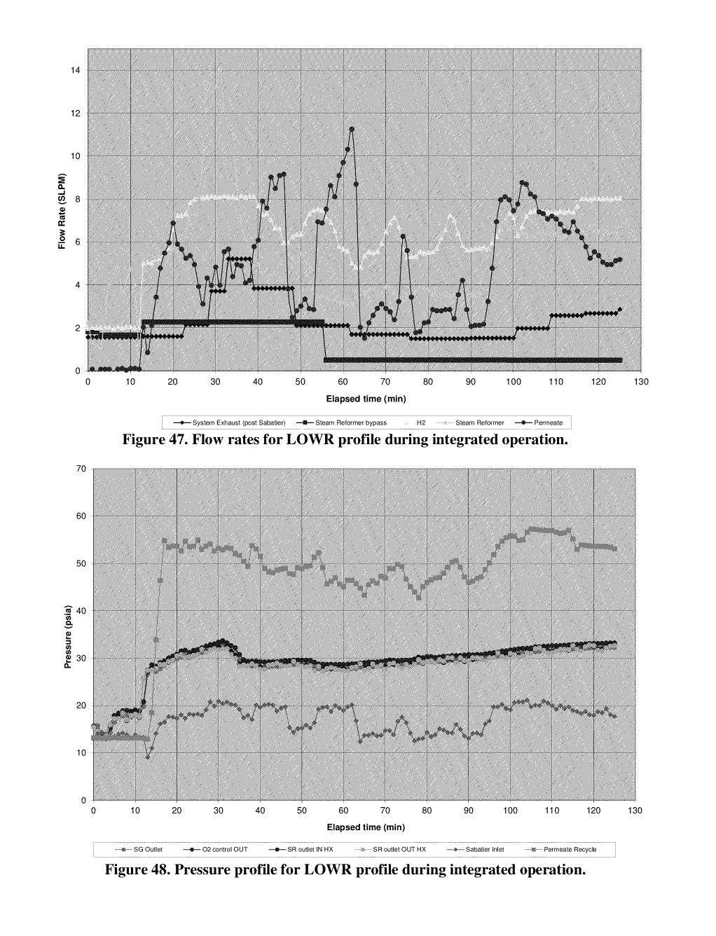

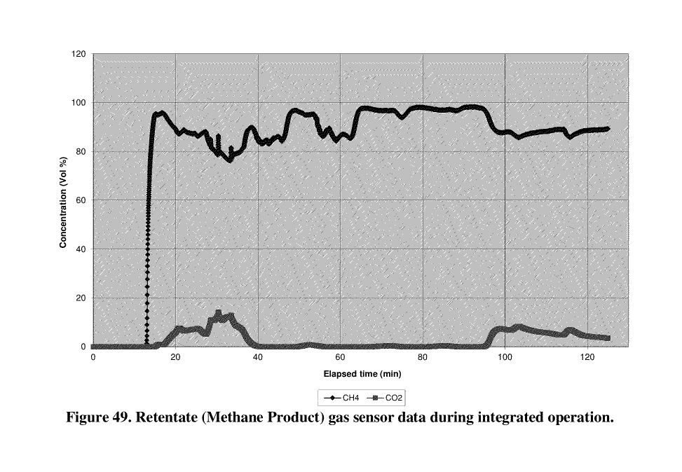

The oxygen flow rate was increased from 1.2 SLPM to 1.7 SLPM for the next run using NASA HFWS feed. This was done to increase the overall process rate and to reduce the fraction of reformer heat lost to the surroundings. The performance of the LOWR during this run was similar to that observed during previous experiments. Temperature, flow, pressure, and methane product analysis profiles are shown in FIG. 45, FIG. 46, FIG. 47, FIG. 48, and FIG. 49.

### Example 9

A distinct surge in reformer exhaust gas flow (as well as methane product flow) was noted during the next experiment. This again was attributed to release of more-volatile feed constituents via pyrolysis of the HFWS feed in the magazine just above the oxygenated steam injection zone plus reforming with the metered rates of oxygen and steam. A dry reformer exhaust analysis during the period of peak flow showed about 43 percent H₂, seven percent CH₄, 14 percent CO, and 35 percent CO₂. The typical flow surge observed during reforming experiments presented some challenge to control supplemental hydrogen flow. However, the membrane retentate CH₄/CO₂ analysis feedback method discussed earlier was used to guide changes in the supplemental H₂ flow. Although an elevated concentration of hydrogen was noted during the flow surge (which would normally reduce supplemental hydrogen flow requirements), the increased overall flow resulted in a need for a higher volumetric flow of supplemental hydrogen.

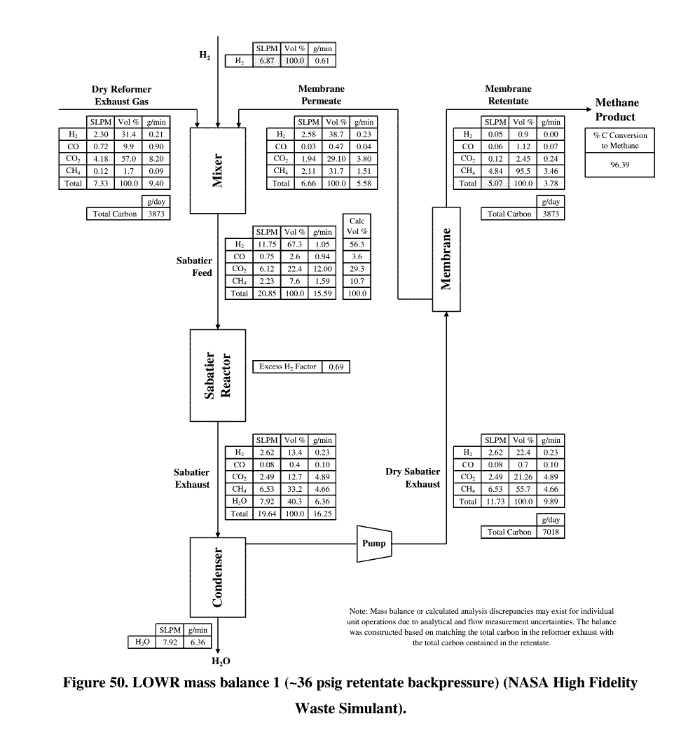

Samples and flow rates were obtained during a reasonably steady material balance period as the flow surge was tailing off. FIG. 50 shows the results. The supplemental hydrogen flow was below target, resulting in a calculated stoichiometric hydrogen ratio of only about 0.7 (versus the target 1.1). As a result, conversion of carbon oxides to methane during this time period was lower, at about 96.4 percent. Although there are some analytical and flow measurement uncertainties, the results again show that the LOWR Block 2 Sabatier system with its integrated membrane separation and recycle is capable of achieving high conversions even under off-nominal conditions. The carbon conversion rate during this test period was about 3.9 kg per day, which was determined to be near the upper limit of the existing Sabatier reactor system, but well in excess of the projected NASA requirements for a crew of four.

The reformer oxygen feed rate was reduced somewhat (from a target of 1.7 SLPM to about 1.6 SLPM) after the initial mass balance period to slow down the overall reforming rate. Gas chromatograph analyses during a second material balance period were corrupted, which made a material balance calculation impossible. Reformer exhaust gas sulfur analyses during the run ranged from 17 to 134 ppm H₂S and about one to three ppm COS. Analyses downstream of the ZnO trap showed zero ppm H₂S and about two ppm COS, indicated that after continued use, the ZnO trap still effectively removes H₂S but allows COS to pass through.

The membrane retentate analysis feedback method discussed above was effective when modest changes to H₂ flow were required. However, the slight delay in applying downstream analysis data to the upstream process flow resulted in some over- or under-correction when larger changes were called for. During the previous run, it was observed that additional measures could be applied for automated control of supplemental hydrogen flow. For example, a mass flow meter installed on the dry reformer exhaust gave immediate indication of the flow change from the reformer. This value, along with the existing CH₄/CO₂ sensor system on the membrane retentate could be used to apply more precise adjustments to the supplemental hydrogen flow rate. The addition of a CH₄/CO₂ sensor system (or better, a CH₄/CO₂/CO sensor system) on the dry reformer exhaust along with the flow data would provide the required feed-forward controls to maintain a very precise addition of supplemental hydrogen. These refinements can be made as the LOWR process development is advanced.

### Example 10

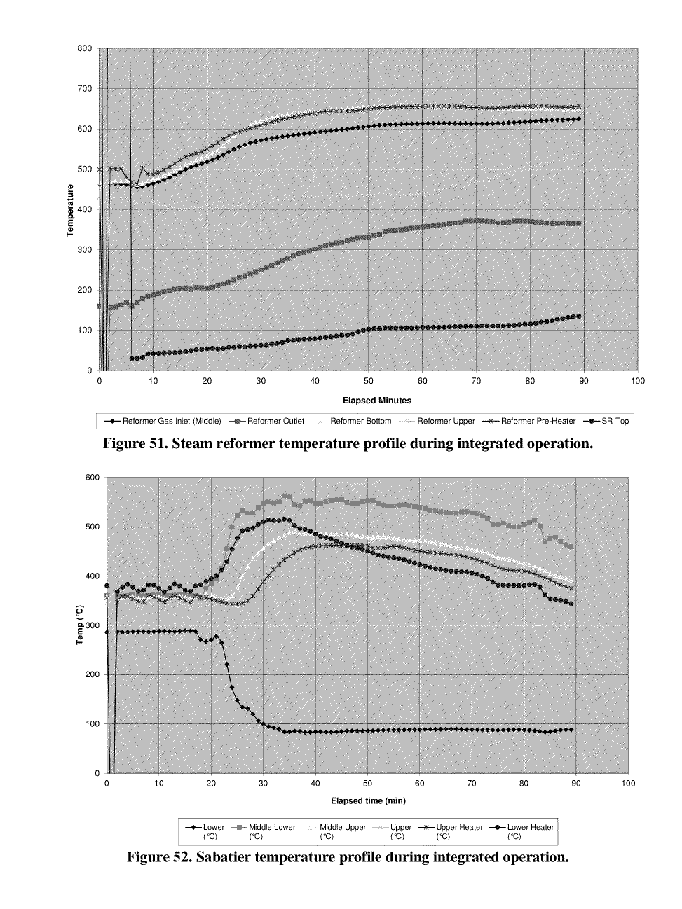

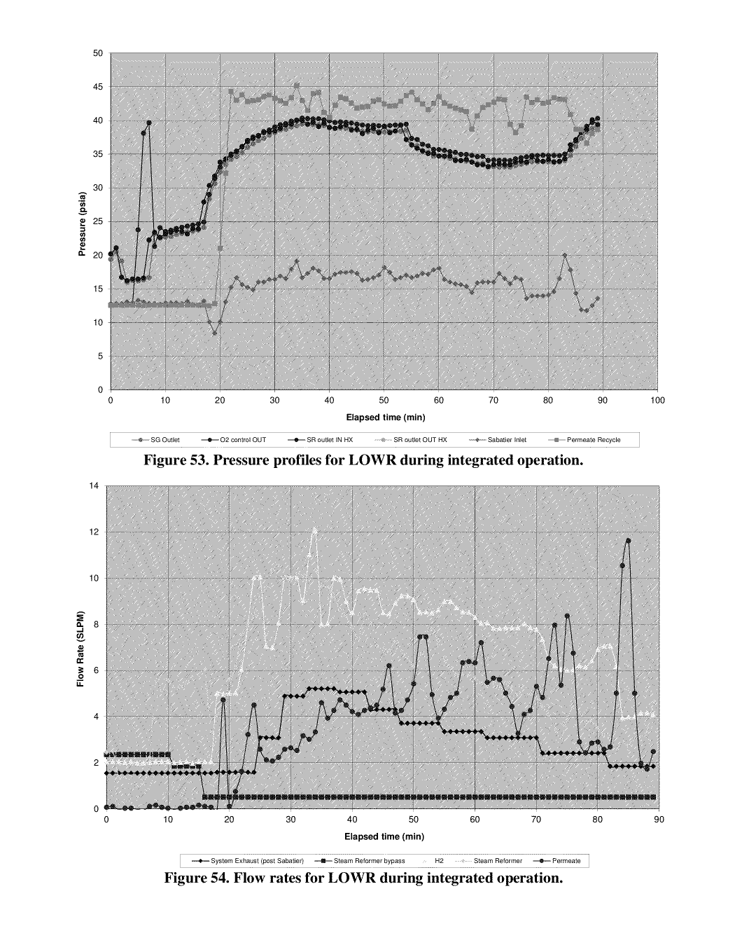

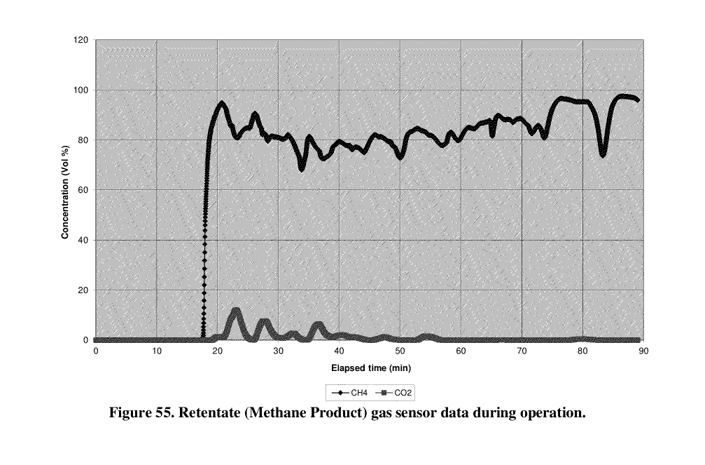

An additional run was made using the NASA High Fidelity Waste Simulant. The objective was to apply knowledge learned so far to LOWR operations under conditions close to the target required process rate. The temperature, flow, pressure, and product gas analysis profiles are shown in FIG. 51, FIG. 52, FIG. 53, FIG. 54 and FIG. 55.

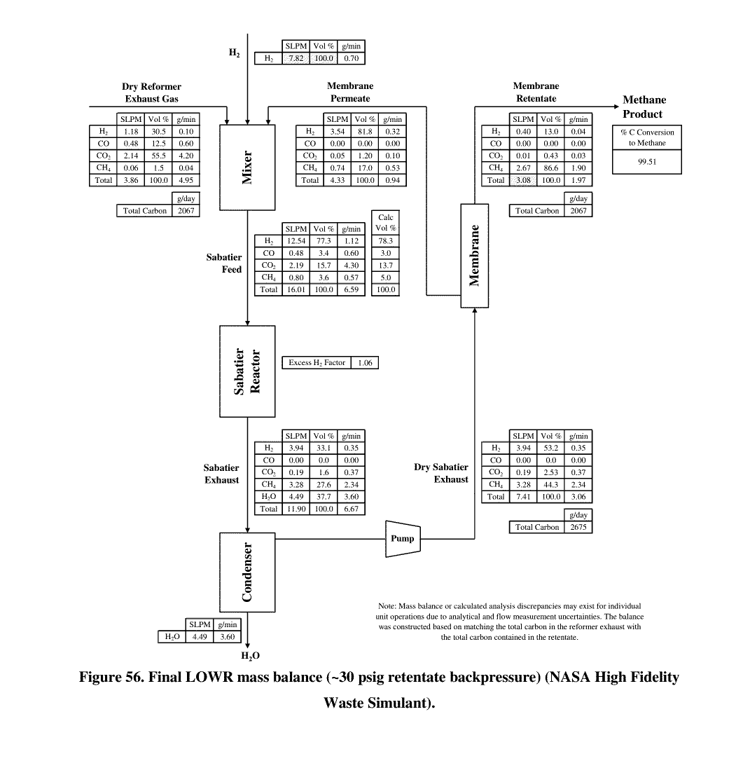

The material balance for the final experiment is shown in FIG. 56. The supplemental hydrogen flow was close to its target based on the calculated 1.06 stoichiometric excess measured at the Sabatier reactor inlet. The conversion of carbon oxides to methane during this period was 99.5 percent, with 0.43 percent CO₂ and no CO reporting to the methane product.

The instantaneous carbon processing rate during the material balance period was about 2.1 kg per day, or 2.7 SLPM CH₄. This compares to the required instantaneous process rate of about 3.3 SLPM CH₄ required for the baseline crew of four operating with waste having the same composition as the High Fidelity Waste Simulant at an on-stream factor of 67 percent. The operating results show that the LOWR can operate successfully at rates representative of full-scale needs.

### Example 11

A semi-empirical model was prepared to incorporate observed experimental results with a material balance for a lunar space outpost example. The model output is presented in a summary block-flow diagram to provide additional clarity as shown in FIG. 57. The summary shows that for the base-line NASA case for a crew of four and waste representative of the High Fidelity Waste Simulant, methane and oxygen product rates of 2.2 and 3.3 kg per day, respectively, would be obtained. Primary LOWR inputs consist of make-up water (at about 0.6 kg per day) and electrical operating power (mostly electrolysis at about 1.6 kWe).

### Example 12

The oxygenated steam reforming procedures described in previous examples are applicable to a process in which the reforming gases are fed to methanation without supplemental hydrogen, as illustrated in FIG. 1. In this example, the reforming gases are fed to the methanation reactor either directly (without removing moisture) or after removing a part or all of the moisture. The advantage of leaving the water present in the reforming gas is that an intermediate condenser is not required, and the water present in the reforming gas will minimize or eliminate carbon formation during methanation when supplemental hydrogen is not added.

In this example, an agricultural waste containing about ten percent moisture and 47 percent carbon, five percent hydrogen, and 38 percent oxygen (all by weight) is subjected to oxygenated steam reformation and subsequent methanation. The approximate composition for such a feed would be C₃₄H₄₆O₂₀. During reforming, ratios of approximately 2:1 H₂O:C and 0.5:1 O₂:C (by mole) are used, and the resulting gas mixture contains an approximate 2:1 ratio of CO₂:CO (by mole). Moisture and oxygen present in the organic matter reduce the amounts of added oxygen and steam. The reforming reaction for this case is shown below. Values were rounded for clarity.

68 H₂O+C₃₄H₄₆O₂₀+7O₂=>46H₂+23CO₂+12CO+45 H₂O

Excess water is used to drive the conversion of the organic matter to gaseous products and to control reaction temperature. Water present in the reformer gas exhaust is left in the methanation feed gas to reduce the possibility of carbon formation. During methanation, carbon monoxide is first converted to methane using available hydrogen in the reforming gas and the balance of available hydrogen is then consumed to convert carbon dioxide to methane.

Thermodynamic equilibrium indicates small amounts of unreacted hydrogen and carbon dioxide would be present after a single pass through a methanation reactor under these conditions. However, the use of a gas separation membrane and recycle of hydrogen- and carbon dioxide-rich gas to the methanation reactor (such as depicted in FIG. 1) would result in virtually complete conversion of hydrogen. The methanation reaction for the reforming gas product is shown below. Values were rounded for clarity.

46H₂+23CO₂+12CO+45H₂O=>15CH₄+20CO₂+63H₂O

Methanation takes place at temperatures between about 300 and 600° C. and pressures between about one and 100 bar.

Results of the example show that after removing water from the methanation product gas, methane-rich and carbon dioxide-rich product gases in the proportions shown above would be obtained when following the general flow sheet shown in FIG. 1.

### Example 13

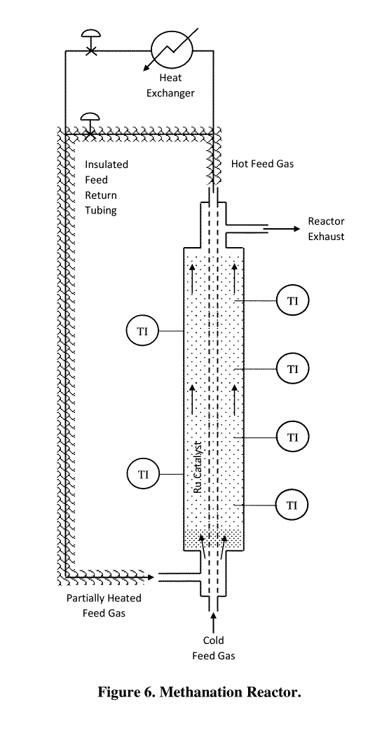

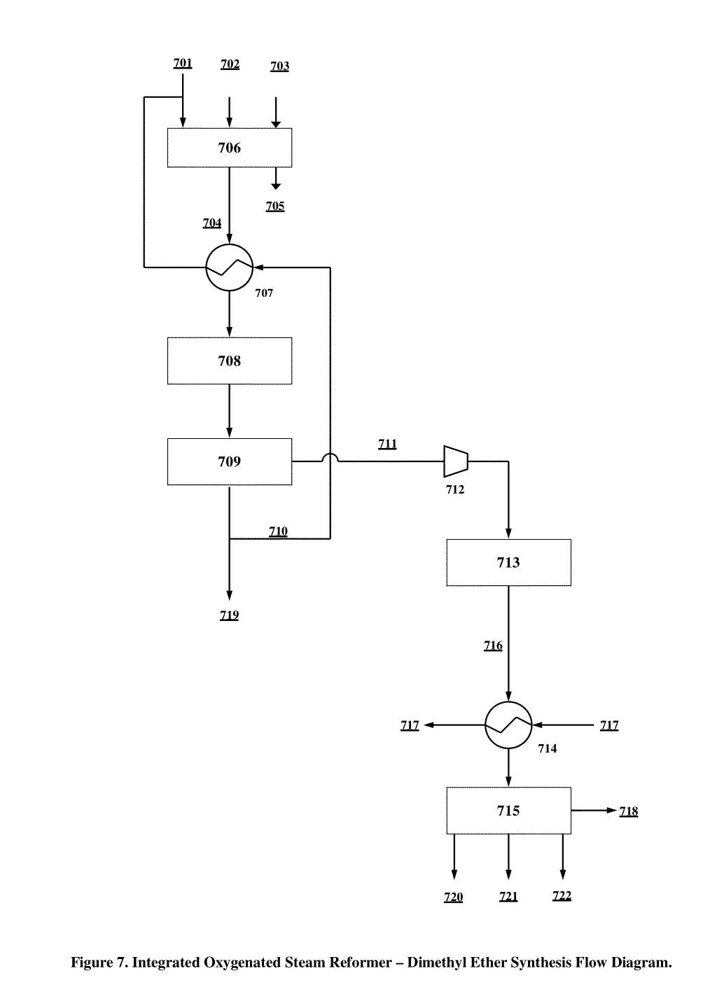

The oxygenated steam reforming procedures described in previous examples are applicable to a process in which the reforming gases are fed to a combined methanol-dimethyl ether synthesis reactor system as illustrated in FIG. 7 to produce significantly higher commercial value from reformer exhaust gases. In this example, the reforming gases are fed to the combined methanol-dimethyl ether reactor after removing moisture.

The dry reforming gases are compressed to a pressure preferably greater than five bar and more preferably to a pressure of at least ten bar and up to 100 bar.

The combined methanol-dimethyl ether synthesis reactor is filled with a mixture of copper-zinc oxide or other catalysts known for conversion of hydrogen and carbon monoxide to methanol and gamma alumina or other catalysts known for conversion of methanol to dimethyl ether. The benefits of combined methanol-dimethyl ether reactions are detailed in the Background section of this patent for generation of the desired dimethyl ether product.

The heat release from simultaneous methanol and dimethyl ether synthesis is about one-half of that per unit of carbon converted in the methanation reactions described above. Heat released from the exothermic methanol and dimethyl ether synthesis reactions is diluted by the presence of unreacted reformer gases including carbon dioxide as well as a portion of the hydrogen and carbon monoxide. A methanol and dimethyl ether synthesis reactor of the general design illustrated in Example 2 would be suitable for the application. Alternatively, a conventional fixed-bed reactor with appropriate thermal controls is also applicable.

A dry reforming gas produced from agricultural waste material of the composition noted in Example 12 would contain about 30 percent CO₂, 15 percent CO, and 55 percent H₂ (on a molar basis). Feeds to oxygenated steam reforming containing different compositions and produced under different reforming conditions vary from these values. In any case, the large majority of carbon monoxide is converted to the desired dimethyl ether and methanol products while the large majority of carbon dioxide remains unreacted.

Thermodynamic analyses of the conversion of the dry gases generated from reforming of agricultural waste to dimethyl ether (DME) and methanol are illustrated in the following table over a range of temperatures and pressures. Further improvement to yield can be made at lower operating temperature (constrained by the minimum temperature required to achieve useful reaction rates) and higher operating pressure (constrained by hardware costs).

Percentage Conversion of Carbon from Dry Reforming
Gas to Dimethyl Ether and Methanol

| | Yield, % of all carbon in feed gas reporting to products | | | | | |
|---|---|---|---|---|---|---|
| Temperature, | 10 bar pressure | | 20 bar pressure | | 30 bar pressure | |
| ° C. | DME | Methanol | DME | Methanol | DME | Methanol |
| 210 | 21.4 | 1.5 | 25.8 | 2.2 | 30.1 | 3.1 |
| 230 | 16.8 | 1.3 | 22.6 | 2.1 | 27.4 | 2.9 |
| 250 | 10.5 | 1.1 | 18.4 | 1.9 | 24.3 | 2.8 |

After removal of the valuable dimethyl ether and methanol products, remaining gases such including carbon dioxide, carbon monoxide, and hydrogen can be further used as fuel or as feedstock to recover additional products.

### Example 14

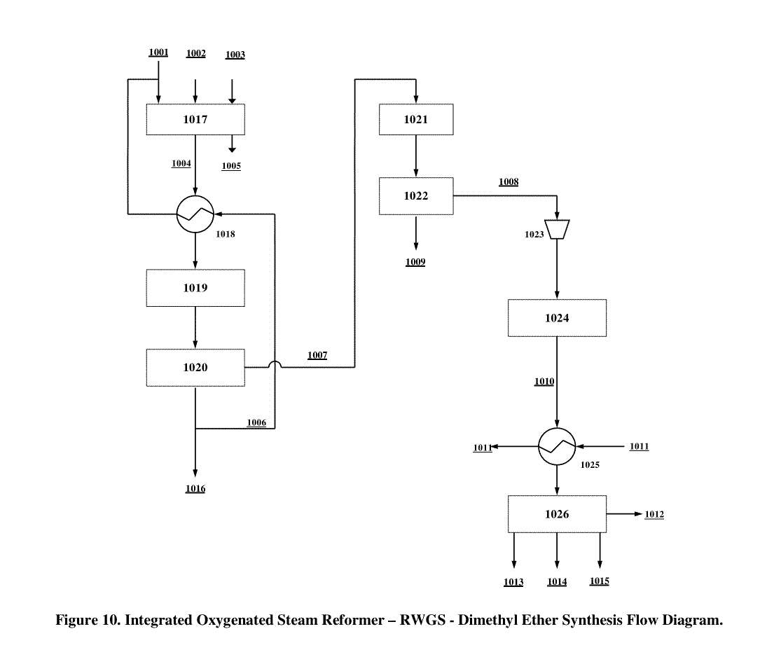

In this example, the same agricultural waste is subjected to oxygenated steam reformation as described in previous examples and the resulting dry product gases are fed to a combined methanol-dimethyl ether synthesis process as illustrated in FIG. 10. The procedures as described in Example 13 are followed in this example with the exception that a reverse water gas shift (RWGS) reaction is performed on the dry reformer gas before feeding to the methanol-dimethyl ether synthesis reactor.

The inclusion of the RWGS step (with subsequent removal of water formed) results in a feed gas to methanol-dimethyl ether synthesis that allows for greater utilization of carbon oxides and hydrogen.

The reverse water gas shift reaction can be carried out over a copper-zinc oxide or other suitable catalyst at a temperature between 300 and 500° C. and pressures of one to 20 bar.

The dry reformer gas composition of 30 moles CO₂, 15 moles CO, and 55 moles H₂ is shifted to a composition of approximately 22 moles CO₂, 23 moles CO, and 46 moles H₂. Water is condensed and removed to produce a dry feed gas to methanol-dimethyl ether synthesis. This feed composition to the combined methanol-dimethyl ether synthesis reactor results in significantly improved yield of the desired dimethyl ether product while slightly decreasing the yield of methanol. The following table compares the effect of including an RWGS step upstream of the methanol-dimethyl ether synthesis step.

Percentage Conversion of Carbon from Dry Reforming Gas to
Dimethyl Ether and Methanol At 20 bar pressure and 230° C.

| | Yield, % of all carbon in feed gas reporting to products | | | |
|---|---|---|---|---|
| Temperature, | No RWGS | | With RWGS | |
| ° C. | DME | Methanol | DME | Methanol |
| 230 | 22.6 | 2.1 | 31.5 | 1.7 |

Improvements to dimethyl ether yield are obtained at all of the conditions cited above in Example 13.

### Example 15

Water condensed from the reformer exhaust is typically brown or amber in color and has a distinct pyrolysis odor regardless of the feed type. The reformer condensate represents the excess steam used to help drive the reforming reactions to completion. A large majority of the water condensed from the reformer exhaust gas is typically recycled to the reformer. This portion of the reformer condensate would require little or no treatment to remove organic matter due to its exposure to high temperature steam and oxygen up re-introduction to the reformer. Experiments showed that filtration through cellulose or paper elements prior to recycle removes sticky tars that could foul heat exchanger surfaces. After use, the light-weight filter elements could be fed to the reformer to recover trapped tars and filter media as methane and oxygen products. Reformer condensate after filtration still contains dissolved organic matter. The conventional activated carbon and deionizing resin used for electrolysis water pre-treatment would likely be effective for its removal. However, the activated carbon would saturate faster than would otherwise be the case, and alternate reformer condensate treatments are desirable.

The amount of organic contamination in the condensate appeared to be greatest during reformer start up. This was probably due to pre-heating of the reformer, which led to pyrolysis of the more-volatile waste constituents just prior to initiating steam and oxygen flows. Consequently, the amount of organic matter reporting to the reformer condensate could be reduced with longer duration reformer operations to minimize startup transients.

A composite condensate sample was analyzed as a starting point in designing a condensate clean up system. The condensate appeared to be an amber tinted emulsion with some floating and suspended char fines together with agglomerated viscous tar globules adhering to the sides of a plastic storage container. It possessed a distinct pyrolysis tar odor. A simple filtration experiment was conducted using conventional coffee filters first by decanting some of the supernatant liquid off the top of the long settled mixture and after vigorously shaking the container in an attempt to recombine the constituents. The filtrate from the decanted liquid appeared to show a slight floating oil layer. After about two hours of resting undisturbed, tar-like suspended particles dropped to the bottom of the bowl containing the pre-mixed condensate sample. Filtrate from the decanted sample remained nearly homogenous. pH was between eight and nine. Even after filtration, the reformer condensates had an amber to brown color and an odor indicating the presence of organic matter.

Based on the characteristics of the reformer condensate, a water treatment method to accommodate requirements of reformer water recycle and electrolysis was sought with the objective of using a minimum of consumables, hardware, and power. One method of dealing with reformer condensate organic contaminants is to minimize their formation. Olivine has been reported to be effective for prevention of tar formation when reacted with reformer gas at temperatures above 800° C. Note that olivine is a mineral that occurs in lunar regolith and could potentially be concentrated and recovered as a resource for an aerospace application of the present invention. Similar methods can be employed on Earth.

A reforming experiment was carried out with olivine blended into the steam reformer feed to determine the effects on reforming and on tar formation. The olivine was blended with the feed to provide exposure to maximum tar destruction temperatures (in the oxygenated steam injection zone). A one-inch (2.54 cm) layer of granular olivine was first placed above the charcoal bed prior to startup. Potatoes containing a 20% by weight addition of olivine were then fed to the reformer magazine. Inclusion of the olivine did not alter the reforming characteristics of the experiment with respect to temperature and pressure profiles and gas analysis results. Results were similar to previous potato reforming experiments performed without olivine addition.

Condensate from the experiment run with olivine in the feed and two others similar to it without olivine addition were analyzed by gas chromatography/mass spectrometry (GC/MS) to characterize the nature of the contaminants and to establish effects of olivine on tar formation and water quality. Each condensate was filtered (0.45 micron) to remove particulates. The filters plugged, indicating the presence of fine particles and possibly polymerized organic matter in the raw condensates. Additional filtration experiments were conducted using ordinary coffee filters, which were effective for removal of coarser, polymerized tars. Filtration generally trapped dark organic matter and resulted in a lighter color filtrate.

Semi-quantitative GC/MS analyses were obtained on reformer condensate composite samples with and without olivine addition to the reformer feed. For this series of analyses, the GC/MS response from filtered reformer condensates was correlated to typical responses for other calibrated organic compounds. Analysis of filtered reformer condensates showed primarily phenols including phenol, 2-methyl phenol, 3-methyl phenol, 2,4-di-methyl phenol, 3,5-di-methyl phenol, and 3,4-di-methyl phenol. Phenol is generally at the highest concentration (typically 100 to 200 ppm in the filtered condensates) with others in decreasing concentrations down to the 10 ppm range in the order listed above. Small amounts (ten ppm range) of ethanediol and creosol were also detected. Many other much smaller peaks were noted in the GC/MS results, but were not tracked for this experiment.

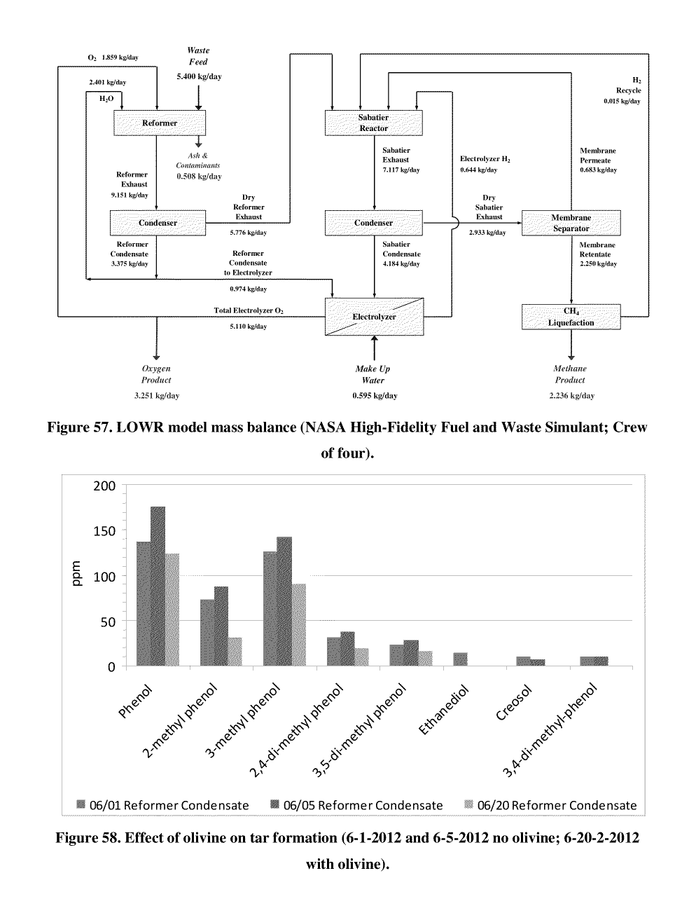

In all cases, condensate from the experiment in which olivine was added showed lower concentrations of contaminants. 3,4-di-methyl phenol, ethanediol, and creosol present in experiments without olivine were undetected in the condensate generated during the olivine experiment. FIG. 58 summarizes the concentrations of contaminants in reformer condensates produced with and without olivine in the reformer. These scouting tests indicated potential for reducing tar formation by olivine addition, but conditions were not optimized, and further treatment would likely be required for the portion of condensate directed to electrolysis.

Distillation of the reformer condensate was investigated as a potential means of separating contaminants from water. For this experiment, about 200 ml of condensate was loaded into a stainless steel distillation container venting to a condenser. The contents were heated initially to about 80° C. After collecting a small amount of condensate (about six ml), the distillation container was further heated to boil at a low rate while collecting distillate. The “low heat distillate” was collected, and the heat rate was increased to a faster boil. A “high heat distillate” was then collected, and the distillation container was cooled. The distillation bottoms, low heat distillate, and high heat distillate were recovered in roughly equal proportions. The feed, distillates, and distillation bottoms were analyzed by GC/MS as summarized in FIG. 59.

The distillates were clear while the distillation bottoms were a darker brown/amber color than the starting reformer condensate. Phenol compounds were concentrated in distillates (and were mostly removed from the distillation bottoms). The results appear to indicate that darker, possibly water-insoluble organic matter concentrated in the bottoms while water-soluble phenols reported to distillates. The concentrations of phenols were lower in all samples than in the feed after distillation treatment, indicating possible partial oxidation or destruction of phenols during distillation. Although distillation was effective in separating different organic contaminants from the reformer condensate, additional treatment would be required before using any of the fractions for electrolysis.

Oxidation of the reformer condensate organic matter to CO₂ and H₂O would be desirable to produce clean water while generating byproducts that are completely compatible with the oxygenated steam reforming chemistry. The effectiveness of oxidation for removal of organic matter from reformer condensates was investigated using a composite condensate sample collected during several reformer runs. Two samples were prepared for initial comparative analysis of treatment using hydrogen peroxide. One sample was prepared as a 1:1 mixture of reformer condensate and 35 weight percent hydrogen peroxide. The other sample was prepared as a 1:1 mixture of condensate and water to provide a baseline for comparison. As detailed below, the hydrogen-peroxide treated condensate exhibited a gradual increase in clarity, becoming nearly clear after several days of intermittent agitation. The scouting test result showed the effectiveness of oxidation to remove organic matter from reformer condensate. GC-MS analysis showed significant decreases in the concentrations of organic compounds in the H₂O₂ treated condensate. All compounds detected in the untreated sample were removed or reduced in concentration by treatment with hydrogen peroxide. No new compounds appear to have been formed by the peroxide treatment.

Literature citations show that catalytic wet oxidation with air or oxygen at 150 to 300° C. in the aqueous phase at the saturated steam pressure could be effective. Consequently, experiments were carried out using catalytic oxidation of reformer condensate in an effort to convert organic compounds into CO₂ and H₂O. Initial experiments were carried out using reformer condensate in a sealed stainless steel container using platinum catalyst. The batch wet air oxidation (WAO) bench test rig was built using a 300 cc Hoke® bottle. Initial experiments were conducted using a 100 cc sample of reformer condensate and 10 g catalyst with the head space pre-charged to 100 psig with compressed. Air. The reactor was heated to the point where the pressure reached 370 to 400 psig, or about 180° C. After 40 minutes the reactor was shut down and allowed to cool overnight. GC analysis of the head space gas gave an indication of the breakdown of organic compounds. Liquid samples were prepared using a 0.45 μm syringe filter to protect the column on the GC/MS. The initial scouting experiment showed no significant effect as the treated condensate still had an odor and a brown color. Additional WAO tests were conducted on reformer condensate using palladium catalyst (Acros Organics CAS: 7440-05-3) and compressed air. GC/TCD analysis of the head space gas showed a reduction in oxygen concentration to less than 10% and a small amount (0.08%) of CH₄ and 0.15% CO₂, which indicated at least some conversion. Less-severe oxidation conditions leading to formation of intermediate compounds would explain the relatively small amount of CO₂ produced against the relatively large amount of 02 consumed. The WAO product liquid after processing seemed slightly lighter but still had a distinctive tar odor. A sample was prepared using a 0.45 μm filter to protect the GC/MS, which yielded 19.9 ppm total phenol and some p-cresol, indicating about a 50 percent reduction.

The catalytic oxidation treatment focus shifted to copper-rich catalysts, which are reported to be effective for the application. A high activity, medium temperature shift catalyst (Unicat Catalyst Technologies MTS-401) was tested next. The composition is listed as >42% CuO, 40% ZnO, 2% graphite, and the balance Al₂O₃. After cleaning, the reactor was loaded and run using a distilled water blank to determine effects of the catalyst graphite coating. GC/TCD analysis on the headspace following the short term (40 min), low temperature and pressure (178° C. & 400 psig) distilled water blank test did show very slightly elevated CO₂ levels (0.09%). The liquid phase had a very slight tint that was removed by the 0.45 μm GC/MS filter. No organics were detected in the product liquid vial.

The wet air oxidation test rig was then loaded with reformer condensate and processed with the MTS-401 catalyst and air. Pressure and temperature response was similar (404 psig & 180° C.). The headspace showed very little CO₂ accumulation (0.03%). The WAO condensate was still dark with some foaming evident, giving off a strong effervescent ammonia odor. The filtered product liquid now had a distinctive blue tint, likely the result of leaching copper from the catalyst. GC/MS results showed only 12 ppm total phenol and some p-cresol. Results showed at least partial effectiveness in destroying organic condensate contaminants.

An alternative copper-zeolite catalyst prepared in-house was tested next. In a similar fashion the WAO rig was cleaned out and used to process 100 cc of reformer condensate with air (400 psig & 187 C for 40 min) using 10 g Cu-zeolite catalyst. The product liquid was still dark and odorous but very frothy and virtually clear after filtering (see FIG. 4). This time there was a significant increase in CO₂ to between 0.8 to 0.9% in the head space, and the phenol in the liquid phase dropped to 3.6 ppm from 45.7 ppm in the feed (a 92 percent reduction).

Next, compressed oxygen was used instead of air to try to drive the oxidation reaction more fully by increasing the partial pressure within the head space. In addition, pure oxygen would be more compatible with the oxygenated steam reformer. Therefore, the experiment was repeated with the same Cu-zeolite catalyst and reformer condensate under similar conditions (378 psig 170 C) but with oxygen and a run time of about 6.5 hours.

Following wet oxidation the processed reformer condensate was still dark and gave off a faint ammonia odor. Head gas consisted of 3% CO₂, over triple the concentration of previous analysis, and indication that N₂ is forming (possibly from decomposition of NH₃) with readings of 0.6 to 3.4% N₂ using GC/TCD. The product liquid appeared to retain dissolved gas after depressurization as evident from the bubbles clinging to the sides of the beaker. The liquid sample was completely clear after filtration through a 0.45 μm syringe filter to protect the GC/MS column No organic species were detected. It was also noted that after allowing the collection bottle from the long term Cu-zeolite oxidation experiment to sit undisturbed over the weekend that all the particles settled out of solution. Additional tests showed the pH of product liquid had not changed appreciably but that copper was detected at about three ppm in solution.

Although there was virtually complete conversion of organics by Cu-zeolite treatment, the product liquid still had appreciable particulate matter and a slight odor. It was then decided to create a catalyst from pure copper. This was done by milling shavings from a conventional copper tube. After washing the reactor it was filled with 100 g of copper shavings and the experiment was repeated using 100 cc reformer condensate and a 100 psig oxygen head under similar conditions (400 psig and 180 C) for just over six hours. This time CO₂ from the GC/TCD was the highest yet at about 9 percent, with nitrogen ranging from 0.5 to 0.7 percent, and oxygen correspondingly reduced to about 86 percent. The liquid product was still dark and had a faint coal tar odor. After filtering a light blue tinted product liquid remained. GC/MS analysis shows total phenol dropped from 70 ppm in the feed to non-detectable values. Although WAO experiments described above were at least partially successful in destroying the liquid phase organics, there appeared to be a residual particulate char that remained after oxidation and some remaining liquid phase organics.

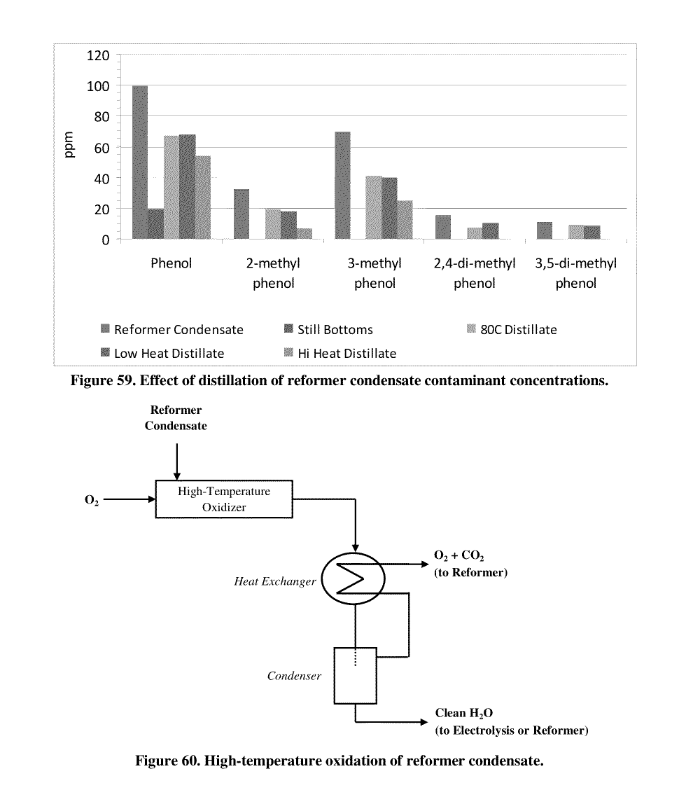

Although WAO experiments described above were at least partially successful in destroying the liquid phase organics, there appeared to be a residual particulate char that remained after oxidation and some remaining liquid phase organics. The following describes further treatments carried out at higher temperatures. A new high-temperature oxygen-steam treatment test rig was developed in a form that could serve as a prototype for the reformer system. The rig was conservatively designed to process all reformer condensate (including the portion recycled to the reformer as well as the smaller portion fed to electrolysis) using oxygen at the rate required for reforming. In practice, such treatment might be applied only to the portion of condensate directed to electrolysis, but testing was conducted at the proportion of total reformer oxygen flow to the entire condensate flow. When installed as part of the reformer, the treatment process would similar to that shown schematically in FIG. 60.

A reactor using low-volume, low-pressure oxygen gas to disperse condensate for improved evaporation of liquid droplets was designed and built. The condensate was metered through a 1/16-inch (1.6-mm) diameter tube concentric within the oxygen feed tube. Oxygen at operating pressures of less than 30 psig produced a conical pattern of fine streaming mist. The wet oxidation steam generator test rig was designed to provide a vapor retention time of 0.5 seconds with a one cc/min reformer condensate flow rate and an initial target steam temperature of 550° C. The main reactor consists of a 12″ long×½″ diameter tube, less the volume of the 6″ long×¼″ diameter internal cartridge heater.

The reformer condensate was fed to the injection nozzle at a constant rate by a syringe pump. In the lab rig, a 24″×⅜″ long un-insulated radiant tube provided additional retention time before the vapor entered a chilled water heat exchanger and knockout accumulator with a condensate drain valve. A gas sample port was installed downstream of the condenser. Particulate matter and larger tar globules were removed from the LOWR reformer condensate prior to treatment. This was done to help prevent plugging of the fine diameter condensate injector. A conventional coffee filter, which typically will retain any particles above 10-15 μm, was used for gravity filtration. In practice, lightweight filter media containing tars could be fed to the reformer to recover the trapped organics and filter media as methane and oxygen products.

Experiments showed that pre-heating the condensate to near boiling substantially improved the filtration rate by apparent dispersion or dissolution of tars. When preheated, the bulk of the reformer condensate passed through the filter almost immediately. Overall, it took over twice as long to filter cold condensate than hot reformer condensate.

During initial high-temperature oxidation experiments, the reactor was preheated to over 200° C. and oxygen flow was started. The reactor shell heat tape and internal cartridge heater were each manually controlled using separate Variac-type autotransformers. The system was started on distilled water while stabilizing flows and temperatures. Initial shakedown tests were set to represent the approximate operating conditions of the LOWR reformer (4.6 cc/min water and 1.2 SLPM O₂). A temperature of 550° C. was selected for initial use. A syringe filled with condensate was then swapped for the distilled water syringe on-the-fly. No significant reaction exotherm was noted, indicating a relatively low concentration of organic matter or limited reaction of the reformer condensate.

Early tests at the initial target temperature of about 550° C. produced dark, odorous condensate occasional foaming and bubbling upon discharge from the condensate drain tube. The exhaust smelled like smoke and the condensate came out black when operated at 550° C. Flow rates were reduced by half again and then back to higher oxygen flow rates at reduced condensate flow, yielding only marginally better visual results. However, the 0.45 micron GC/MS filters retained noticeably fewer particles and the sample filtrate was clearer. Finally, the inside reactor temperature was raised to near the maximum operating temperature of the reactor shell heat tape (750° C.) and the condensate changed from black to amber, the GC/MS filter showed very little particulate matter, and the filtrate sample was perfectly clear. GC/TCD results for this initial scouting run showed 1.5-2% CO₂, 5-10% N₂, 85-91% O₂ and up to 0.7% H₂ in the non-condensable portion of the exhaust gas. This suggested that ammonia was being broken down along with the hydrocarbons. The processed condensate was progressively lower in total phenols, ranging from 96 ppm in the feed to <ten ppm in the high operating temperature sample. It seemed that the color of the treated condensate changed from amber to clear when the reactor was operated with an inside temperature above 600° C. but the vapor wafting from the condenser drain still smelled of smoke. At the lowest liquid injection rate and double the oxygen ratio the amber tint returned. Above 800° C. inside (<400° C. surface) the condensate was clear and there was an apparent ammonia odor.

Prior to the next set of experiments, the test apparatus was operated at high temperatures with oxygen and distilled water to remove any internal deposits that may have formed during initial lower-temperature oxidation experiments. The feed syringe was switched to reformer condensate and the high temperature oxidation condensate remained clear with a very slight amber tint and an apparent ammonia odor. Dipping below 700 C there were a few bubbles spitting out of the drain and the ammonia odor seemed to become stronger. The temperature was ramped up, and fine black particles appeared and the condensate still had a slight tint. Flow rates were successively reduced, and additional samples were taken before switching to distilled water at high flow again to flush out the reactor before shutting down.

The internal temperature of the high-temperature oxidation rig was increased to over 800° C. for the next experiment, which pushed the surface temperature to over 600° C. while feeding distilled water and oxygen. Operations continued until the reactor was thoroughly flushed to remove residues from previous experiments. When reformer condensate was fed while operating at high temperatures, a small (10° C.) bump in inside temperature was noted as the reformer condensate reached the reactor. At this point (845° C. inside/680° C. surface) the vapor had a very faint burnt odor but no ammonia scent, and the CO₂ and N₂ spiked to 3.4% and 14%, respectively. The remaining samples were taken while holding the reactor at about 775° C. inside with 600° C. surface temperatures over the next hour. During this period, the condensate was consistently clear and no particulate was evident in the GC/MS filters.

The reformer condensate used as feed for the test above had 194 ppm total phenols, while the final sample showed only 1.3 ppm phenol and no other organics. Another set of high-temperature, non-catalytic oxidation treatments was carried out after setting flow rates to 0.5 cc/min reformer condensate and 0.24 SLPM O₂ with an internal and external temperatures of about 750° C. The samples were clear with only a slight odor.

After successfully demonstrating the feasibility of cleaning up virtually all the organics in the reformer condensate, activated carbon was investigated as a polishing agent to catch any trace organics that may pass through the high-temperature oxidation treatment. One sample that had a very slight tint from the last lower-flow test and one from the previous higher-flow test that had a very faint ammonia odor were treated overnight with two different types of activated carbon. In very early tests on raw reformer condensate it was believed Aqua Technologies aquarium carbon was slightly more active in reducing the amber tint than the Norit GF45 pelletized activated carbon. Neither carbon was pre-rinsed so there was a considerable amount of fines caught by the GC/MS filters. All samples were clear after treatment. In addition, the ammonia odor was completely gone from higher flow sample and the phenol concentration dropped from 1.3 ppm to non-detect, as well. The low flow samples were later determined to have no detectable organics, both before and after activated carbon treatment.

Finally, the high-temperature oxidation apparatus was operated to demonstrate its performance on reformer condensate gathered from a NASA High Fidelity Waste Simulant during reformer operations. This condensate differs slightly from reformer condensates accumulated from processing charcoal, plastic, dog feces or potatoes in that it appears to have a lighter amber color, is slightly oilier, has less particulate and contains more tar. The coffee filter used to take out coarse particles was visibly cleaner after preparing a composite reformer condensate sample for treatment.

The wet oxidation reactor was started up using distilled water at 0.5 cc/min and oxygen at 0.24 SLPM. Reformer condensate was introduced when the reactor temperature was about 775° C. inside and about 650° C. shell. When the reformer condensate reacted, the reactor temperature increased to as much as 800° C. Treated condensate samples were taken at the initial hottest temperature, again when the temperature was allowed to drop below 700 C, and again when the temperature was steady at 750 C. The non-condensable gas CO₂ concentration was higher, ranging from 3.5 to 5.4%. N₂ was generally about 1.3%. None of the samples emitted a discernible odor. Consistent with the high CO₂ readings, the untreated reformer condensate registered 507 ppm total phenol along with other minor constituents, like catechol, caprolactam and possibly glucopyranose. The 800° C. sample contained 4.1 ppm phenol (probably because the cartridge heater was off for most of the time it was being collected after the reactor temperature shot up to >800° C.). The last two samples showed no detectable organics.

Before the GC/MS results were available for the treated condensate samples, aliquots from the 700 and 750° C. samples were treated using the two different activated carbons. All samples were clear following filtration of the carbon-treated samples and no organics were detected (both before and after treatment). pH values between about 7.5 and 8.5 were recorded on the treated condensates.

Results showed that oxidation of organic compounds in LOWR reformer condensates can be completely removed by an initial filtration step to remove particulate matter and tars followed by non-catalytic, high-temperature oxidation to destroy organic compounds. Results are summarized in the table below for reformer condensates produced from potatoes and charcoal as well as a NASA High Fidelity Waste Simulant. Under the optimum high-temperature oxidation treatment conditions of about 750° C., no organic matter was detected (and activated carbon had no additional benefit).

Effect of High-Temperature Oxidation on Organic Matter in Reformer Condensates
(treatment at 0.5 cc/min condensate and 0.24 SLPM oxygen)

| | | GC/MS Analysis, mg/L | | | | | | |
|---|---|---|---|---|---|---|---|---|
| Reformer | Reformer Condensate | | 2 Methyl | 2 Methoxy | 4 Methyl | 2,4 Dimethyl | 3,4 Dimethyl | Total |
| Feed | Treatment | Phenol | Phenol | Phenol | Phenol | Phenol | Phenol | Phenols |
| Potatoes/ | None (As-Produced) | 79 | 22 | 10 | 58 | 11 | 14 | 194 |
| Charcoal | After Oxidation at ~750° C. | 0 | 0 | 0 | 0 | 0 | 0 | 0 |
| | After Oxidation/Activated C | 0 | 0 | 0 | 0 | 0 | 0 | 0 |
| NASA | None (As-Produced) | 507 | 118 | 41 | 306 | 45 | 49 | 1064 |
| HFWS | After Oxidation at ~750° C. | 0 | 0 | 0 | 0 | 0 | 0 | 0 |
| | After Oxidation/Activated C | 0 | 0 | 0 | 0 | 0 | 0 | 0 |

The non-catalytic, high-temperature oxidation treatment for reformer condensate produces clean water and H₂O and CO₂ reaction products that are entirely compatible with the reformer system. Much of the required heat can be recouped. In addition, there is ample waste heat to provide the energy required for this step. Some additional electrical heat input would probably be required for the highest temperatures. Although the total power requirements for high-temperature oxidation treatment of reformer condensate have not been fully detailed, it is clear that the power input for high-temperature oxidation of the condensate would be small compared to other reformer power demands (such as electrolysis).

### Example 16

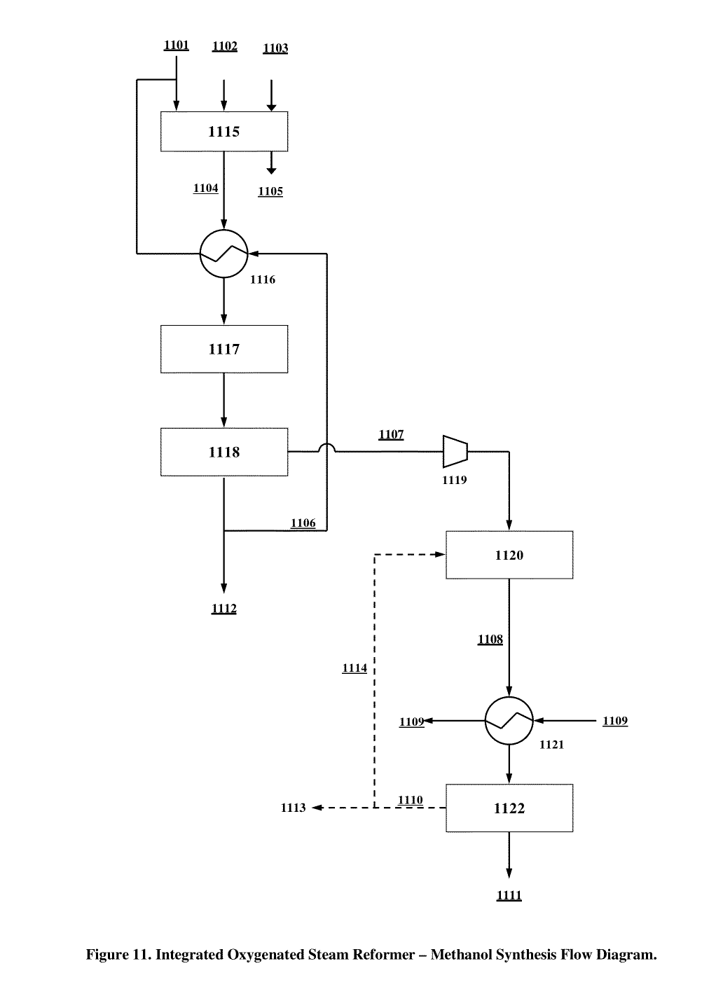

The oxygenated steam reforming procedures described in Examples 1 through 14 are applicable to a process in which the reforming gases are fed to a methanol synthesis reactor system as illustrated in FIG. 11 to produce a significantly greater commercial value from reformer exhaust gases. In this example, the reforming gases are fed to a methanol synthesis reactor after removing moisture.

The dry reforming gases are compressed to a pressure preferably greater than five bar and more preferably to a pressure of at least ten bar and up to 100 bar.

The methanol synthesis reactor is filled with a copper-zinc oxide or other catalyst known for conversion of hydrogen and carbon monoxide to methanol.

The heat release from methanol synthesis is diluted by the presence of unreacted reformer gases including carbon dioxide as well as a portion of the hydrogen and carbon monoxide. A methanol synthesis reactor of the general design described in Example 2 would be suitable for the application. Alternatively, a conventional fixed-bed reactor with appropriate thermal controls is also applicable.

A dry reforming gas produced from agricultural waste material of the composition noted in Example 12 would contain about 30 percent CO₂, 15 percent CO, and 55 percent H₂ (on a molar basis). Feeds to oxygenated steam reforming containing different compositions and produced under different reforming conditions vary from these values. In any case, a significant portion carbon monoxide is converted to the desired methanol product while the large majority of carbon dioxide remains unreacted.

Thermodynamic analyses of the conversion of the dry gases generated from reforming of agricultural waste to methanol are illustrated in the following table over a range of pressures at 230° C. Further improvement to yield can be made at lower operating temperature (constrained by the minimum temperature required to achieve useful reaction rates) and higher operating pressure (constrained by hardware costs).

Percentage Conversion of Carbon from
Dry Reforming Gas to Methanol

| | Yield, % of carbon in feed gas | | | |
|---|---|---|---|---|
| | converted to methanol | | | |
| Yield Parameter | 10 bar | 20 bar | 40 bar | 80 bar |
| CH₃OH Yield from CO + CO₂ | 4.4 | 11.6 | 21.6 | 30.2 |
| CH₃OH Yield from CO | 13.1 | 34.9 | 64.8 | 90.7 |

After removal of the valuable methanol product, remaining gases including carbon dioxide, carbon monoxide, and hydrogen can be further used as fuel or as feedstock to recover additional products.

Optionally, un-reacted carbon monoxide and hydrogen can be recycled to the methanol synthesis reactor to improve the overall yield of methanol.

### Example 17

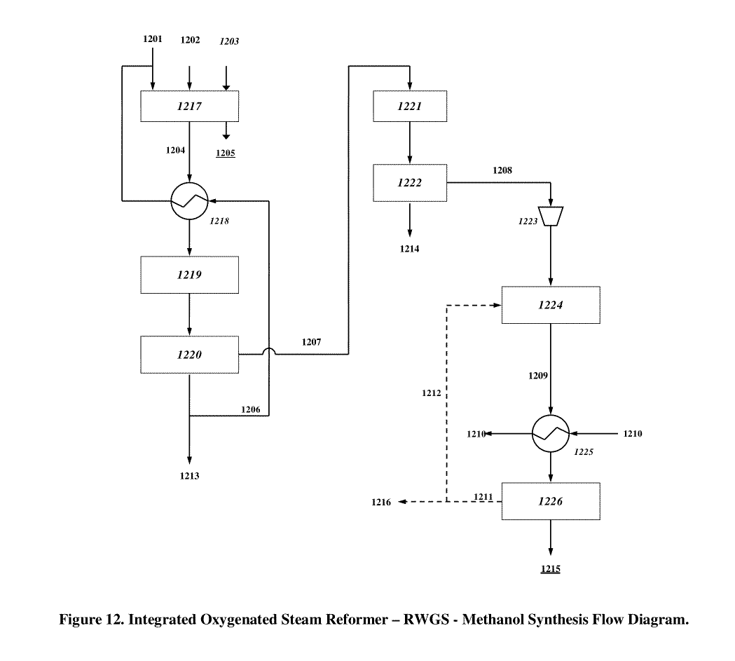

The oxygenated steam reforming procedures described in Examples 1 through 14 are applicable to a process in which the reforming gases are fed to a reverse water gas shift reactor and then to a methanol synthesis reactor system after removal of moisture as illustrated in FIG. 12 to produce a significantly greater commercial value from reformer exhaust gases.

The procedures as described in Example 16 are followed in this example with the exception that a reverse water gas shift (RWGS) reaction is performed on the dry reformer gas before feeding to the methanol synthesis reactor.

The inclusion of the RWGS step (with subsequent removal of water formed) results in a feed gas to methanol synthesis that allows for greater utilization of carbon oxides and hydrogen.

The reverse water gas shift reaction can be carried out over a copper-zinc oxide or other suitable catalyst at a temperature between 300 and 500° C. and pressures of one to 20 bar or more.

The dry reformer gas composition of 30 moles CO₂, 15 moles CO, and 55 moles H₂ is shifted to a composition of approximately 22 moles CO₂, 23 moles CO, and 46 moles H₂. Water is condensed and removed to produce a dry feed gas to methanol synthesis. This feed composition to the methanol synthesis reactor results in improved yield of the desired methanol product. The following table shows the effect of including an RWGS step upstream of the methanol synthesis step (compare to the results shown in the summary table of Example 16).

Percentage Conversion of Carbon from Dry Reforming
Gas to Methanol after First Subjecting the Reformer
Exhaust Gas to a Reverse Water Gas Shift Reaction

| | Yield, % of carbon in feed gas | | | |
|---|---|---|---|---|
| | converted to methanol | | | |
| Yield Parameter | 10 bar | 20 bar | 40 bar | 80 bar |
| CH₃OH Yield from CO + CO₂ | 5.4 | 13.9 | 25.1 | 34.4 |
| CH₃OH Yield from CO | 10.5 | 27.3 | 49.1 | 67.4 |

Results from this example show an improvement in methanol yield from total carbon oxides in the feed gas from 21.6 percent to 25.1 percent (at 230° C. and 40 bar).

After removal of the valuable methanol product, remaining gases including carbon dioxide, carbon monoxide, and hydrogen can be further used as fuel or as feedstock to recover additional products.

Optionally, un-reacted carbon monoxide and hydrogen can be recycled to the methanol synthesis reactor to improve the overall yield of methanol.

### Example 18

In the synthesis of methanol and dimethyl ether described in earlier examples, high pressure is favored to increase the yield of desired products from the feed gases. These synthesis reactions are exothermic and benefit from effective thermal management.

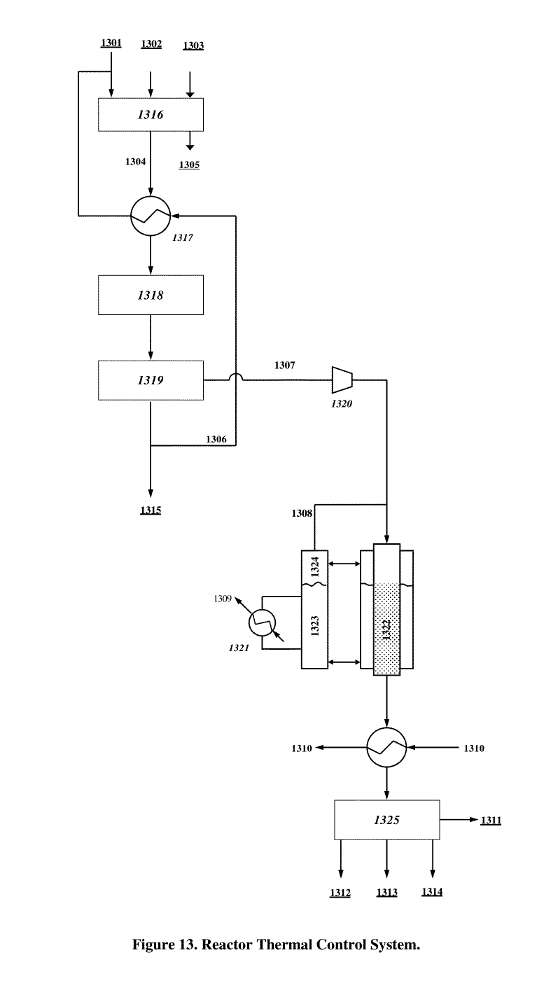

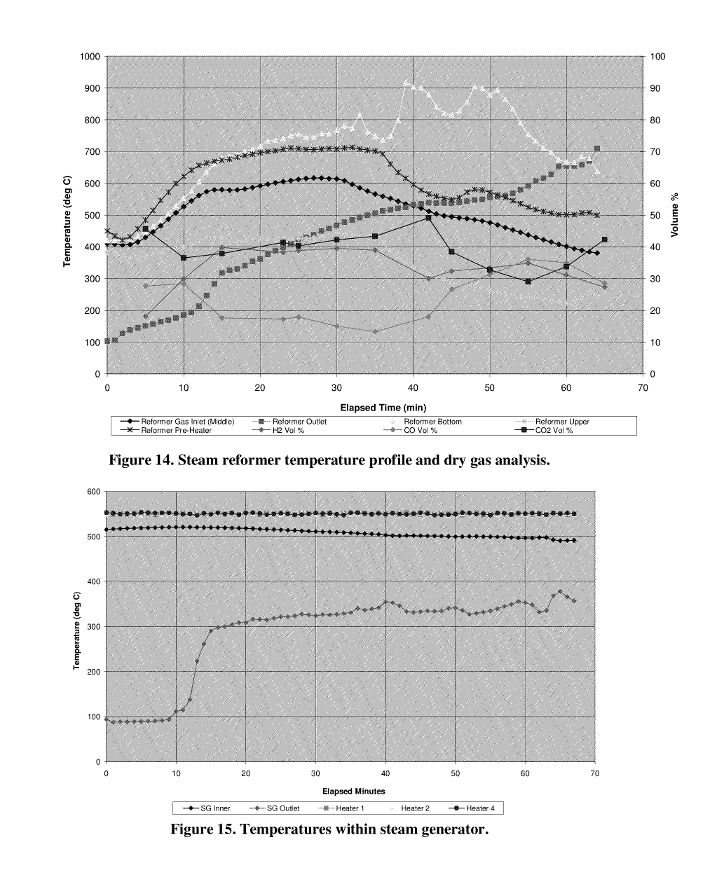

FIG. 13 shows an example of a dimethyl ether synthesis reactor system (similar to that described in Examples 13 and 14) with a novel thermal control system for removal and rejection of the exothermic heat of reaction and for providing a uniform, precise temperature control (also applicable to methanol synthesis or other higher pressure reactions).

The synthesis reactor (typically operating at temperatures between 200 and 250° C.) is surrounded by a jacket containing saturated steam above pressurized water. The surrounding jacket removes heat from the exothermic catalytic synthesis reaction while acting as a heat sink to maintain precise temperature control.

The liquid and head space of the reactor jacket are connected to an adjoining pressure vessel containing pressurized water and saturated steam.

As heat is generated in the synthesis reactor and transferred to the surrounding jacket, the pressurized water and steam flow by convection to and from the adjoining vessel.

A heat exchanger connected to the adjoining vessel is used to remove heat from the pressurized water and saturated steam vessel in order to hold a constant temperature and pressure in the reactor jacket and adjoining vessel, and thereby in the synthesis reactor.

A pressure equalization line connects the head space above the pressurized water vessel to the feed gas from reforming.

The feed gas from reforming (primarily carbon monoxide, carbon dioxide, and hydrogen) is compressed upstream of the pressure equalization line.

In this example, the desired reactor temperature and pressure are the same as that of the pressurized water and saturated steam vessel (about 200 to 250° C., or about 210 to 560 psig).

The pressure equalization line ensures that the reactor pressure is maintained close to that of the pressurized water and steam to minimize risk of collapse of the synthesis reactor shell and to allow for minimum wall thicknesses in the synthesis reactor (by keeping a small differential pressure between the reactor and jacket).

The pressure equalization line is sized so that flow of steam to the inlet gas or vice versa is negligible under most circumstances.

A condensate trap, check valve, or other device may also be installed in the pressure equalization line to further reduce the flow of reactor feed gas to the steam vessel or of steam to the synthesis reactor.

### Example 19

Reformer gas of the type described in Examples 1 through 14 and 16 through 18 can be fed directly to a combustor or internal combustion engine for generation of electricity.

The as-is reformer gas, or preferably, dry reformer gas can be fed to a combustion turbine, conventional gas-fired furnace connected to a steam turbine, or an internal combustion engine connected to a generator. For a typical dry reformer gas containing 55 mole percent hydrogen, 15 mole percent carbon monoxide, and 30 mole percent carbon dioxide, a higher heating value of about 10.7 MJ/kg is available.

Optionally, the dry reformer gas can be separated by membranes, pressure or vacuum swing absorption, liquid absorption, or other methods to remove carbon dioxide. Such a gas containing predominately hydrogen and carbon monoxide would have a higher heating value of about 37.5 MJ/kg. Such a gas can also be fed directly to a system as described above for electricity generation.

Optionally, the dry reformer gas can be separated by membranes, pressure or vacuum swing absorption, liquid absorption, or other methods to remove carbon dioxide and carbon monoxide. Such a gas containing predominately hydrogen would have a higher heating value of about 141.8 MJ/kg. Such a gas can also be fed directly to a system as described above for electricity generation.

A predominately hydrogen containing gas so produced would generate electricity with no carbon emissions when used as fuel for a combustion based power generation system.

### Example 20

Reformer gas of the type described in Example 12 with subsequent methanation can be fed directly to a combustor or internal combustion engine for generation of electricity.

The as-is methanation product gas, or preferably, dry methanation gas can be fed to a combustion turbine, conventional gas-fired furnace connected to a steam turbine, or an internal combustion engine connected to a generator. For a typical dry methanation gas containing about 43 mole percent methane and about 57 mole percent carbon dioxide, a higher heating value of about 11.9 MJ/kg is available.

Optionally, the dry methanation gas can be separated by membranes, pressure or vacuum swing absorption, liquid absorption, or other methods to remove carbon dioxide. Such a gas containing predominately methane would have a higher heating value of about 55.5 MJ/kg. Such a gas can also be fed directly to a system as described above for electricity generation.

### DETAILED DESCRIPTION OF DRAWINGS

FIG. 1 describes a dual reactor system in which an oxygenated (102) steam (101) reformer (113) converts waste feed (103) to reformer-product-gases (H₂, CO₂, CO, H₂O) (104). Prior to entering the methanation reactor (116), the reformer-product-gases are sent through a heat exchanger (114) and then through a sulfur, chloride, fluoride removal system (115). The cooled and cleaned gas stream is then converted to methanation-product-gases (CH₄, CO₂, H₂, H₂O) (106) in the methanation reactor (116). This gas stream is the sent through a heat exchanger (117) and condenser (118) to remove water that is then recycled to the reformer for the steam input. The recycled water (107) goes though both heat exchangers to recover heat from the product gas streams prior to the water entering the reformer. Any excess water (110) is recovered from the condenser. The dry gas stream (CH₄, CO₂, H₂) (109) exits the condenser and is pumped through a compressor (119) and separated in a membrane separator (120) that divides the gas stream into a CO₂-rich product gas (111) and CH₄-rich product gas (114). A portion (108) of the CO₂-rich product gas is recycled to the methanation reactor to promote the conversion of CO and H₂ into methane.

FIG. 2 describes the integrated oxygenated steam reformer-methanation-electrolysis flow diagram. The flow diagram shows the water (201), oxygen (202) and waste feed (203) entering the reformer (221). Any ash or solid contaminants (205) are removed from the reformer after the reforming process. The reformer exhaust stream (204) then enters a condenser (222) in which the reformer condensate (207) exits and a portion (201) is recycled back to the reformer as its steam input and a portion (210) is sent to the electrolyzer (225). The Sabatier reactor (223) has four inputs. The first is the dry reformer exhaust gas stream (206). The other three inputs are generated from the Sabatier exhaust. After the Sabatier exhaust (208) is dried in a condenser (224), the Sabatier condensate (209) enters the electrolyzer (225) along with a portion of the reformer condensate (210). The electrolyzer hydrogen (216) is one of the inputs for the Sabatier reactor. The total electrolyzer oxygen (211) is separated into an oxygen product (213) and a portion (212) is sent to the reformer as its oxygen input. Some make-up water (214) also enters the electrolyzer to complete the electrolysis water input. After exiting the condenser (224), the dry Sabatier exhaust (215) enters a membrane separator (226). The membrane permeate (217) is the third input to the Sabatier reactor. The membrane retentate (218) enters a methane liquefaction system (227) with produces the final methane product (220). The remaining hydrogen (219) is recycled to the Sabatier reactor as its fourth input creating a virtual closed system.

FIG. 3 is a SolidWorks drawing depicting the oxygenated steam reformer vessel, startup boiler and the oxygen mass flow controller used in the experiments.

FIG. 4 shows the schematic of the oxygenated steam reformer vessel used in the experiments.

FIG. 5 describes a one reactor system in which methanation feed gas (501) is converted into water (508) and methane product (505). The methanation feed gas enters into the bottom of the methanation reactor (506) that contains Ruthenium or Nickel catalyst. There is a recycle loop around the methanation reactor with temperature control valves (511) to maintain the temperature from heat released during the exothermic reaction. The recycled gas also goes through a heat exchanger (512) if more control is necessary. The methanation exhaust gas flows through another heat exchanger (514) prior to flowing through a condenser (507) where the gas stream is dried. The water (508) is collected from the condenser. The dry gas (515) flow rate is controlled by a back pressure regulator (516) and monitored with a pressure gauge and pumped (510) at the desired conditions to the membrane separator (509). The membrane permeate (503) is a H₂-rich recycle gas (502) that is combined the methanation feed gas (501) as the inlet gas of the methanation reactor. The membrane retentate (504) flowrate is controlled by a back pressure regulator (520) and exits as the methane product (505). Three sample ports (513, 517, 519) were installed to allow gas chromatograph analysis. FIG. 6 shows a detailed drawing of the methanation reactor that includes two recycle loops to control the temperature of the exothermic reaction.

FIG. 7 describes a two reactor system that involves the integrated oxygenated-steam-reformer dimethyl-ether-synthesis. Water (701), oxygen (702) and organic feed (703) are combined in the steam reformer (706) resulting in the product gases (704) consisting of H₂, CO₂, CO and H₂O. After the reformation process, any ash and solid contaminants (705) are removed. The reformer exhaust gas is cooled by a heat exchanger (707) and cleaned in a sulfur, chloride, and fluoride removal system (708) before entering a condenser (709) where water is removed. The recycled water (710) is preheated by the heat exchanger (704) using the heat from the reformer exhaust gas and is used as part of the reformer water input. The dry gas (CO, CO₂ and H₂) (711) is pumped by a compressor (712) to the methanol-dimethyl ether (DME) synthesis reactor (713). The methanol-DME product gas (716) consists of dimethyl ether, methanol, hydrogen, carbon dioxide, carbon monoxide and water. This gas stream is cooled by a heat exchanger (714) using air, water or other cooling agent (717) and then enters a condenser and separator unit (715) resulting in four exhaust streams of 1) DME (720), 2) methanol (721), 3) water (722) and 4) the remaining H₂,CO₂, and CO (718).

FIG. 8 describes the Lunar Organic Waster Reformer (LOWR) experimental process that was made with Government support under contract NNX11CA76C awarded by NASA. Water from the reservoir (869) is pumped into the steam generator (852), via two heat exchangers (856,857). Residual heat from the Sabatier reactor (854) and the steam reformer (853) outlets are transferred to the water. Once exiting the generator, the steam (876) is mixed with oxygen (877) before entering the steam reformer (853) filled with biomass. After the reformer, the product gas (878) goes through the first condenser (858) to remove water from the line, which is then recycled (889) back to the water reservoir after first going through the water cleaning system (867, 868). This water flows back into the boiler for steam generation or flows to the electrolyzer (871) via a water pump (870) to decompose into oxygen (877) and hydrogen (881). Some of this oxygen (877) feeds into the steam inlet and the remaining oxygen is accumulated (872) as product gas. The generated hydrogen gas (881) flows into the Sabatier reactor (854) to react with the reformer output (883) producing predominantly methane and water (884). The Sabatier product stream flows through the condensing system (859) to remove the water (889). The water is sent though the cleaning system (867, 868) and either sent to the water reservoir (869) for steam generator (852) input or to the electrolyzer (871) for separation into hydrogen (881) and oxygen (877). The dried Sabatier product (886) recycles through a membrane separator (855). Methane (888) is a product and accumulates in a storage tank while hydrogen (882) is recycled to the Sabatier reactor (854) as needed. Thermocouples (801-821), pressure transducers (822-839) monitor the system conditions. Relief valves (840-841) promote safety. Back pressure regulators (847-849) optimize flowrates. A forward pressure regulator (850) and pressure drop through narrow tubing controls the oxygen flowrate. Gas chromatograph ports (842-846) allow the analysis of the gas stream in a variety of locations through the process. An oxygen sensor (890) is a safety to ensure all oxygen is converted in the steam reformer. A methane/carbon dioxide sensor (875) allows fine tuning and monitoring of the process.

FIG. 9 is a SolidWorks drawing of the condensing system which is comprised of an inner chamber surrounded by a coolant jacket. Product gases from the reformer pass through the condenser in order to remove water and recycle it. To control condenser temperatures, a chilling unit was chosen to circulate an ethylene glycol solution through the system. Gas enters the chamber through a quarter inch tube from the top of the vessel. The quarter inch tube is fitted into a tee, of which the other two ports are sized for ⅜^{th} inch tube. This allows space for the gas to exit the quarter inch tube and then make its way back up and out of the tee fitting. Upon reaching the cold zone inside the condenser, the steam is condensed to water and removed from the gas. The remaining gas then exits the chamber out of the top of the condenser. Water is periodically released from the inner chamber by a ball valve in the exit line at the base of the condenser.

FIG. 10 describes a three reactor system in which organic feed (1003) is first reformed in a steam reformer (1017) using water (1001) and oxygen (1002) and then the exhaust gas flows through a RWGS reactor prior to the methanol-DME synthesis reactor. The reformer product gases (1004) are cooled through a heat exchanger (1018) and cleaned with a sulfur, chloride, fluoride removal system (1019). Water is then condensed (1020) and collected (1016). The dry gas (1007) is then sent to a reverse water gas shift reactor (RWGS) (1021). The inclusion of the RWGS step (with subsequent removal of water (1009) formed with a condenser (1022)) results in a dry feed gas (1008) to methanol-dimethyl ether (DME) synthesis (1024) that allows for greater utilization of carbon oxides and hydrogen. The methanol-DME synthesis reactor product gases (1010) are cooled in a heat exchanger (1025) that utilizes air, water or another cooling agent (1011) and is then sent through to a condenser/separator unit (1026) producing four product streams consisting of dimethyl ether (1013), methanol (1014), water (1015) and the remaining H₂, CO₂, and CO (1012).

FIG. 11 describes a two reactor system in which the oxygenated steam reformer product gases are sent through a methanol synthesis reactor to produce methanol (1111). Organic feed (1103) is reformed in a steam reformer (1115) using water (1101) and oxygen (1102). The product stream is cooled in a heat exchanger (1111) and cleaned in a sulfur, chloride, fluoride removal system (1117). It is then dried in a condenser (1118) in which part of the water is collected (1112) and part is recycled (1106) to use in the steam reformer (1115). The recycled water is preheated through a heat exchanger (1116) that transfers the heat from the steam reformer product gases (1104). The dry gas (1107) is pumped by a compressor (1119) to a methanol synthesis reactor (1120) The exhaust gas is cooled in a heat exchanger (1121) using air, water or another cooling agent (1109) and then sent to a condenser/separator unit (1122) but in this case the final product is methanol (1111) and there an optional recycle (1114) to the methanol synthesis reactor (1120) of the H₂, CO₂ and CO (1110). There is a vent (1113) if the optional recycle is not used.

FIG. 12 describes a three reactor system in which the oxygenated steam reformer product gases are sent first through a reverse water gas shift reactor (RWGS) (1221) and then through a methanol synthesis reactor to produce methanol (1215). Organic feed (1203) is reformed in a steam reformer (1217) using water (1201) and oxygen (1202). The product stream is cooled in a heat exchanger (1218) and cleaned in a sulfur, chloride, fluoride removal system (1219). It is then dried in a condenser (1220) in which part of the water is collected (1213) and part is recycled (1206) to use in the steam reformer (1217). The recycled water is preheated through a heat exchanger (1218) that transfers the heat from the steam reformer product gases (1204). In this scenario, the dry gas (1207) flows through the RWGS reactor (1221) and the exhaust has is dried in a condenser (1222). This dry gas (1208) is pumped by a compressor (1223) to a methanol synthesis reactor (1224) The exhaust gas is cooled in a heat exchanger (1225) using air, water or another cooling agent (1210) and then sent to a condenser/separator unit (1226) in which the final product is methanol (1215) and there an optional recycle (1212) to the methanol synthesis reactor (1224) of the H₂, CO₂ and CO (1211). There is a vent (1216) if the optional recycle is not used.

FIG. 13 details the novel thermal control system for removal and rejection of the exothermic heat of reaction and for providing a uniform, precise temperature control used for the methanol-DME synthesis reactor (1322). The synthesis reactor (1322) is surrounded by a jacket containing saturated steam above pressurized water. The surrounding jacket removes heat from the exothermic catalytic synthesis reaction while acting as a heat sink to maintain precise temperature control. The liquid and head space of the reactor jacket are connected to an adjoining pressure vessel containing pressurized water (1323) and saturated steam (1324). As heat is generated in the synthesis reactor and transferred to the surrounding jacket, the pressurized water and steam flow by convection to and from the adjoining vessel. A heat exchanger (1321) connected to the adjoining vessel is used to remove heat from the pressurized water and saturated steam vessel in order to hold a constant temperature and pressure in the reactor jacket and adjoining vessel, and thereby in the synthesis reactor. A pressure equalization line (1308) connects the head space above the pressurized water vessel to the feed gas from reforming. The dry feed gas (1307) from reforming (primarily carbon monoxide, carbon dioxide, and hydrogen) is compressed (1320) upstream of the pressure equalization line. The dry reformer gases (1307) are made from organic feed (1303) reformed in a steam reformer (1316) using water (1301) and oxygen (1302). The product stream is cooled in a heat exchanger (1317) and cleaned in a sulfur, chloride, fluoride removal system (1318). It is then dried in a condenser (1319) in which part of the water is collected (1315) and part is recycled (1306) to use in the steam reformer (1316). The recycled water is preheated through a heat exchanger (1317) that transfers the heat from the steam reformer product gases (1304). After the dry gas is goes through the methanol-DME synthesis reactor (1322), it is cooled in a heat exchanger (1310) and condensed and separated (1325) resulting in dimethyl ether (1312), methanol (1313), water (1314) and H₂, CO₂ and CO.

What is claimed is:

1. A process for oxygenated steam reforming of organic matter to a gas product of hydrogen, carbon dioxide, carbon monoxide, and water comprising:
a. Injection of oxygen and steam preheated by process waste into a reaction zone along with said organic matter;
b. Operating the process at temperatures above 400 C and up to 1,500 C in the reformer section;
c. Operating the process at pressures near atmospheric pressure to 200 bar in the absence of a catalyst; and,
d. Partially combusting said organic matter with oxygen to generate heat that is used to reform the remainder of the organic matter in the presence of steam to form said product gas.

2. The method of claim 1 in which the organic matter is gas, liquid, or solid household wastes, organic agriculture, forestry, fishing wastes, organic construction wastes, organic manufacturing or industrial wastes, organic municipal or sanitary wastes, organic medical wastes, organic chemicals, fuels, organic matter from chemicals, fuels including those derived from petroleum, lignite, coal, shale, natural gas, or mixed organic wastes including those produced at human space exploration outposts in which the organic matter fed to the reformer consists of as-is material, a shredded, chopped, crushed, or ground feed, or feed otherwise reduced in size or compacted or pelletized to achieve the particle size range optimum for reforming.

3. The method of claim 1 in which water for steam reforming is fed at rates sufficient for reforming, in excess of that for reforming and introduced manually, by automatic controls, and is premixed with organic matter, pumped, injected separately, mixed with an oxygen source selected from atmospheric air, cryogenically produced pure oxygen source, concentrated or partially concentrated oxygen generated by pressure-swing, vacuum-swing, temperature-swing, absorption methods, membrane separation, or other oxygen concentration or nitrogen removal devices.

4. The method of claim 1 in which chemical compounds or absorbents are added to the organic matter to trap contaminants such as sulfur, chlorine, fluorine, and other contaminants contained in the feed and released during reforming or are located in the hot, moist reformer exhaust, or are located in the dried reformer exhaust.

5. The method of claim 1 in which charcoal, carbon, or unprocessed organic matter is loaded into the reformer at a location downstream of the reaction zone to provide a physical support for the organic matter being processed and to provide a back up source of fuel in the event of feed depletion or other upset.

6. A process for production of high quality methane in a methanation reactor system by reacting carbon monoxide and hydrogen in a methanation reactor system comprising:
a. Efficient thermal management via internal and external heat exchangers
b. Product gas separation via membranes; and,
c. Gas recycling via gas compressors.

7. The method of claim 6 in which the methanation reactor feed gases are first passed through an indirect heat exchanger contained within the methanation reactor

8. The method of claim 6 in which the product gas resulting from methanation is used directly as fuel or other uses or is stored for later use.

9. The method of claim 6 in which the ratio of hydrogen to carbon oxides is varied by separating component gases to enable complete carbon oxide conversions to methane at higher hydrogen ratios.

10. The method of claim 6 in which a part or all of the water present in the steam reformer and/or the methanation reactor exhaust is condensed and fed to an electrolysis system for production of hydrogen and oxygen.

11. A device for oxygenated steam reforming of organic matter to produce a gas containing predominately hydrogen, carbon dioxide, carbon monoxide, and water where product gas may be used as feed for electricity generation, methanation reactions, methanol and dimethyl ether synthesis, and other organic chemical conversion reactions comprising:
a. A feeding system to deliver organic matter to a reaction zone containing an injector arrangement for oxygen and steam preheated by waste heat for controlled addition of oxygen and steam in the absence of catalyst,
b. A grid to support reacted residue and to allow reformer gases to pass to the exhaust gas line,
c. A residue removal system,
d. A heat exchanger to transfer waste heat from reformer exhaust gas to the oxygen and steam feeds,
e. A trap to capture contaminants contained in the organic matter feed, and
f. A condenser system to remove water from reformer gases.

12. The device of claim 11 which is equipped with a feed magazine of a size required to provide continuous feed of organic matter for a designated operating time, a semi-continuous feed system such as a lock-hopper, auger, or other feeding device to allow for extended operating time and in which the organic matter is metered manually or via automatic controls.

13. The device of claim 11 in which oxygen and steam are metered manually, metered through an automated feed control system, fed separately into the reformer, metered through an automated feed control system and are passed through a mixer prior to injection into the reformer and in which a concentric jacket surrounding all or a portion of the reformer length is installed for purposes of cooling the reformer reactor shell preheating oxygen and steam and in which single or multiple ports are installed to allow the oxygenated steam to enter the reformer reaction zone at one or more positions along the reformer length.

14. The device of claim 11 in which a plate or disk or screen is installed at a location downstream of the reaction zone to provide a physical support for charcoal, carbon, or organic matter residue including a mechanism such as an auger, lock hopper, or other material flow device installed for continuously or periodically removing accumulated inorganic matter, unreacted organic matter, a mechanism to allow the oxygenated steam reforming vessel to be inverted for periodic removal of accumulated inorganic matter and/or unreacted organic matter through the feed flange or seal system, a pneumatic or vacuum system for removal of accumulated inorganic matter and unreacted organic matter through the feed flange or seal system when the system is idle.

15. The device of claim 11 in which a boiler or steam generator is installed to vaporize water during start up of the reformer and to further increase the temperature of water that is preheated and/or vaporized via heat exchange from the reformer system, methanation system, or other downstream reaction system.

16. The device of claim 11 in which a heat exchanger is installed for cooling or partially cooling the hot steam reforming exhaust gases using water, steam, or oxygen fed to the reformer or using separate air, water, or other cooling and chilling media and in which a condenser is used to remove a part of or all of the moisture prior to feeding gases to downstream methanation or other reaction systems.

17. A device to perform methanation of carbon monoxide and carbon dioxide gases with hydrogen comprising:
a. an integrated internal indirect heat exchanger
b. a recycle gas compressor, and
c. a gas separation membrane for recycle of unreacted hydrogen and carbon oxides to the catalytic reactor zone

18. The device of claim 17 in which provision is made to allow moisture present in the reforming gas to remain in the methanation reactor feed to prevent carbon formation.

19. The device of claim 17 in which provision is made to load catalyst comprising ruthenium, nickel, or other suitable catalyst into the methanation reactor.

20. The device of claim 17 in which internal and external heaters are installed for start up and temperature control between 250 C and 600 C in which materials of construction are selected to allow for operation at pressures near ambient to 200 bar.

21. The device of claim 17 in which provision is made to inject supplemental hydrogen produced by water electrolysis or other means to allow for methanation reactions to take place under substoichiometric hydrogen conditions, stoichiometric conditions, or excess hydrogen conditions for which hydrogen can be used to control reaction extent and reactor temperatures.

22. The device of claim 17 in which an indirect heat exchanger is installed inside the methanation reactor to transfer reaction heat to the methanation reactor feed gases which are first passed through said heat exchanger and create within a single vessel first a favorable reaction zone in which methanation reactions are carried out at relatively high rates at relatively higher temperature and second a favorable reaction zone in which methanation reactions are carried out to a relatively high equilibrium conversion at relatively lower temperature and in which provisions are installed to subsequently cool or partially cool said gas via radiative, convective, or conductive heat exchange prior to introduction to the methanation reactor catalyst zone.

23. The device of claim 17 in which a heat exchanger is installed to indirectly cool or partially cool the hot methanation reactor exhaust gases against water, steam, or oxygen being fed to the reformer, indirect radiative heat exchanger using air or other cooling media including a chiller to cool or partially cool methanation reactor exhaust gases and to condense and remove moisture contained in the methanation reactor exhaust gases.

24. The device of claim 17 in which an exhaust system is installed to direct the dry product gas resulting from methanation for use as fuel, other purposes, or storage installed for later use of methanation product gas.

25. The device of claim 17 in which a gas compressor and separation membrane are installed for recovery of carbon dioxide-rich, hydrogen plus carbon dioxide-rich, or hydrogen-rich gases that are partially or entirely recycled to the methanation reactor simultaneous with recovery of methane-rich gas that is used directly as fuel, stored, sold, or used as a feed to downstream reaction systems.

26. The device of claim 17 in which an electrolysis system is installed to process part or all of the condensed water present in the steam reformer exhaust, methanation reactor exhaust for production of hydrogen and oxygen gas with provision to direct hydrogen to the methanation reactor and oxygen to the oxygenated steam reforming reactor with provisions to compress, liquefy, store, sell hydrogen and oxygen produced in excess of reforming and methanation process requirements.

27. The device of claim 17 in which an infrared gas analyzer or other analysis hardware is installed to determine the composition of the product gas from methanation in near real time in which a data acquisition and control system is installed using said composition data for feedback control to adjust the hydrogen addition rate to the methanation reactor and to adjust the electrolyzer rate to produce hydrogen accordingly.

28. A device to remove organic matter such as tar and dissolved organic matter from steam reforming condensate comprising:
a. Oxidation in aqueous phase, or
b. Oxidation in gaseous phase.

29. The device of claim 28 in which provision is made to inject oxygen, hydrogen peroxide, other oxidizing materials into a non-catalytic reaction zone to oxidize tars and organic matter present in condensate in aqueous or gaseous form at temperatures above ambient to 1000 C and pressures near ambient to 200 bar, into a catalytic reaction zone containing copper or other suitable wet oxidation catalysts to oxidize tars and organic matter present in condensate in aqueous or gaseous form at temperatures above ambient to 1000 C and pressures near ambient to 200 bar.
